(19) United States
(12) Patent Application Publication
(10) Pub. No.: US 2025/0215455 A1
(43) Pub. Date: Jul. 3, 2025

(54) GENETIC TARGETING OF CELLULAR OR NEURONAL SUB-POPULATIONS

(71) Applicant: BOARD OF REGENTS, THE UNIVERSITY OF TEXAS SYSTEM, Austin, TX (US)

(72) Inventor: Boris V. ZEMELMAN, Austin, TX (US)

(73) Assignee: BOARD OF REGENTS, THE UNIVERSITY OF TEXAS SYSTEM, Austin, TX (US)

(21) Appl. No.: 19/049,184

(22) Filed: Feb. 10, 2025

Related U.S. Application Data
(62) Division of application No. 17/431,847, filed on Aug. 18, 2021.
(60) Provisional application No. 62/807,366, filed on Feb. 19, 2019.

(51) Int. Cl. C12N 15/86; C12N 15/63

(52) U.S. Cl. CPC C12N 15/86; C12N 15/635; C12N 2750/14143; C12N 2830/001; C12N 2830/008; C12N 2830/48; C12N 2830/50

(57) ABSTRACT

In some aspects, promoters, vectors, and methods of selectively inducing expression in subtypes of neuronal cells are provided.

This application is a divisional of U.S. application Ser. No. 17/431,847, filed Aug. 18, 2021, as a national phase application under 35 U.S.C. § 371 of International Application No. PCT/US2020/018759, filed Feb. 19, 2020, which claims the priority benefit of U.S. provisional Application No. 62/807,366, filed Feb. 19, 2019, the entirety of each of which are hereby incorporated by reference.

This invention was made with government support under Grant No. U01 NS094330 and U01 NS094362 awarded by the National Institutes of Health. The government has certain rights in the invention.

### BACKGROUND OF THE INVENTION

This application contains a Sequence Listing XML, which has been submitted electronically and is hereby incorporated by reference in its entirety. Said XML Sequence Listing, created on Feb. 7, 2025, is named UTSBP1207USD1.xml and is 38,575 bytes in size.

### 1. Field of the Invention

The present invention relates generally to the field of molecular biology and regulation of mammalian gene expression. More particularly, it concerns genetic methods and constructs for expressing heterologous proteins in neuronal populations.

### 2. Description of Related Art

Viral vectors enable transgenics-independent protein expression in the primate brain. However, viruses targeting specific neuron classes have proven elusive. More specifically, functional dissection of mammalian neuronal circuits is predicated on an ability to accurately target constituent cell classes. Transgenic approaches in rodents, particularly in mice, have proven useful, offering a precise and predictable way to access genetically-defined cell populations for subsequent manipulations (He et al., 2016; Murray et al., 2012; Taniguchi et al., 2011). However, rodent line derivation represents a trade-off between reliability and convenience: costly and time-consuming techniques designed to produce genetic animal models are poor vehicles for expressing engineered proteins that can become obsolete during the animal's lifespan. There is also the pressing need to target genetically and molecularly specified neuronal populations in the primate, an important animal model for human perception, cognition, and action, which is less amenable to genomic manipulations. Viral vectors represent an attractive alternative to transgenic rodents and have been used to express heterologous proteins (Betley and Sternson, 2011). These vectors, such as recombinant adeno-associated viruses (rAAVs), are non-pathogenic, infect neurons of multiple species, and offer the added benefits of spatial and temporal control over transgene expression (Samulski et al., 1989; Tenenbaum et al., 2004).

One shortcoming of viral vectors, however, has been their limited cell type-specificity in the brain: with the few exceptions of pan-neuronal and excitatory neuron targeting (Borghuis et al., 2011; Dittgen et al., 2004; Han et al., 2009; Kügler et al., 2003; Schoch et al., 1996; Seidemann et al., 2016), restricting heterologous protein expression to subsets of excitatory and inhibitory neurons using viruses has proven difficult (Nathanson et al., 2009b; Dimidschstein et al., 2016; Lee et al., 2014). This is because the mechanisms of cell type-specific gene expression regulation are not well understood: it is currently impossible to predict whether and how a particular DNA domain or region will affect nearby gene expression. Promoter elements have been identified for some specialized cell classes through direct trail-and-error testing in the brain, but not for the cell classes described here. Moreover, because the size of the viral genome is limited, it is not possible to use very large chromosomal segments that may encompass regulatory domains, which is a workaround prevalent in mouse transgenics.

It has historically been difficult to restrict virus-encoded protein expression to subsets of cells. In the brain, where numerous cell types are known to reside, the challenge is especially profound. Brain cells comprise neurons and glial cells. There are three major classes of neurons: excitatory, inhibitory and modulatory. Each class is composed of multiple subclasses with distinct functions, morphology and anatomical connections. In addition, the mammalian cortex is a layered structure-neurons that are members of a single subclass carry out different functions in different cortical layers. Combinations of neurons form neuronal circuits and networks that process sensory and physiological information, retain and recall memories, and generate behaviors. Accessing neuronal subclasses is essential for understanding and influencing brain circuitry that governs perception and action.

Functionally relevant subclasses of excitatory and inhibitory neurons typically do not fall within clear boundaries with respect to intrinsic neurochemical markers (Soltesz and Losonczy, 2018), and most neuronal genes are expressed at different levels in many neuron subclasses (Tasic, 2016; Cembrowski and Menon, 2018; Lein et al., 2007), making it very difficult to define subclasses based on single unique genetic markers. Clearly, there is a need for new methods for selectively targeting protein expression to neuronal subclasses, such as for example GABAergic interneuron subclasses, and these methods have to reflect and harness the complexity of gene expression patterns in the brain.

It has not been possible to target all GABAergic (inhibitory) neurons using viruses. Prior efforts used viruses to target many, but not all, inhibitory neurons (Dimidschstein et al., 2016; Lee et al., 2014). It has not been possible to target specific subclasses of inhibitory neurons with respect to brain region, cortical layer, function, or genetic markers using viruses.

The targeting of all excitatory neurons with viruses is generally achieved using a section of the mouse calcium/calmodulin-dependent protein kinase II alpha (CaMKIIa) promoter (Dittgen et al., 2004). However, under certain conditions this promoter may also be active in inhibitory interneurons (Nathanson et al., 2009a; Schoenenberger et al., 2016) and inactive in subsets of cortical excitatory neurons (Huang et al., 2014; Wang et al., 2013; Watakabe et al., 2015). Moreover, there is considerable regional variation in the expression of endogenous CaMKIIa in mammalian cortex as well as in extracortical brain structures (Benson et al., 1992; 1991). Subclasses of excitatory neurons, with respect to brain region, cortical layer, function, or genetic markers cannot currently be targeted using viruses with specific promoters.

Accessing neuronal subclasses is essential for unraveling brain circuitry that governs animal perception and behavior. However, functional studies have revealed that the relevant cell ensembles-excitatory or inhibitory-rarely fall within neat neurochemical boundaries (Soltesz and Losonczy, 2018). Moreover, neuronal gene expression is both promiscuous and variable (Tasic, 2016; Cembrowski and Menon, 2018; Lein et al., 2007), making it difficult to find single surrogate markers for the emerging functional classes. Clearly, there is a need for new methods for selectively targeting expression in neuronal subtypes, such as for example GABAergic interneurons, as well as a need for improved methods that can reflect and harness the complexity of gene expression patterns in the brain.

### SUMMARY OF THE INVENTION

The present invention overcomes limitations in the prior art by providing compositions and methods for genetically accessing neuronal sub-populations. In some embodiments, single promoters can be used to restrict access to sub-populations of neurons. In some embodiments, single promoters active in different sub-populations of neurons can be used together to access a larger sub-population of neurons than either promoter alone (“set summation”). In some embodiments, use of single promoters that have overlapping, but distinct, patterns of expression in different neuronal populations may be used together to turn on (“set intersection”) or turn off (“set difference”) expression of a functioning expressible gene (e.g., a reporter gene or a therapeutic gene) in cells where both promoters are active. The promoters can be from the same species or a different species from the cell. The promoters can be from DNA regions proximate to genes that are normally active in the accessed sub-populations of neurons. The promoters can be from DNA regions proximate to genes that are not normally active in the accessed sub-populations of neurons but have attained the ability to regulate gene expression in said sub-populations of neurons through change in orientation, a change in sequence, or by being used in neurons of a different species. The promoters can additionally be truncated regulatory regions that support transgene expression in different cell types depending on the brain region where they are introduced (e.g., using viral delivery); for example, a promoter may be active in one class of neurons in the mammalian forebrain, but a different class of neurons in the mammalian brainstem. These approaches may also be used, in some embodiments, to enable the targeting of neuron populations that aren't currently accessible using existing transgenic animals, in parallel with and independently of neuron sub-populations accessed using existing transgenic animals, or by further restricting the neuron sub-population accessed in existing transgenic animals.

For example, two or more promoters can be used intersectionally. Expression of a recombinase or transposase by a second promoter (e.g., via a hybrid promoter in neuronal cells) may be used to cause a deletion or inversion of a separate expressible gene driven by a first promoter, wherein the deletion or inversion results in changing the functionality of the separate expressible gene (e.g., from non-functional to functional) in a cell such as, e.g., a neuron. In this way, only cells (e.g., neurons) that express both the first promoter (“F”) and the second promoter (“S”) will express of the functionally-altered (i.e., functional) separate expressible gene (“F and S; set intersection”). Alternatively, expression of a recombinase (e.g., Cre/Flp/Dre) or transposase by a second promoter (e.g., via a hybrid promoter in neuronal cells) may be used to cause a deletion or inversion of a separate expressible gene driven by a first promoter, wherein the deletion or inversion results in changing the functionality of the separate expressible gene (e.g., from functional to non-functional) in a cell such as, e.g., a neuron. In this way, cells (e.g., neurons) that express only the first promoter (“F”) but not the second promoter (“S”) will express the functionally-altered (i.e., functional or non-functional) separate expressible gene (“F not S; set difference”). In another example, expression of the repressor by a second promoter (e.g., via a hybrid promoter in neuronal cells) may silence or repress expression of the expressible gene by the first promoter. In this way, cells (e.g., neurons) that express only the first promoter (“F”) but not the second promoter (“S”) will express the functionally-altered (i.e., functional or non-functional) separate expressible gene (“F not S; set difference”). Thus, previously genetically inaccessible neuronal sub-populations may be genetically accessed by using a first and second promoter to drive expression in different, but overlapping, populations of neurons. Expression of a recombinase can thus be used to turn expression of a gene or transgene on or off, or a repressor can thus be used to turn off expression of a gene or transgene. In some aspects, synthetic enhancer regions such as h56D are provided and may, e.g., be included with a minimal promoter to form a hybrid promoter, and in some embodiments the synthetic enhancer may be used to drive expression in neuronal cells. In particular embodiments, and as shown in the below examples, methods and compositions provided herein may be particularly useful for causing genetic expression in neuronal sub-populations in the primate brain or human brain. This targeting can be combined with transgenics (e.g., a transgenic mouse) to drive expression in a targeted sub-population of neurons that is more specific and/or refined beyond the expression by the transgene alone. The promoters may be from the same species as the cell or from a different species. In some preferred embodiments, promoters are used from a gene that is expressed in a different cell type from cell that is being targeted for altered expression of one or more transgenes; for example, in some embodiments, a domain or promoter near calbindin is used to drive expression in cholecystokinin cells (CCK cells), and/or a domain or promoter from PaqR4 is used to target parvalbumin (PV) inhibitory cells. In some embodiments, the expression of a gene (e.g., a reporter gene or a therapeutic gene) may be selectively induced or repressed in populations of GABAergic interneurons, excitatory neurons, or neuropeptide-Y positive interneurons. In some embodiments more than two promoters may be used, combining repressor and recombinase systems. For example, a h12R promoter may repress expression from h56D promoter to yield neuropeptide-Y positive interneurons. In some embodiments, a recombinase expressed from the somatostatin (SST) promoter (or the PaqR4 promoter for PV cells) may activate or inactivate transgene expression (depending on whether the transgene is non-functional or functional, respectively, at the outset) in SST-positive or PV-positive cells to enable transgene expression only in NPY-positive cells that are also SST or PV-positive (set intersection) or only NPY-positive cells that are additionally SST or PV negative (set difference). In some embodiments, a recombinase expressed from the Rnf promoter may activate or inactivate transgene expression in layer 4 of mammalian cortex. In some embodiments, a recombinase expressed from the Rnf promoter may activate or inactivate transgene expression from the SST or Paqr4 promoters in layer 4 of mammalian cortex, limiting the change in transgene expression to layer 4 SST or PV neurons. In some embodiments, a recombinase expressed from the h56R promoter fused to CMV enhancer may activate or inactivate transgene expression in layer 4 of mammalian cortex.

An aspect of the present invention relates to a method of inducing expression in a cell comprising contacting the cell with one or more nucleic acids encoding: (i) a first promoter operably linked to a first expressible gene, and (ii) a second promoter operably linked to a first recombinase, a transposase, or a repressor; wherein the first promoter and the second promoter each induce expression in overlapping, but different, populations of neurons; wherein expression of the recombinase or transposase by the second neuronal promoter can result in deletion or inversion of the first expressible gene, and wherein expression of the repressor can silence or prevent the expression of the first expressible gene; and wherein the cell is preferably a neuronal cell. The first promoter and/or the second promoter may be from a species that is different from the cell. The first promoter may be a hybrid promoter comprising an enhancer and a minimal promoter. The first enhancer may comprise or consist of h56D, h56R, h12R, h12D, mSST, hPaqR4, hPaqR4.P3, Rnf208.1, Unc5d.1, CB3, CMV enhancer with NRSE, or h12A. The minimal promoter may be a minimal CMV promoter, a minimal Na/K ATPase promoter, or a minimal Arc promoter. The second promoter may be a hybrid promoter comprising a enhancer and a minimal promoter. The enhancer may comprise or consist of h56D, h56R, h12R, h12D, h12A, mSST, hPaqR4, hPaqR4.P3, Rnf208.1, Unc5d.1, CB3, CMV enhancer with NRSE, or h12A. The minimal promoter may be a minimal CMV promoter, a minimal Na/K ATPase promoter, or a minimal Arc promoter. The first promoter and/or the second promoter may be a neuron-specific, cortical layer-specific, or neuronal promoter. In some embodiments, individual cell-specific promoters may be truncated or extended to achieve a new pattern of transgene expression. In some embodiments, the neuronal promoter is a pan-neuronal human synapsin promoter (hSYN), pan-neuronal mouse synapsin promoter (SYN), parvalbumin (PV) promoter, somatostatin (SST) promoter, neuropeptide-Y (NPY) promoter, vasoactive intestinal peptide (VIP) promoter, CamKIIalpha, CCK (CB3), calbindin, or PaqR4. The first promoter and/or the second promoter may comprise a neuron-specific silencing element or a cortical layer-specific silencing element. In some embodiments, individual cell-specific promoters and enhancers may be combined (fused together) to achieve cell-specific and layer-specific transgene expression. In some embodiments, a pan-neuronal promoter and an enhancer may be combined to achieve expression in all neurons within a single cortical layer. In some embodiments, the expressible gene encodes an inhibitory nucleic acid sequence. The inhibitory nucleic acid sequence may be a small interfering RNA (siRNA), a short hairpin RNA (shRNA) or micro RNA (miRNA). The expressible gene may encode a reporter polypeptide, an ion channel polypeptide, a cytotoxic polypeptide, an enzyme, a cell reprogramming factor, a drug resistance marker, a drug sensitivity marker or a therapeutic polypeptide. In some embodiments, the reporter polypeptide is a fluorescent or luminescent polypeptide. In some embodiments, the expressible gene encodes GCaMP6f. In some embodiments, the fluorescent or luminescent polypeptide is GFP, EGFP, or tdTomato. In some embodiments, the cytotoxic polypeptide is gelonin, a granzyme, a caspase, Bax, Apo-1, AIF, TNF-alpha, a bacterial clostridium neurotoxin catalytic subunit, or a diphtheria toxin catalytic subunit. In some embodiments, the reporter polypeptide comprises a destabilizing domain. In some embodiments, the recombinase is a Cre, Flp, or Dre recombinase. The recombinase may comprise a destabilizing domain. The recombinase may comprise an ER and/or PR domain. The recombinase may comprise at least two destabilizing domains. In some embodiments, expression of the recombinase causes an inversion of or in the first expressible gene. In some embodiments, the inversion results in a functional version of the first expressible gene. In some embodiments, the inversion results in a non-functional version of the first expressible gene.

In some embodiments, the second promoter results in expression of a first recombinase, and wherein the first recombinase is at least partially inverted or contains an inactivation region; wherein the method further comprises contacting the neuronal cell with a third promoter operably linked to a second recombinase; and wherein expression of the second recombinase can result in an inversion or deletion in the recombinase that activates enzymatic activity in the first recombinase. In some embodiments, the third promoter is a hybrid promoter comprising an enhancer and a minimal promoter. In some embodiments, the first enhancer comprises or consists of h56D, h56R, h12R, h12D, mSST, hPaqR4, hPaqR4.P3, Rnf208.1, Unc5d.1, CB3, CMV enhancer with NRSE, or h12A. In some embodiments, the minimal promoter is a minimal CMV promoter, a minimal Na/K ATPase promoter, or a minimal Arc promoter. In some embodiments, the third promoter is a neuron-specific or neuronal promoter. The neuronal promoter may be, e.g., PaqR4 promoter, a pan-neuronal human synapsin promoter (hSYN), somatostatin (SST) promoter, vasoactive intestinal peptide (VIP) promoter, CamKIIalpha, or calbindin. In some embodiments, the first recombinase and the second recombinase are each independently a Cre, Flp, or Dre recombinase. In some embodiments, the second promoter is operably linked to an operator, and wherein the repressor is TetR, MphR, VanR, TtgR or a ligand binding polypeptide fused to a kox-1 protein domain. The one or more nucleic acids may be comprised in a plasmid expression vector or an episomal expression vector. The vector may be a viral expression vector such as, e.g., an adenovirus, adeno-associated virus, a retrograde virus, retrovirus, herpesvirus, lentivirus, poxvirus or papiloma virus expression vector. In some embodiments, the one or more nucleic acids are comprised in a single viral vector. In some embodiments, the one or more nucleic acids are comprised in at least two viral vectors. The neuronal cell may be comprised in a subject. The subject may be a mammalian subject such as, e.g., a primate, monkey, or ape. In some embodiments, the first expressible gene encodes a therapeutic gene product and wherein the subject is a human. The subject may be a mouse. The mouse may be a transgenic, knockout, or knock-in mouse.

Another aspect of the present invention relates to an expression vector comprising h56D (SEQ ID NO: 1), h12R (SEQ ID NO: 3), h56R (SEQ ID NO: 2), h12D (SEQ ID NO: 21), mSST (SEQ ID NO: 4), hPaqR4 (SEQ ID NO: 5), hPaqR4.P3 (SEQ ID NO: 6), Rnf208.1 (SEQ ID NO: 7), or Unc5d.1 (SEQ ID NO: 8), or a complementary nucleotide sequence thereof. In some preferred embodiments, the h56D, h12R, h56R, h12D, mSST, hPaqR4, hPaqR4.P3, Rnf208.1, or Unc5d.1 is operably linked to a promoter or an expressible nucleotide sequence. The h56D (SEQ ID NO: 1), h12R (SEQ ID NO: 3), h56R (SEQ ID NO: 2), h12D (SEQ ID NO: 21), mSST (SEQ ID NO: 4), hPaqR4 (SEQ ID NO: 5), hPaqR4.P3 (SEQ ID NO: 6), Rnf208.1 (SEQ ID NO: 7), or Unc5d.1 (SEQ ID NO: 8) may be in a forward or a reverse position in the vector. The promoter may be a minimal promoter. The minimal promoter may be, e.g., a minimal CMV promoter, a minimal Na/K ATPase promoter, or a minimal Arc promoter. The promoter may be operably linked to a first expressible gene. The first expressible gene and/or the second expressible gene may encode an inhibitory nucleic acid sequence. The inhibitory nucleic acid sequence may be a small interfering RNA (siRNA), a short hairpin RNA (shRNA) or micro RNA (miRNA). The first expressible gene may encode a reporter polypeptide, an ion channel polypeptide, a cytotoxic polypeptide, an enzyme, a cell reprogramming factor, a drug resistance marker, a drug sensitivity marker or a therapeutic polypeptide. In some embodiments, the reporter polypeptide is a fluorescent or luminescent polypeptide.

These techniques (e.g., multi-virus techniques) for accessing key subsets of neurons can provide alternatives to single cell type-specific promoters, and may be used to provide ample protein expression for functional studies, including in vivo imaging and manipulation studies in mammals or in primates, e.g., of the diverse cell populations that comprise the cortex and hippocampus. Indeed, bringing methods that have enabled breakthrough examinations of rodent neural circuit mechanisms to the primate has been a priority for our laboratories. Our techniques can also be combined to further refine cell targeting or used orthogonally in circuit-level experiments. These general methods offer a timely blueprint applicable to many neuron classes and species that will aid the transgenics-independent brain-wide interrogations of functionally significant cell populations.

Yet another aspect of the present invention relates to a host cell comprising an expression vector as described above or herein. The cell may be a bacterial cell. The cell may be a eukaryotic cell. The cell may be a mammalian cell. The cell may be a neuron. The cell may be a cancer cell. In some embodiments, the expression vector is maintained episomally in the cell. In some embodiments, the expression vector is integrated into the genome of the cell. In some embodiments, a single copy of the expression vector is integrated into the genome of the cell.

Another aspect of the present invention relates to a method of assessing the status of a cell comprising: (a) expressing in the cell a vector as described above or herein; and (b) detecting the expression of said first expressible gene and/or said second first expressible gene, thereby assessing the status of the cell. In some embodiments, one of the first expressible gene or the second expressible gene encodes a fluorescent or luminescent polypeptide and wherein detecting the expression comprises imagining the cell to detect expression of the fluorescent or luminescent polypeptide.

In some aspects, an enhancer sequence of h56D or h12R for use in a vector comprising a sequence having at least about 85%, 90%, 91%, 92%, 93%, 94%, 95%, 96%, 97%, 98%, 99% or 100% identity to the sequence h56D or h12R is provided and may be operably linked to a promoter, such as a minimal promoter.

As used herein an “operator element” refers to a DNA sequence that can bind to a polypeptide (also referred to herein as an operator binding element or repressor element), such that the polypeptide affects promoter activity (e.g., the polypeptide can bind to operator element and block transcriptional activity). In some aspects, the operator element is positioned 7-20 nucleotides (e.g., 8, 9 or 10 nucleotides) after the TATA box of the first promoter and/or the second promoter and/or the minimal promoter. In particular, the first promoter and/or the second promoter may comprise a TET, VAN, ETR or OttgR operator element. For example, a first that promoter (such as a hybrid promoter) may be modified to incorporate an operator element.

In some aspects, the vector is a plasmid expression vector or an episomal expression vector. In particular, the vector is a viral expression vector. For example, the viral vector may be a rabies virus (e.g., pseudorabies virus), CAV, adenovirus, adeno-associated virus (AAV), retrovirus, herpesvirus, lentivirus, poxvirus or papilloma virus expression vector. In certain preferred aspects, the vector is an AAV vector, such as an AAV2 vector. In further aspect, the AAV vector comprises ITRs from an AAV2, but coat proteins from a different AAV serotype, such as AAV 1, 5, 7, 8, 9 or an AAV with an engineered coat not found in nature. Combinations of two different viruses or two viruses that have different serotypes may be used in some embodiments to deliver expression plasmids to cells or neurons achieving an additional level of expression restriction.

In another embodiment, two or more viruses may be used to achieve cell type-specific transgene expression that is anatomically restricted. For example, a retrograde viral vector that encodes a recombinase or a repressor from a cell type-specific or a general promoter may be used. The vector may infect neuron axons and axon terminals and can be delivered to a brain or body region that a particular set of neurons innervate. In some embodiments, neurons that carry pain signals from the limbs (here retrograde virus would be delivered to site of pain in a limb), neurons that project from the forebrain to the amygdala and regulate fear (here retrograde virus would be delivered to the amygdala), or neurons that project from the arcuate nucleus to lateral hypothalamus and that regulate hunger (here retrograde virus would be delivered to the lateral hypothalamus) can be targeted using these approaches. A second virus may then be delivered to the site where the neurons originate; for example, the second virus may induce expression of a therapeutic protein, a protein capable of modulating neuron activity, a fluorescent or luminescent protein (e.g., for monitoring neuronal activity from a cell type-specific), or a general promoter, wherein expression requires the presence of a recombinase (because the gene product would otherwise be non-functional). The resulting transgene expression could thus be restricted according to cell type and also according to the location where the cells terminate.

In a further embodiment, there is provided a host cell comprising an expression vector provided herein. For example, the host cell can be a eukaryotic cell, a mammalian cell, a neuron, or a cancer cell. In certain aspects, the expression vector is maintained in the cell as a plasmid or episome. In some aspects, the expression vector is integrated into the genome of the cell. In certain aspects, there is a single copy of the expression vector is integrated into the genome of the cell. In further aspects, the cell comprises 2, 3, 4, 5 or more integrated copies of the vector.

In another embodiment, there is provided a method of assessing the status of a neuronal sub-population comprising: (a) expressing in the cell vectors provided herein; and (b) detecting the expression of said first expressible gene and/or said second first expressible gene, thereby assessing the status of the cell. In some embodiments, one of said first expressible gene or said second expressible gene encodes a fluorescent or luminescent polypeptide and wherein detecting the expression comprises imaging the cell to detect expression of the fluorescent or luminescent polypeptide. In some embodiments, the cell is ex vivo. In other embodiments, the cell is in vivo. The cell may be a mammalian cell, such as a mammalian neuron. In some aspects, one or both of said first promoter or said second promoter comprises operator elements that provide cell type-specific expression in cells of interest. The first promoter and second promoter may preferably contain regulatory elements such as, e.g., TetO, one or more repressors (e.g., TetR), and/or recombinase domains for Cre/Flp/Dre to drive or repress expression of a gene or transgene in cellular or neuronal sub-populations.

In another embodiment, there is provided a method of treating a mis-regulated cell comprising expressing in the cell a vector provided herein, wherein said vector encodes a therapeutic gene product and/or a fluorescent or luminescent polypeptide (e.g., to monitor cell status vis-a-vis activity of therapeutic gene product) and second vector encodes a recombinase or repressor able to alter expression from the first vector to achieve cell type-specific expression of the therapeutic gene product and/or a fluorescent or luminescent polypeptide. In certain aspects, the cell is ex vivo. In other aspects, the cell is in vivo. In some embodiments, the cell is a neuronal cell in a mammalian subject, such as a rodent, a primate, or a human subject.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G: Organization and specificity of candidate GABAergic promoters. Hybrid promoters were constructed using segments of human genomic DNA, that are substantially similar to reciprocal mouse sequences. (A) Human D1×1/2 and D1×5/6 intergenic regions were aligned de novo to mouse genomic DNA. Human genomic segments are shown; black lines depict base pair differences. Enhancers tested in this study are in grey. Enhancers described previously (Dittgen et al., 2004; Ghanem et al., 2003) are in white. Enhancers that were selected for detailed cell type-specific characterization are marked with asterisks. Where applicable, arrowheads indicate the original orientations of the cloned enhancer domains within chromosomal DNA. Scale bar: 500 base pairs. (B) An rAAV construct used to test promoter specificity comprised: a hybrid promoter consisting of an enhancer domain in the 5′ to 3′ orientation with respect to diagram (A) and the cytomegalovirus minimal promoter (CMV MP) is followed by the foreign protein coding sequence, woodchuck hepatitis virus posttranscriptional regulatory element (WPRE), and simian virus 40 polyadenylation sequence, all flanked by AAV2 inverted terminal repeats (ITRs). (C) The rAAV vector h12R-tdTomato was injected into mouse hippocampal area CA1 (upper panels) and cortex (lower panels). Brain sections were analyzed by in situ mRNA hybridization using probes to tdTomato (tdT, red) and to endogenous glutamic acid decarboxylase (GAD65, green) transcripts (insets). Upper panels: A representative image of the injected dorsal hippocampus is shown: h12R is inactive in some GABAergic neurons throughout the hippocampus, but especially in strata radiatum and lacunosum-moleculare. Subsequent analysis indicated that many of the missed cells were NPY⁺ and VIP⁺ (see FIG. 3). (so: stratum oriens, sp: stratum pyramidale, sr: stratum radiatum, slm: stratum lacunosum-moleculare). Lower panels: Representative image of the injected cortex, layer 2/3. DAPI labeling is occasionally absent despite clear mRNA signals, likely when cell nucleus is separated during thin sectioning. Green arrows mark GABAergic cells not labeled by the virus. Scale bars: 200 μm for panel 1, 20 μm for all other panels, including inset. (D) Targeting quantitation, indicated as mean±SE. Mouse HPC: specificity 96.3±1.9%, coverage 83.4±0.8% (n=4 sections, 4 mice, 262 GAD65⁺ cells), Mouse CTX: specificity 92.8±2.0%, coverage 84.0+2.8% (n=4 section, 2 mice, 1418 cells). (E) Cortical panel shows examples of high and low reporter expression from the h12R promoter. Histogram shows the bimodal distribution (Hartigan's Dip Statistic P<0.002) of reporter expression estimated using mean fluorescence intensity as described in Methods. Bin width was 300 fluorescence units; cells below 2000 units intensity were considered weakly expressing. Strongly and weakly labeled populations were evident from the h12R promoter (weak expression: 177, strong expression: 328, 35%; n=5 sections, 2 mice, 505 tdT+ cells). Schematics depict relative expression from each GABAergic promoter: strong expression (red), weak expression (pink), no expression (white), non-GABAergic cells (gray). (F) The rAAV vector h56D-tdTomato was injected into mouse and gerbil hippocampal area CA1 (top and middle panels) and mouse, gerbil and marmoset cortex (bottom panels). Brain sections were analyzed by in situ mRNA hybridization using probes to tdTomato (tdT, red) and to endogenous glutamic acid decarboxylase (GAD65, green) transcripts (insets). Top and middle panels: Representative images of the injected mouse and gerbil dorsal hippocampus showing that all virus-targeted neurons were GABAergic. Bottom panels: Representative images of injected mouse, gerbil and marmoset cortical layers 2/3. Red arrow points to a gerbil virus-targeted cell that was not GABAergic. Scale bars: 200 μm for panel 1, 20 μm for all other panels, including inset. (G) Targeting quantitation, indicated as mean±SE. Mouse HPC: specificity 94.9+1.0%, coverage 91.4±1.1% (n=5 sections, 4 mice, 324 GAD65⁺ cells). Gerbil HPC: specificity 98.4±1.6%, coverage 90.2±4.0% (n=3 sections, 2 gerbils, 85 GAD65± cells) Mouse CTX: specificity 93.1±1.0%, coverage 92.8±1.4% (n=5 sections, 2 mice, 1256 cells). Gerbil CTX: specificity 83.6±0.3%, coverage 96.6±0.4% (n=3 sections, 2 gerbils, 769 cells). Marmoset CTX: specificity 96.5±1.6%, coverage 88.0±1.5% (n=3 sections, 1 animal, 1569 cells). In all cases, specificity refers to the percent tdT⁺ (red) cells that are GAD65⁺, reflecting the cell type-specificity of the targeting vector; coverage is the percent of GABAergic cells that had been labeled.

FIGS. 2A-2B: The h56D promoter supports direct GCaMP6f expression in putative inhibitory neurons of awake behaving primates. (A) Marmoset cortical area MT was injected with rAAV h56D-GCaMP6f. A representative imaging plane 8 weeks post-injection is shown. Scale bar: 50 μm. (B) Responses to visual stimuli for two representative cells circled red and blue in (A) are shown. Bars below each trace mark stimulus presentations: red bars for the preferred stimulus, gray bars for the non-preferred stimulus. The motion direction for each stimulus is indicated by the arrows. Responses were first detected 6 weeks post-injection.

FIGS. 3A-3D: h12R and h56D promoters are differentially active in subclasses of mouse GABAergic interneurons. rAAV vectors h12R-tdTomato and h56D-tdTomato were injected into mouse hippocampal area CA1 (columns 1, 3) and cortex (columns 2, 4). Brain sections were analyzed by in situ mRNA hybridization using probes to tdTomato (tdT, red) and to each of PV, SST, NPY and VIP (green) transcripts. All hippocampal and cortical layers were examined and counted, as in FIG. 1, but only detailed images are shown. (A) First column: representative hippocampal sections indicate that the h12R promoter was active in nearly all PV⁺ and SST⁺ interneurons, but not in all NPY⁺ and VIP⁺ neurons. (so: stratum oriens, sp: stratum pyramidale, sr: stratum radiatum). Second column: representative cortical layer 2/3 sections demonstrate that the h12R promoter was active in nearly all PV⁺ and SST⁺ interneurons, but inactive in some layer 2/3 and layer 5/6 NPY⁺ neurons and in some layer 2/3 VIP⁺ neurons. Green arrows mark missed cells within each class. Orange-boxed insets (green channel omitted) show examples of NPY⁺ and VIP⁺ neurons that were not labeled by the virus (tdT-). (B) First column: representative hippocampal sections indicate that the h56D promoter was active in nearly all neurons of each class. Second column: representative cortical layer 2/3 sections indicate that the h56D promoter was likewise active in nearly all cortical neurons of each class. Blue-boxed inset (green channel omitted) shows that even seemingly green-only VIP⁺ neurons were tdT⁺. (C) Targeting quantitation (coverage) for h12R, indicated as mean±SE, by class and cortical layer. Mouse HPC (n=5 sections, 3 mice per probe) Mouse PV: 97.2±1.8% (69 PV⁺ cells), Mouse SST: 98.0±1.4% (55 SST⁺ cells), Mouse NPY: 75.4±2.0% (108 NPY⁺ cells), Mouse VIP: 77.7±2.7% (24 VIP⁺ cells). Based on these cell counts, the majority of neurons missed by h12R were NPY⁺. Mouse CTX (n=4 sections, 2 mice per probe). Mouse PV: L2/3 98.0±2.0% (1248 cells), L4 94.0±3.6% (1329 cells), L5/6 93.8±3.8% (1255 cells); Mouse SST: L2/3 97.5±2.5% (1132 cells), L4 100±0% (1376 cells), L5/6 95.7±2.6% (1285 cells); Mouse NPY: L2/3 90.3±1.7% (1339 cells), L4 96.8±3.3% (1200 cells), L5/6 73.3±2.0% (1261); Mouse VIP: L2/3 75.3±5.0% (966 cells), L4 100±0% (1353 cells) L5/6 93.8±6.3% (1212 cells). (D) Targeting quantitation for h56D, indicated as mean±SE, by class and cortical layer. Mouse HPC (n=4 sections, 3 mice per probe) Mouse PV: 94.5±1.5% (78 PV⁺ cells), Mouse SST: 94.6±2.0% (62 SST⁺ cells), Mouse NPY: 94.5±1.0% (99 NPY⁺ cells), Mouse VIP: 90.3±1.7% (23 VIP⁺ cells). CTX (n=3 sections, 3 mice per probe) Mouse PV: L2/3 94.3±5.7% (882 cells); L4 97.0±3.0% (1200 cells), L5/6 93.0±3.5% (780 cells); Mouse SST: L2/3 100±0% (796 cells); L4 93.3±6.7% (671 cells), L5/6 94.3±2.9% (802 cells); Mouse NPY: L2/3 97.6±2.4% (791 cells), L4 97.0±3.0% (1148 cells), L5/6 94.4±5.6% (831 cells); Mouse VIP: L2/3 90.8±4.6% (850 cells), L4 100±0% (855 cells) L5/6 100±0% (780 cells). The h56D promoter was active in nearly all GABAergic interneurons of each subclass and across cortical layers. Scale bars: 20 μm throughout.

FIGS. 4A-4D: Set intersection strategy to target somatostatin interneurons in rodent and primate. (A) Sequence conservation between mouse and human genomic DNA at the mouse somatostatin (SST) gene locus. Upstream and downstream non-coding regions (red) show elevated sequence conservation, as indicated numerically at right. Additional more distant conserved domains were detected. ECR Browser (Ovcharenko et al., 2004) settings: domain length 100, similarity cutoff 50. Selected promoter region extends ˜2000 base pairs upstream of the SST start codon, covering three conserved domains. SST mRNA untranslated regions (yellow), exons (blue) and intron (orange) are indicated. (B) Two-virus set intersection strategy: SST-Cre and h56D-(EGFP)^{Cre} viruses are co-injected; EGFP is expressed only when both promoters are active in the same cell. (C) Representative hippocampal sections for mouse and gerbil and cortical layer 2/3 sections for mouse examined using in situ hybridization probes to EGFP (green) and SST (red) transcripts. Cell nuclei were additionally DAPI stained. Marmoset layer 2/3 cortical sections were stained with antibodies against EGFP (green) and SST (red). Red arrow indicates an unlabeled SST⁺ cell. Scale bars: 20 μm throughout. (D) Quantitation of SST neuron targeting in mouse and gerbil hippocampus and mouse and marmoset cortex indicated as mean±SE. Mouse HPC: specificity 92.3±1.5%, coverage 91.3±0.9% (n=4 sections, 3 mice, 43 SST⁺ cells). Mouse CTX: specificity 90.2±1.5%, coverage 87.9±5.6% (n=3 sections, 2 mice, 769 cells). Gerbil HPC: specificity 86.7±2.8%, coverage 97.2±2.7% (n=3 sections, 2 gerbils, 34 SST⁺ cells) Marmoset CTX: specificity 98.5±1.5%, coverage 88.3±2.7% (n=3 sections, 1 animal, 60 SST⁺ cells).

FIGS. 5A-5E: Set intersection strategy to target parvalbumin interneurons in rodent and primate. (A) SArKS-facilitated selection of the PaqR4 gene (Wylie et al., 2018). Transcriptome data (Mo et al., 2015) was filtered based on chromatin accessibility (ATACseq) across neuron classes to identify a subset of mRNA species whose expression was above a set threshold in PV⁺ neurons, but below that threshold in other neuron classes (Wylie et al., 2018). PV⁺, VIP⁺ and excitatory (EXC) neuron rows indicate average log-transformed transcripts per million (TPM) values for the 196 genes meeting these two criteria. Genes were hierarchically clustered based on expression profiles across the three neuron types (as shown in dendrogram). The set was refined, as follows. Remaining rows represent additional filters: (1) the log 2-ratio of gene expression in PV⁺ neurons compared to other neuron types-genes with values >1 (black bars) were retained; (2) PV-versus-other differential expression t-statistic; and (3) SArKS motif-based regression model score. For (2) and (3) black bars mark the top 5% of the 6,326 SArKS-analyzed genes (Wylie et al., 2018). The final row contains 11 genes that remain after all the filters have been applied (black bars) and genes that had been eliminated by the SArKS filter (blue bars); PaqR4 (red bar) is indicated by an arrow. (B) Sequence conservation between mouse and human genomic DNA at the human PaqR4 gene locus. Upstream non-coding region (red) shows elevated sequence conservation, as indicated numerically at right. ECR Browser (Ovcharenko et al., 2004) settings: domain length 100, similarity cutoff 50. Selected promoter region extends ˜800 base pairs upstream of the PaqR4 transcription start site (TSS). PaqR4 and the upstream Kremen2 gene mRNA untranslated regions (yellow), exons (blue) and intron (orange) are indicated. (C) Two-virus set intersection strategy: PaqR4-Cre and h56D-(EGFP)^{Cre} viruses are co-injected. Expression of EGFP can occur if both promoters are active in the same cell. (D) Representative hippocampal sections for mouse and gerbil and cortical layer 4 sections for mouse examined using in situ hybridization probes to EGFP (green) and PV (red) transcripts. Cell nuclei were additionally DAPI stained. Marmoset layer 4 cortical sections were stained with antibodies against EGFP (green) and SST (red). Scale bars: 20 μm throughout. Yellow arrows indicate EGFP⁺/PV⁺ double positive cells, green arrows indicate EGFP⁺/PV⁻ cells, while red arrows point to EGFP⁻/PV⁺ cells. For clarity, not all EGFP⁺/PV⁺ are marked. (E) Quantitation of PV⁺ neuron targeting in mouse hippocampus and mouse and marmoset cortex indicated as mean±SE. Mouse HPC: specificity 79.8±4.9%, coverage 91.3±0.9% (n=5 sections, 3 mice, 86 PV⁺ cells). Gerbil HPC: specificity 76.8±1.3%, coverage 91.4±4.6% (n=3 sections, 2 gerbils, 52 PV⁺ cells). Mouse CTX: specificity 69.1±1.4%, coverage 87.1±3.5% (n=3 sections, 2 mice, 813 cells). Marmoset CTX: specificity 87.4±1.4, coverage: 87.1±3.5% (n=3 sections, 1 animal, 114 PV⁺ cells, 1061 cells).

FIGS. 6A-6B: Set difference strategy to target mouse hippocampal excitatory neurons. Hippocampal excitatory neurons were isolated using the h56D promoter to subtract GABAergic interneurons from all neurons. (A) Schematic demonstrates the set difference strategy. A mix of h56D-Cre and hSYN-(EGFP_{FWD})^{Cre} viruses is injected. In the inhibitory neurons, Cre recombinase shuts off EGFP expression. However, no recombinase is synthesized in excitatory neurons, where the h56D promoter is inactive. In the primary vector EGFP is floxed in the forward orientation, such that it is made in all neurons when Cre recombinase is absent. (B) Brain sections were analyzed by in situ mRNA hybridization using probes to EGFP (green) and to endogenous glutamic acid decarboxylase (GAD65, red) transcripts. Representative section of the injected mouse hippocampal area CA1 following subtraction: Cre-expressing GABAergic interneurons lacked EGFP (88.8±1.0% GAD65⁺ cells were EGFP⁻, n=3 sections, 2 mice, 61 GAD65⁺ cells), while putative stratum pyramidale excitatory neurons continued to express EGFP. Cell nuclei were DAPI stained (blue) to confirm hippocampal layers (so: stratum oriens, sp: stratum pyramidale). Scale bar: 20 μm.

FIGS. 7A-7D: Set difference strategy to target mouse hippocampal NPY⁺ interneurons. (A) A mix of three rAAVs shown in the schematic was injected into NPY-Cre mouse dorsal hippocampus and cortex. hSYN-(EGFP)^{Cre} was used to label endogenous Cre-expressing neurons green. h56D_{TetO4}-tdTomato and h12R-TetR vector mix (h56D/h12R-tdTomato) was used to label virus-targeted neurons red. Double-labeled NPY⁺/tdT⁺ neurons are shown in yellow. (B) Direct reporter fluorescence within a representative dorsal hippocampal section shows that most virus-targeted neurons were NPY⁺, but not all NPY⁺ neurons had been labeled (green arrows). The labeled NPY⁻ cells (red arrows) were VIP⁺. Most of the virus-targeted NPY⁺/tdT⁺ neurons were found in stratum oriens, while fewest were seen in stratum lacunosum-moleculare. (C) Representative sections showing cortical layers 2/3 and 5/6. No virus-targeted cells were observed in layer 4. As in (B), most virus-targeted neurons were NPY⁺, but that not all NPY⁺ neurons had been labeled (green arrows). Red arrows mark NPY⁻/tdT⁺ neurons, which were not characterized. Scale bars: 20 μm. (D) Virus-targeted neuron counts per brain region are plotted as mean±SE. Mouse HPC: specificity (NPY) 89.7±1.3%; coverage (NPY) 63.5±2.3%; so: specificity 93.1±1.0%, coverage 72.6±6.2%; sp: specificity 66.5±4.0%, coverage 54.5±9.9%; sr: specificity 100%, coverage 50.2±10.5%; slm: specificity 100±0%, coverage 27.8±1.6% (n=8 sections, 3 mice, 165 GFP⁺ cells). Mouse CTX: specificity 87.9±1.8%, coverage 44.9±3.5% (n=4 sections, 2 mice, 305 EGFP⁺ cells, >1500 cells total); L2/3 specificity 83.4±1.1%, coverage 35.4±2.3%, (n=2 sections, 2 mice, 107 EGFP⁺ cells); L5/6 specificity 91.4±1.9%, coverage 55.6±6.4%, (n=4 sections, 2 mice, 166 EGFP⁺ cells).

FIGS. 8A-8F: In vivo functional imaging of virus-targeted SST⁺ and NPY⁺ interneurons. Wild type mice were injected with virus mixes to express GCaMP6f in either dorsal hippocampal SST⁺ or NPY⁺ interneurons and head-fixed to facilitate two-photon microscopy while awake and behaving. (A) Representative in vivo two-photon image showing GCaMP6f expressed in SST⁺ neurons in dorsal CA1 stratum oriens. (B) Mice ran on a treadmill while discrete stimuli (10 trials each: air-puff, light, and tone) were presented in pseudorandom order. GCaMP6f fluorescence traces (ΔF/F) for individual SST⁺ neurons in (A). Traces cover ˜300 s session interval. Cells 1, 2, and 3 show persistent responses to the aversive air-puff to the snout; cell 4 does not respond to air-puff. Animal velocity and stimulus presentations are indicated below the traces. (C) Trial averaged-responses of all cells and all trials to discrete stimulus presentations and bouts of extended (>5 s) locomotion as mean with shaded ±SE (n=2 mice, 21 cells). (D) Representative in vivo two-photon image showing GCaMP6f expressed in NPY⁺ neurons in dorsal CA1 stratum oriens. (E) GCaMP6f fluorescence traces (ΔF/F) for the NPY⁺ cells indicated in (D). Traces cover ˜175 s session interval. Animal velocity is indicated below the traces (n=2 mice, 75 cells). (F) The cross-correlation of ΔF/F activities for 26 cells in a single field of view shows distinct groups that are respectively positively (green) and negatively (brown) correlated. GCaMP6f-Ca²⁺ signals from selected ROIs were extracted and processed using the SIMA package (Kaifosh et al., 2014). Scale bars: 25 μm.

FIGS. 9A-9D: Hybrid promoter screen in the rodent brain reveals two promoter candidates for targeting GABAergic interneurons. Mouse dorsal hippocampal area CA1 was injected with the indicated viral vectors. Representative fluorescent protein expression in 50 μm coronal sections is shown. (A) h12R and h12RL promoters display similar reporter expression patterns. Slight differences in oriens versus lacunosum-moleculare staining between the two vectors is due to injection depth variations. Lower panels: co-injected h12R-tdTomato and h12D-EGFP vectors show identical cell labeling patterns. (B) h56iiD/R promoters were inactive in the mouse hippocampus. (C) h56D supported strong reporter expression in putative GABAergic interneurons; h56R promoter supported reporter expression in many CA1 pyramidal neurons as well as in putative GABAergic cells. h12R and h56D promoters were selected for in-depth characterization. (so: stratum oriens, sp: stratum pyramidale, sr: stratum radiatum, slm: stratum lacunosum-moleculare). Scale bars: 20 μm. (D) Mongolian gerbil was co-injected with h56D-tdTomato and hSYN-EGFP in the central nucleus of inferior colliculus (ICC, as indicated in the schematic). Robust expression of EGFP was observed but no evidence of h56D promoter activity. Scale bars: 200 μm for the injection site, 20 μm for insets.

FIGS. 10A-10F: h56D promoter supports direct and intersectional reporter expression in the macaque cortex. Indicated virus mixes were injected at a total of eight cortical sites in two rhesus macaque monkeys. Widefield epifluorescence was first detected 2-5 weeks post-injection. Images were taken 5-8 weeks post-injection. (A-C) Top panels: reference cortical vasculature at each site illuminated at 540 nm. Sites shown in (A) and (C) are near the edge of the chamber, which created a visual artifact (whitening) in the upper right corner of the reflectance images. (A) h56D-tdTomato construct supported reporter expression in putative cortical GABAergic interneurons. Red circle is centered on the injection site; a second injection site is visible above and to the left of the main injection site. (B) EGFP was expressed in putative GABAergic interneurons using an intersectional strategy. hSYN-Cre and h56D-(EGFP)_{Cre} vectors were co-injected, such that reporter expression from the h56D promoter was Cre recombinase-dependent. (C) SST-Cre and h56D-(EGFP)_{Cre} vectors were co-injected, such that reporter expression from the h56D promoter was restricted to putative SST. GABAergic interneurons. Identity of targeted neurons in the macaque was not independently confirmed. Circles centered on injection sites are 6 mm in diameter. (D, E) Rhesus macaque cortical area V1 was injected with h56D-GCaMP6f. Recordings were performed 6-7 weeks post-injection at three cortical sites in two animals. Reference vasculature at one site (D) and widefield signal in response to 4 Hz flashed grating (E) at one example site is shown. In the response map, color indicates amplitude of the 4 Hz FFT component computed at each location. Red squares in (D) and (E) mark a 1×1 mm ROI used for the time course recording. (F) Averaged time course of GCaMP6f response to a 4 Hz flashed grating (100 ms on, 150 ms off) with stimulus presentations marked by gray bars. The recording was performed 7 weeks post-injection. Shaded area around the averaged response trace represents ±SEM over 10 trials. The GCaMP signal did not return to baseline at this stimulus presentation frequency, producing an upward baseline drift. The same phenomenon was observed previously in excitatory neurons using CaMKIIa-GCaMP6f (Seidemann et al., 2016).

FIGS. 11A-11B: Single promoters are unable to target SST and PV neuron subclasses. (A) rAAV SST-EGFP injected alone into the mouse hippocampus labeled SST. and CA1 excitatory neurons. Brain sections were analyzed by in situ mRNA hybridization using probes to EGFP (green) and to endogenous SST (red) transcripts. Yellow arrows point to correctly-targeted SST₊ neurons. (B) rAAV PV-EGFP and PaqR4-EGFP was each injected alone into the mouse hippocampus. Brain sections were analyzed by in situ mRNA hybridization using probes to EGFP (green) and to endogenous PV (red) transcripts. Representative coronal sections indicate that the viral PV promoter labeled both PV₊ and PV₋ cells and that PaqR4 promoter labeling nearly all PV₊ neurons, but also putative glial PV₋ cells. Yellow arrows mark examples of correctly-targeted PV₊ neurons, green arrows point to PV cells labeled by the viruses, and red arrow indicates a PV₊ neuron not labeled by the PaqR4 virus. For clarity, not all missed cells are marked (so: stratum oriens, sp: stratum pyramidale). Scale bars: 20 m

FIGS. 12A-12D: Flp recombinase-dependent set intersection strategy to target SST interneurons. (A) Schematic representation of the set intersection strategy: SST-Flp and h56D-(EGFP)_{Flp} are co-injected, such that labeling occurs only in cells where both promoters are active. (B) PV-Cre; Ai14 mouse hippocampus (PV₊ neurons are red) was injected with the rAAV mix to label SST₊ neurons green. Representative brain section (50 m) demonstrates orthogonal labeling of PV+ and SST+ neurons. The PV-Cre; Ai14 animal displays elevated labeling of stratum oriens cells consistent with previously reported low level of PV expression in a subset of SST. neurons (Hu et al., 2018). Green arrows: SST₊ virus-labeled neurons; red arrows: PV₊ neurons; yellow arrows: double-labeled neurons. Scale bar: 20 μm. (C) rAAVs SST-Flp and h56D-(EGFP)_{Flp} were co-injected into the gerbil hippocampus. A representative brain section (12 m) analyzed using in situ mRNA hybridization with probes to virus-expressed EGFP (green) and endogenous SST (red) shows specific targeting of SST₊ neurons (yellow arrows). Lower cell counts are related to a difference in section thickness. Cell nuclei were DAPI stained (blue) to confirm hippocampal layers (so: stratum oriens, sp: stratum pyramidale). Scale bar: 20 μm. (D) Quantitation of gerbil SST⁺ neuron targeting presented as mean±SE (Gerbil HPC: specificity 94.5±2.8%, coverage 85.7±7.1%, n=3 sections, 2 gerbils, 51 SST₊ cells).

FIGS. 13A-13C; Set difference strategy used to access mouse excitatory and inhibitory neurons. In each instance (A-C) all neurons were infected with hSYN-(EGFP_{FWD})Cre, where EGFP gene was floxed in the forward orientation, such that it was expressed in all neurons where Cre recombinase was absent. Representative brain sections display direct fluorescence resulting from hSYN-(EGFP_{FWD})Cre expression (green) and h56D-tdTomato expression (red), which is included for reference. Construct schematics indicate the injected rAAV mixes. (A) In the absence of Cre recombinase, EGFP was expressed in both the excitatory and the inhibitory cells, which are double-stained. (B) Cre recombinase was expressed in inhibitory neurons where the h56D promoter was active. As a result, EGFP was expressed only in excitatory (tdT-) neurons, showing no overlap between EGFP and tdTomato-labeled cells. (C) Cre recombinase was synthesized in excitatory neurons where the CaMKIIα promoter was active. As a consequence, EGFP was expressed only in inhibitory (tdT+) neurons, resulting in overlapping green/red labeling. Hippocampal layers are indicated (so: stratum oriens, sp: stratum pyramidale). Scale bars: 20 μm.

FIGS. 14A-14B: Neuron co-infection by multiple viruses. Intersectional neuron targeting normally relies on co-infection by two viruses. Green-red co-labeling of inhibitory neurons requiring three viruses is shown. (A) Schematic of the three rAAV mix, h56D-tdTomato, hSYN-Cre and h56D-(EGFP)Cre, injected into wild type mouse hippocampus. Infection by h56D-tdTomato labeled inhibitory neurons red; co-infection by hSYN-Cre and h56D-(EGFP)Cre viruses labeled inhibitory neurons green. (B) Direct fluorescence in two 50 μm hippocampal slices (bregma −1.6 mm and bregma −2.3 mm) from the same brain is shown. Injection was performed as described in Methods (from bregma: AP −2.2 mm, ML+1.5 mm). Co-labeling of 94-96% of neurons is evident at the center and at the margin of the injection site. Green arrows point to the occasional single color neurons. Scale bar: 100 μm.

FIGS. 15A-15D: Set difference strategy to target mouse hippocampal NPY+ interneurons. (A) Dose-dependent gene expression regulation by the tetracycline repressor (TetR) in cultured fibroblasts. Schematic of interdependent constructs, one encoding TetR and the other containing the CMV minimal promoter with a multimerized tetracycline operator and encoding a reporter protein. HEK293 cells were co-transfected with operator and repressor plasmids (molar ratios indicated). Left panel: reporter was expressed in the absence of repressor (reporter on). Right panel: co-expressed TetR blocked reporter expression (reporter off). (B) Differential tdTomato expression from the h56D and h12R promoters in area CA1 stratum oriens. Viral vector mixes injected into NPY-Cre mice labeled the endogenous NPY+ neurons green and the virus-targeted neurons red. Direct fluorescence within representative dorsal hippocampal sections is shown. Diagram below each panel summarizes experimental observations for each promoter. The bar below each panel displays the fraction of stratum oriens GABAergic tdT+ cells (red) that were NPY+ (yellow). Top left panel: Nearly all NPY+ neurons were labeled by the h56D vector and, as expected, not all GABAergic neurons were NPY+ (specificity: 62.3±3.0%, coverage: 97.8±2.6%; n=5 sections, 3 mice, 140 EGFP+ cells). Top right panel: Approximately 25 percent of NPY+ neurons had not been labeled by the h12R virus (green); of the labeled neurons, one half were NPY+, including those weakly labeled (light green) and strongly labeled (yellow) (specificity: 50.6±2.8%, coverage: 77.5±3.0%; n=4 sections, 2 mice, 153 EGFP⁺ cells). The relatively high percentage of NPY+ GABAergic neurons in these panels reflects their overrepresentation in stratum oriens compared to other hippocampal layers. Middle: A schematic of the NPY+ neuron set difference strategy: When TetR is expressed from the h12R promoter, h56D promoter, fitted with a tetracycline operator (TetO4), is blocked in all cells with high h12R activity; in the remaining cells, h56D-dependent expression continues, significantly enriching for the NPY+ interneurons missed by the h12R promoter. This strategy was used by co-injecting h56DTetO4-tdTomato and h12R-TetR vectors to target NPY+ interneurons. Middle panel: The two-virus set difference mix labeled predominantly NPY+ neurons in strata oriens and pyramidale (specificity: 89.7±1.3%, coverage: 63.5±2.3%; n=8 sections, 3 mice, 165 EGFP+cells). (C) In situ hybridization using a probe to VIP (cyan, white arrow) demonstrates that most virus-labeled NPY− neurons in strata pyramidale and oriens are VIP+ (tdT+/NPY−: 72.2±2.8% express VIP; n=3 non-consecutive sections, 2 mice). (D) Direct fluorescence in two 50 μm hippocampal slices (bregma −1.6 mm and bregma −2.3 mm) from the same brain. Injection was performed as described in Methods (from bregma: AP −2.2 mm, ML+1.5 mm). Images were tiled to examine targeting specificity within and across the injection site, including at injection site boundaries. Tiles are presented as individual numbered panels showing virus-labeled and double-labeled cells; associated cell counts are tabulated below the panels. Aggregate targeting specificity and coverage reported in the main text is provided for reference. Sections shown here were not used to obtain aggregate coverage and specificity values. NPY− virus-labeled cells (false positives) are indicated by red arrows. These include VIP+ neurons shown in (C). Hippocampal layers are indicated (so: stratum oriens, sp: stratum pyramidale). Scale bars: 100 μm for main panels, 20 μm for tiled sections.

FIGS. 16A-16E: Virus-targeted mouse NPY+ interneurons segregate into SST+ and PV+ subclasses. (A) Schematic showing rAAV vectors h56DTetO4-tdTomato, h12R-TetR and hSYN-(EGFP)Cre injected into NPY-Cre mice for experiments in panels (B) and (C). Representative immunostained hippocampal sections are shown. (B) Immunostaining for PV: yellow arrows designate virus-targeted PV+ neurons; cyan arrow points a PV+ neuron that was not virus-labeled (PV+ neuron coverage: 44.1±6.7%; 38.3±6.0% of all PV+ neurons (and 86.8% of virus-targeted PV+ neurons) were PV+/NPY+; PV+/NPY+ neuron coverage: 95.0±8.2%; n=3 sections, 2 mice, 54 PV+ cells). (C) Immunostaining for SST: yellow arrows designate virus-targeted SST+ neurons; cyan arrow points to a SST+ neuron that was not virus-labeled. All virus-targeted SST+ neurons were also NPY+, but not all SST+/NPY+ neurons had bee labeled (SST+neuron coverage: 42.6±7.9%, all were SST+/NPY+; SST+/NPY+ coverage: 80.2±8.2%; n=3 sections, 2 mice, 35 SST+ cells). For clarity, not all neurons in each category are marked. (D) Schematic showing rAAV vectors SST-Cre, h56DTetO4-(EGFP)Cre and h12R-TetR injected into wild type mice for experiment in panels (E) to selectively access SST+/NPY+ neurons using an SST and an NPY restriction (set intersection together with set difference). (E) Brain sections were analyzed using in situ mRNA hybridization using probes to EGFP (red, to reserve green channel for the NPY probe), NPY (green) and SST (cyan). Yellow arrows indicate virus-labeled SST+/NPY+ neurons and cyan arrows indicate virus-labeled NPY−/SST+ neurons. Most targeted cells were SST+/NPY+ (specificity 77.1±2.8%, coverage 95.6±2.9%, n=3 sections, 2 mice, 26 NPY+/SST+ cells). Scale bars: 20 μm.

FIG. 17: SArKS analysis of layer-specific promoter candidates. SArKS detection of positively (orange) and negatively (blue) correlating multi-motif domains (MMDs) in human promoters of high scoring (Nc9, Rnf208) and low-scoring (Bach1, Sepsecs) layer 4 genes. Points represent SArKS sequence-smoothed scores and lines represents SArKS spatially-smoothed sequence-smoothed scores. Repetitive regions prone to higher variability in SArKS are colored gray (and were excluded from further analysis). The two positively-correlated genes displayed significant cross-species homology overlapping the MMD regions.

FIG. 18: Cell type-specific targeting of GABAergic interneurons in the rodent and primate neocortex. Novel virus-based promoters were used to access all GABAergic neurons, somatostatin (SST+) and parvalbumin (PV+) inhibitory neuron subclasses. Cell identity was confirmed by in situ hybridization and by immunostaining (marmoset SST and PV). Bottom: human cortical layer 4-specific promoter and layer 4-5-specific promoter support gene expression in the mouse visual cortex (V1). Of the 10 top-scoring human promoters, 4 displayed layer-specific expression in mouse.

FIG. 19: Gene expression from a broadly active h56R promoter is restricted to cortical layer 4 when a CMV enhancer region is included in the promoter. h56R (top), and h56R with CMV enhancer/NRSE (bottom) are shown.

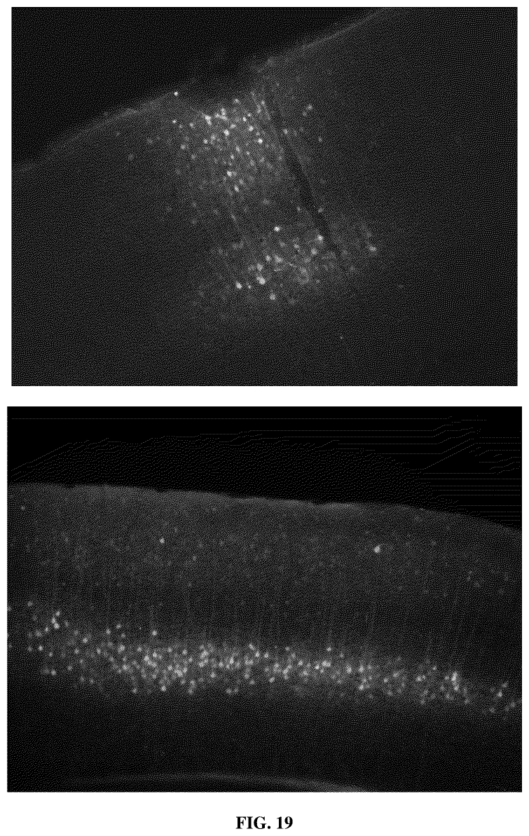

FIG. 20: CCK_{E} neurons comprise 63% of the excitatory ICC population. Nearly all neurons targeted by the viruses were CCK_{E} neurons (specificity: CCK+tdTomato+/tdTomato+, 98.1±2.1%, n=1216 cells, N=5 gerbils; coverage: VGlut2+tdTomato+/tdTomato+, 98.7±0.9%, n=950 cells, N=4 gerbils). The targeted neurons represented ˜75% of CCK+ neurons and ˜50% of excitatory neurons within the 1 mm IC injected site (specificity: CCK+tdTomato+VGlut2+/CCK+VGlut2+, 77.4±5.5%, n=735 cells, N=3 gerbils; coverage: tdTomato+VGlut2+/VGlut2+, 45.8+6.3%, n=950 cells, N=4 gerbils). Example data is shown: brain sections were analyzed using in situ mRNA hybridization using probes to tdTomato (red), endogenous CCK (green), and endogenous VGlut2 (magenta). Filled white arrows mark CCK_{E} neurons labeled by virus (CCK+VGlut2+tdTomato+). Open arrows mark a CCK_{E} neuron not labeled by virus (CCK+VGlut2+tdTomato−). Magenta arrow marks VGlut2+ neuron not labeled by virus (CCK−VGlut2+tdTomato−).

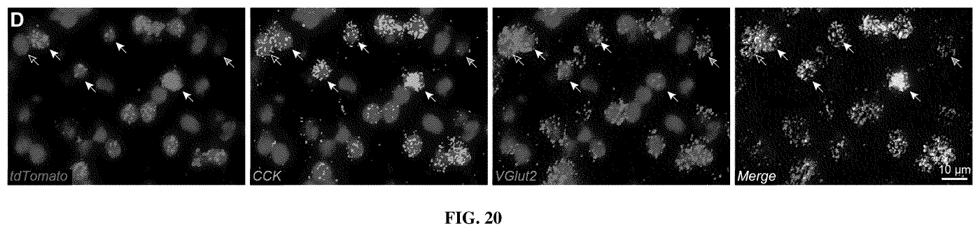

### DESCRIPTION OF ILLUSTRATIVE EMBODIMENTS

The present invention overcomes limitations in the prior art by providing methods and compositions that may be used to induce expression in neuronal sub-populations of a mammal, e.g., in the brain of a rodent or primate. These approaches may be used, e.g., in the generation of genetically modified animals for research, or they may be used in a gene therapy to drive expression in a subset of neurons in a mammalian or primate subject, such as a human patient. In some aspects, hybrid promoters are provided that can be used to drive expression in neuronal sub populations. In some aspects, provided herein are promoters and viral strategies for accessing GABAergic interneurons and their molecularly-defined subsets in the rodent and primate. As shown in the below examples, using a set intersection approach, which relies on two co-active promoters, heterologous protein expression was restricted to somatostatin-positive interneurons. Using an orthogonal set difference method, subclasses of neuropeptide-Y-positive GABAergic interneurons were targeted or enriched by effectively subtracting the expression pattern of one promoter from that of another. These methods can be used significantly expand the number of genetically-tractable neuron classes across mammals. In some embodiments, synthetic enhancers are provided, such as h56D, which may be included in a hybrid promoter to cause expression in particular GABAergic interneurons.

### I. Definitions

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous nucleic acid may be from DNA regions proximate to genes that are not normally active in a sub-populations of neurons, and the exogenous nucleic acid may attain the ability to regulate gene expression in said sub-populations of neurons through change in orientation, a change in sequence, or by being used in neurons of a different species. An exogenous nucleic acid may additionally by a truncated regulatory region that supports transgene expression in different cell types depending on the brain region where it is introduced (for example, using viral delivery), such that the same vector may be active in one class of neurons in the mammalian forebrain, but a different class of neurons in the mammalian brainstem. An exogenous cell may be from a different organism, or it may be from the same organism. By way of a non-limiting example, an exogenous nucleic acid is one that is in a chromosomal location different from where it would be in natural cells or is otherwise flanked by a different nucleic acid sequence than that found in nature. For example, in some embodiments, an exogenous promoter is introduced into a cell, wherein the promoter is from a different species than the cell. As shown in the examples and herein, neuronal promoters from different species can be used to drive expression in neuronal subtypes.

By “expression construct” or “expression cassette” is meant a nucleic acid molecule that is capable of directing transcription. An expression construct includes, at a minimum, one or more transcriptional control elements (such as promoters, enhancers, repressors) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded. In some embodiments, the vector may be linear and single-stranded (e.g., a viral vector).

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, operator regions (that can bind repressors, e.g., TetR), recombinase regions (that can cause encoded gene to be made functional or non-functional), polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence-is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. It may also contain genetic elements at which regulatory proteins such as repressors can bind to block transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. For example, a naturally occurring promoters can be used to drive expression in a cell, and in some embodiments the promoter may be found in or derived from a different species than the species of the cell. In addition, a promoter may enable transgene expression in different cell types depending on the brain region where it is introduced (for example, using viral delivery), such that the same vector may be active in one class of neurons in the mammalian forebrain, but a different class of neurons in the mammalian brainstem. For example, the same h56D promoter sequence is active in the forebrain, in the thalamus, in the olfactory bulb, in the basal ganglia, but not in the brainstem. In some embodiments, the promoter is a synthetic promoter, e.g., containing an enhancer and a minimal promoter element. In some embodiments, the promoter is a synthetic chimeric promoter, e.g., containing domains from multiple related or unrelated or man-made regulatory elements that supports a different gene expression pattern than either of the regulatory elements alone. In some embodiments, the promoter contains a synthetic promoter or an enhancer that is oriented differently than the way it is oriented in nature with respect to the minimal promoter and/or the expressed gene and display different cell specificity than in its original orientation. The orientation of the promoter may affect whether or not gene expression occurs in specific cells or classes of cells. For example, the h56D promoter is active exclusively in GABAergic inhibitory forebrain neurons in one orientation, but in the opposite orientation it is active in both excitatory and inhibitory forebrain neurons. The promoter may contain a heterologous domain (e.g., TetO, etc.) that can affect functionality or the degree of expression induced by the promoter.

By “enhancer” is meant a nucleic acid sequence that, when positioned proximate to a promoter, may increase or decrease transcription activity relative to the transcription activity resulting from the promoter in the absence of the enhancer domain. In some embodiments, the enhancer may confer specificity in expression patterns or may increase expression in particular cell types. The enhancer may alter expression pattern, or may increase or decrease expression in a subset of cells. For example, the h56D promoter contains sequences that are normally not near any gene and would generally be considered enhancers; however, these h56D sequences can also serve as components of a cell type-specific promoter when positioned next to a minimal promoter and a gene, including the feature of orientation sensitivity, wherein promoter specificity is altered when the purported enhanced domain is inverted, which is traditionally a feature of promoters and not enhancers.

By “operably linked” with reference to nucleic acid molecules is meant that two or more nucleic acid molecules (e.g., a nucleic acid molecule to be transcribed, a promoter, and an enhancer element) are connected in such a way as to permit or block transcription of the nucleic acid molecule. “Operably linked” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules. In some embodiments, a chimeric promoter may be used to induce expression in particular cell types, and TetR and recombinases/recombination sites may also be used to control expression. The nucleic acid chains may be connected in different orientations relative to each other to achieve different expression outcomes.

“Identity” refers to the percent of identity between two polynucleotides or two polypeptides. The correspondence between one sequence and another can be determined by techniques known in the art. For example, percent identity can be determined by a direct comparison of the sequence information between two polypeptide molecules by aligning the sequence information and using readily available computer programs.

A “suicide gene” “lethality gene” or “cytotoxic gene” is a nucleic acid coding for a product, wherein the product causes cell death by itself or in the presence of other compounds. The suicide gene may induce apoptosis in the cell. An example of a suicide gene is p53, and other toxins, such as plant toxins (e.g., gelonin) may also be used.

As used herein “prodrug” means any compound useful in the methods of the present invention that can be converted to a toxic product, i.e. toxic to tumor cells. The prodrug is converted to a toxic product by the gene product of the therapeutic nucleic acid sequence (suicide gene) in the vector useful in the method of the embodiments.

### II. Genetically Targeting or Accessing Neuronal Sub-Populations

A variety of combinations of vectors and first and second promoters may be used to selectively induce or repress expression of an expressible gene (e.g., a reporter gene, a gene therapy) in a particular sub-population of neurons. A variety of natural and synthetic promoters, optionally linked to an enhancer, and/or hybrid promoters that induce expression in different neurons in the brain may be used in various embodiments and in combination with the present invention. In some embodiments, the promoter is continuous or discontinuous. The use of either the set intersectional or set difference or set summation approaches may be used, as desired, to induce or repress expression of the expressible gene in a particular neuronal sub-population. It is envisioned that methods provided herein may be used, e.g., to alter the excitatory to inhibitory (E:I) ration of excitement in the brain of a mammalian subject; thus, in some embodiments, methods provided herein may be used to treat a neurological disorder that may benefit from alterations to the E:I ratio such as, e.g., Alzheimer's disease, Huntington's disease, Parkinson's Disease, pain (e.g., neuropathic pain), or epilepsy. Retrograde techniques, including the use of as retrograde viruses, may also be used to target cells; for example, such approaches may be used to target neurons that project to a brain or body region that are excitatory or inhibitory or modulatory. Particular combinations of promoters may be used to drive or repress expression in particular neuronal sub-types. For example, two or more viruses may be used to achieve cell type-specific transgene expression that is additionally anatomically restricted. The vector may a retrograde viral vector that encodes a recombinase or a repressor from a cell type-specific or a general promoter. This vector can infect neuron axons and axon terminals and may be delivered to a brain or body region that a particular set of neurons innervate. Examples can include neurons that carry pain signals from the limbs (here retrograde virus would be delivered to site of pain in a limb) or neurons that project from the forebrain to the amygdala and regulate fear (here retrograde virus would be delivered to the amygdala) or neurons that project from the arcuate nucleus to lateral hypothalamus and that regulate hunger (here retrograde virus would be delivered to the lateral hypothalamus). A second virus may then be delivered to the site where said neurons originate and may express a therapeutic protein, a protein capable of modulating neuron activity, or a fluorescent or luminescent protein for monitoring neuronal activity from a cell type-specific (excitatory, inhibitory, PV, SST, NPY, etc.) or a general promoter (e.g., synapsin, CAG, EF1, CMV hybrid promoter) wherein expression additionally requires the presence of a recombinase because gene product is otherwise non-functional. Resulting transgene expression would then be restricted according to cell type and also according to the location where the cells terminate: excitatory neurons carrying signals from site of pain could specifically accesses and silenced to reduce pain, inhibitory neurons projecting to the site of pain could be accessed and activated to reduce pain; excitatory neurons projecting from the arcuate nucleus to the lateral hypothalamus could be accessed and silenced to reduce feeding.

### A. Neuropeptide-Y-Positive Interneurons

In some embodiments, neuropeptide-Y (NPY) expressing or neuropeptide-Y⁺ (NPY⁺) neurons may be selectively targeted using methods and compositions provided herein. For example, using the set difference methodology described herein with expression of a gene by a hybrid promoter comprising h56D, wherein the expression is repressed by expression by a hybrid promoter comprising h12R, the expression can be selectively induced or limited to particular NPY+ interneurons. In this way, NPY+ interneurons be selectively express a gene, such as for example a reporter gene or a therapeutic gene.

NPY+ interneurons are known to play a role in a variety of diseases. In some embodiments, it is envisioned that altering neuronal activity of NPY+ interneurons may be used to study or treat epilepsy or epileptic seizures, pain management or reducing pain perception (e.g., analgesia), obesity, anxiety or stress, circadian rhythm, addiction (e.g., alcohol abuse or dependence), blood pressure, and/or a sleep disorder (e.g., sleep apnea, sudden acute respiratory syndrome (SARS), etc.). For example and as shown in the below examples, NPY+ interneuron subtypes, such as SST/NPY neurons, may also be selectively targeted for expression of a gene or transgene.

### B. GABAergic Interneurons

In some embodiments, GABAergic interneurons may be targeted using methods provided herein. GABAergic neurons are particularly important in a variety of disease states, and modulation of GABAergic neurons may be used, e.g., in the treatment of epilepsy or in pain management. Activity of GABAergic neurons can be selectively raised to reduce excitatory neuron firing; alternatively, activity of GABAergic neurons can be reduced to increase excitatory neuron firing. Where the activity of a particular subset of GABAergic neurons normally regulates other inhibitory neurons (e.g., vasoactive intestinal polypeptide or VIP neurons, which are present in mammalian brains as well as in the gut), activating such neurons may have the effect of increasing excitatory neuron activity through a reduction of intermediate inhibition (removal of activity block). Each manipulation of GABAergic neuron activity may change the behavioral or physiological state of an experimental subject or human patient. GABAergic interneurons represent less than a quarter of neurons in the mammalian cortex (Meyer et al., 2011), but play key roles in cortical computations (Allen et al., 2011; Caputi et al., 2013; Fuchs et al., 2007). Most GABAergic neurons originate in the medial and caudal ganglionic eminences (MGE and CGE), and then integrate into cortical circuits (Anderson et al., 1997; Lavdas et al., 1999; Marin and Rubenstein, 2001; Wichterle et al., 1999). The fates of MGE and CGE progenitor cells are determined in part by homeobox transcription factors, including Dlx gene products, expressed during embryonic and postnatal development (Cobos et al., 2007; 2005; Long et al., 2009; Stuhmer et al., 2002a; 2002b).

As shown in the below examples, a comparative approach to uncover short enhancer-like sequences interspersed among Dlx genes and conserved across species (Ellies et al., 1997; Ghanem et al., 2003; Sumiyama et al., 2002; Zerucha et al., 2000) resulted in several cell type-specific promoters when these sequences were modifies and combined in ways not found in nature. Aiming for promoter elements that are reciprocally active, can be tested in the rodent, but are likely to function similarly in the primate, mouse and human genomic DNA were aligned and several Dlx domains were identified that were longer than those shared by a broader range of species (Ellies et al., 1997; Ghanem et al., 2003; Sumiyama et al., 2002; Zerucha et al., 2000). For example, h56D is an enhancer that has been transformed into a promoter. rAAVs encoding these putative promoter elements were then engineered and tested, uncovering a subset of human sequences that can support cell type-specific gene expression in both primates and rodents. Thus, DNA or a promoter from one species (e.g., human) can be used to drive a differing or unique expression pattern in cells from or in a second species (e.g., a non-human primate or rodent). Single rAAVs were produced that can access GABAergic neurons broadly and that interdependent (intersectional) viruses can be employed to limit access to specific excitatory and inhibitory subpopulations.

Interestingly, the h56D when operably linked to a promoter, such as a minimal CMV promoter, was able to drive expression in GABAergic interneurons. When the h56D enhancer sequence is inverted again to produce the reverse orientation in the h56R sequence, specificity for GABAergic interneurons was lost. Orientation of the promoter can change specificity; for example the expression pattern of an existing inhibitory promoter may be altered when inserted into a construct in the reverse orientation. Thus, the orientation of a promoter can be used to alter the specificity of the promoter. The h12R promoter can also be used in some embodiments to express a recombinase or transgene in a particular subset or subclass of neurons. The h12R promoter can additionally be used intersectionally with the h56D promoter, with a recombinase or the TetR to limit expression to still other GABAergic subpopulations.

### C. Excitatory Neurons

The targeting of excitatory neurons with viruses can be achieved using a section of the mouse calcium/calmodulin-dependent protein kinase II alpha (CaMKIIα) promoter (Dittgen et al., 2004). However, under certain conditions this promoter may also be active in inhibitory interneurons (Nathanson et al., 2009a; Schoenenberger et al., 2016) and inactive in subsets of cortical excitatory neurons (Huang et al., 2014; Wang et al., 2013; Watakabe et al., 2015). Moreover, there is considerable regional variation in the expression of endogenous CaMKIIα in the rodent and primate brains (Benson et al., 1992; 1991).

Relying on the broad interneuron specificity of the h56D promoter, a two-virus strategy can be utilized for accessing excitatory-only neurons by effectively subtracting the inhibitory interneuron population from all neurons. The set difference strategy is unlike the set intersection approach in that the vectors are not fully interdependent: the primary vector is active until expression is blocked; an inefficient block results in false positives.

For example, a first viral vector can be generated where a floxed reporter protein in the forward (sense) orientation is transcribed from a pan-neuronal human synapsin promoter (SYN-(EGFP_{FWD})^{Cre}) (Borghuis et al., 2011; Schoch et al., 1996). A second vector expressing the Cre recombinase from the h56D inhibitory promoter can be generated. As shown in the below examples, when co-injected into the mouse dorsal hippocampus, the virus-encoded recombinase converted the sense reporter orientation to an antisense orientation only in inhibitory interneurons, and thus restricted reporter expression to excitatory neurons without relying on the CaMKIIα promoter. If GABAergic interneurons account for approximately 10 percent of mouse hippocampal neurons, a false-positive rate for the set difference strategy (that an excitatory cell turns out to be inhibitory) may be no more than 1-2 percent.

### D. Parvalbumin (PV/pvalb) Inhibitory Neurons

Parvalbumin-expressing (PV⁺) interneurons represent another major inhibitory subclass in the mammalian cortex and hippocampus. PV⁺ basket and axo-axonic cells are key regulators of brain rhythms, and they are intimately involved in the microcircuitry of sensory processing, memory formation and critical period plasticity (Klausberger and Somogyi, 2008) Dysfunction of PV⁺ interneurons has been linked to autism and schizophrenia.

As shown in the below examples, the methods provided herein can be used to target PV⁺ interneurons. PaqR4, a member of the progestin receptor family, was identified. When tested alone in the mouse hippocampus, rAAV encoding the human PaqR4 promoter labeled PV+neurons, but also some excitatory and putative glial cells. However, an intersectional approach using h56D to refine labeling, as described herein to target SST⁺ neurons, displayed high specificity for PV⁺ cells in rodent cortex and hippocampus.

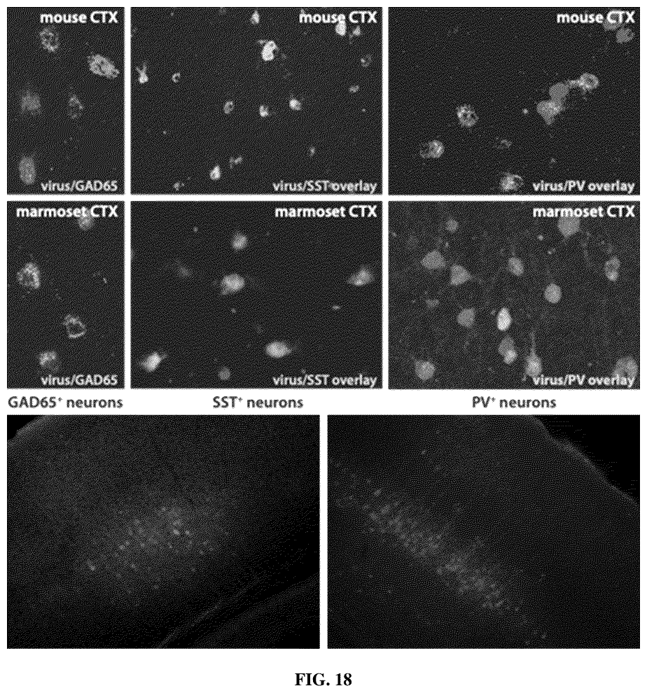

PV+ neurons comprise both basket and chandelier cells. The PaqR4 promoter, which currently targets both neuron subclasses, was altered by deleting each of the four multi-motif domains (MMDs). An initial evaluation indicates that the mix of targeted cells is affected by the combination of MMDs: for example, deletion of the PaqR4 MMD3 reduces the number of SST neurons and increases the number of PV neurons where this engineered promoter is active. Another possibility is to use layer-specific promoters from FIG. 18 that display partial PV specificity. These promoters can be used intersectionally (as described below) with Paqr4 to restrict PV neuron targeting.

### E. NPY Inhibitory Neurons

In some embodiments, neuropeptide-Y (NPY) expressing or neuropeptide-Y⁺ (NPY⁺) neurons may be selectively targeted using methods and compositions provided herein. For example, using the set difference methodology described herein with expression of a gene by a hybrid promoter comprising h56D, wherein the expression is repressed by expression by a hybrid promoter comprising h12R, the expression can be selectively induced or limited to particular NPY+ interneurons. In this way, NPY+ interneurons be selectively express a gene, such as for example a reporter gene or a therapeutic gene.

As shown in the below examples, a set difference strategy can be used to access subsets of NPY⁺ interneurons. These are a diverse population in rodents, both with respect to their origin (Fuentealba et al., 2008; Gelman et al., 2009; Miyoshi and Fishell, 2011; Tricoire and Vitalis, 2012) and function. In addition to modulating individual excitatory neuron firing rates through feed-forward inhibition, NPY⁺ interneurons form gap junctions with each other and nearby GABAergic cells, potentially coupling cortical networks (Armstrong et al., 2012; Fuentealba et al., 2008; Simon et al., 2005). As a neuropeptide, NPY can also promote neurogenesis and acts as an anti-epileptic (Baraban et al., 1997; Noè et al., 2008).

As shown in the below examples, NPY+ interneuron subtypes, such as SST/NPY neurons, which are known to regulate sleep (Kilduff et al., 2011), may also be selectively targeted for expression of a gene or transgene using the methods described herein.

### F. Achieving Cortical Layer-Specific Restriction.

The methods provided herein can be used to target expression in a particular cortical layer. Promoter MMDs can be strongly positively or negatively correlated with layer-specific gene expression. Thus, MMDs can function generally to either enable expression in one or more layers or block expression in all layers except where expression is seen. For example, promoters that show broad or narrow expression specificity can be truncated. An alternative strategy is to rely on generalist promoters, such as CaMKIIα and h56D, by extending them to include the positively or negatively-correlated MMDs. Expression across cortical layers may be observed when using the h56R promoter, but expression from this same promoter may be restricted to cortical layer 4 when a region from the CMV promoter is added to this otherwise broadly-active promoter. It is anticipated that this CMV regulatory region and additional positively or negatively-correlated MMDs may likewise restrict protein expression from cell type-specific promoters.

### III. Expression Constructs

A variety of expression constructs may be used to promote expression in particular neuronal populations. For example, in some embodiments, the construct may comprise an enhancer such as h56D, h56R, h12R, h12D, h12A, SST, or PaqR4 domains, and the enhancer may be operably linked to another regulatory sequence or to a minimal promoter to form a hybrid promoter. It is anticipated that virtually any promoter that causes expression in a population of neuronal cells may be used in various embodiments of the present invention. Although some promoters may induce expression in neuronal cells, this attribute is not required in many embodiments of the present invention. Thus, in some embodiments, the promoter may cause expression in both neuronal and non-neuronal cells. By using the set difference or set intersection approach with genes that express in neuronal and non-neuronal cells, expression of a gene (e.g., a reporter or therapeutic gene) may be limited to particular neuronal cells. In a set summation strategy, two promoters are used to target the sum of the cells that each promoter is able to target alone. In some embodiments, it is anticipated that the compositions and methodologies provided herein may also be used to selectively target non-neuronal cells.

To expand the pool of promoter candidates, sequences that support specific expression (described below) can be fed into a transcriptome mining algorithm (e.g., SArKS, described below) to uncover additional candidate promoters iteratively from transcriptome data. Each validated promoter domain used to seed the search algorithm can generate multiple new promoters. Likewise, each population of mouse or marmoset neurons labeled by a cell-specific promoter represents a starting point for de novo transcriptome and ATACseq studies can yield additional regulatory regions for accessing subsets of labeled cells. Using this strategy, promoter candidates can be defined and tested in the low hundreds. One can then use them intersectionally (as described below), to access key cell classes within marmoset cortical lamina, harnessing overlapping gene expression to restrict cell targeting.

### A. Expressible Genes

The expression construct comprises at least one expressible gene that can be expressed in either direction from the first promoter. In certain aspects, the first expressible gene and/or the second expressible gene encodes an inhibitory nucleic acid, a reporter polypeptide, an ion channel polypeptide, a cytotoxic polypeptide, an enzyme, a cell reprogramming factor, a drug resistance marker or a therapeutic polypeptide. A second promoter is used to express a recombinase, a transposase, or a repressor. Activity by the recombinase, transposase, or repressor can turn on (set intersectional) or turn off (set difference) expression of a functioning version of the expressible gene via a deletion or inversion event. For example, expression of the repressor by a second promoter may silence or repress expression of the expressible gene by the first promoter. In some embodiments, single promoters active in different sub-populations of neurons can be used together to access a larger sub-population of neurons than either promoter alone (“set summation”). Differences in the populations of cells that express the first promoter and the second promoter cause differences in the resulting population of cells that express the functioning version of the expressible gene. Additional promoters may be used with additional repressors and recombinases to further restrict gene expression specificity.

The heterologous protein can be a reporter polypeptide such as, e.g., a fluorescent, bioluminescent, or chemiluminescent protein for labeling and detection of activated cells. Any fluorescent, bioluminescent, or chemiluminescent protein known in the art can be used with the expression construct. A variety of reporter genes can be used which are capable of generating a detectable signal. A variety of reporter genes are contemplated, including, but not limited to Green Fluorescent Protein (GFP), red Fluorescent Protein (mCherry, tdTomato), Blue Fluorescent Protein (BFP), Cyan Fluorescent Protein (CFP), Yellow Fluorescent Protein (YFP), GECIs (genetically-encoded calcium indicators, such as GCaMP6), membrane voltage sensors, pre and postsynaptic neurotransmitter release sensors, presynaptic vesicle release sensors, firefly luciferase, renilla luciferase (RUC), P-galactosidase, CAT (chloramphenicol acetyltransferase), alkaline phosphatase (AP), horseradish peroxidase (HRP), channelrhodopsins, GPCRs, synthetic GPCRs, DREADDs, orthogonal ligands (e.g., to activate or silence neurons), or ionotropic channels (e.g., designed to respond to a ligand not normally found in an organism). Heterologous proteins not already inserted into the cell membrane can be altered to achieve membrane targeting. Heterologous proteins can also be fitted with amino acid signals to target them to neuron axon initial segment, dendrites, axon, cell nucleus, presynaptic compartment, postsynaptic compartment, or mitochondria, etc. The reporter proteins can have degradation signals to alter their half-life such as described in U.S. Patent Publication No. 2004/0146987, incorporated herein by reference.

Additionally, expression constructs can comprise elements of a bipartite system to increase system selectivity and visualize a subset of cells where both promoters are active. One example is a split GFP molecule, where each part is expressed from a different promoter. Both parts must be made in the same cells for fluorescence to be detected. Thus, by expressing the different portions of the split GFP molecule using different first and second promoters, GFP fluorescence can be observed exclusively in cells (e.g., neurons) that drive expression of both the first and second promoter.

In some aspects, the enzyme polypeptide is a recombinase or transposase. For example, the recombinase can be a Cre recombinase, Flp recombinase, Dre recombinase, or Hin recombinase. The expression construct can comprise recombinases (with or without degradation tags and/or regulatory domains), such that the transient recombinase expression will enable or repress constitutive expression of another protein. The recombinases can additionally be regulated by engineered hormone receptor binding domains, such as from human progesterone and estrogen receptors, and activated transiently by the respective ligands that are administered locally or systemically. The expression of recombinases can additionally be regulated by operator elements (such as TetO) inserted between a promoter and the recombinase gene. In this instance, a repressor expressed from the same or different promoter would block recombinase expression. The expression of recombinases can additionally be regulated by other recombinases, where the binding sites for the second recombinase flank or disrupt the first recombinase gene. In this case, the second recombinase would render the first recombinase functionally active or inactive, allowing the targeting methodology to use more than two promoters and thus increasing targeting specificity. In some embodiments, the polypeptide is an activity reporter, repressor, or a neuronal activator or silencer, for example as mentioned above.

In certain aspects, gene for expression in a vector of the embodiments is an inhibitory nucleic acid. For instance, the inhibitory nucleic acid can be an anti-sense DNA or RNA, a small interfering RNA (siRNA), a short hairpin RNA (shRNA) or micro RNA (miRNA). Accordingly, the construct can comprise an RNAi expression cassette. The expression cassette can comprise the coding regions of a gene(s) that is transcribed in vivo to shRNA. The shRNA oligonucleotide design usually comprises a target sense sequence (e.g., a 19-base target sense sequence), a hairpin loop (e.g., 7-9 nucleotides), a target antisense sequence (e.g., a 19-base target antisense sequence) and a RNA Pol II terminator sequence. For example, the hairpin loop can be 5′-TTCAAGAGA-3′ (Sui et al., 2002). The RNA Pol III terminator sequence is usually a 5-6 nucleotide poly(T) tract.

The construct can comprise a lethality or suicide polypeptide such as a cytotoxic polypeptide. A lethality polypeptide is a polypeptide that will cause the cell to expire through apoptosis or necrosis. Generally, a lethality polypeptide could include a toxin polypeptide, an apoptotic cell signal, or a dysregulating event. For example, an exogenous a thymidine kinase (such as from herpes virus) or a protease (e.g., an enzymatically active caspase) gene can be used as the lethality polypeptide. Other cytotoxic polypeptides include, without limitation, gelonin, Caspase 9, Bax, bacterial xanthine/guanine phosphoribosyltransferase gpt, coda, fcyl, a granzyme, Apo-1, AIF, TNF-alpha, or a diphtheria toxin subunit. The construct can comprise a suicide protein to ablate activated cells such as thymidine kinase, nitroreductase, or other enzyme or functional fragment thereof known as applicable for a similar purpose. The coupling product can penetrate into cells which are to be treated with (in the case of thymidine kinase) ganciclovir or another drug (prodrug) of the same family, so that the prodrug is converted in the cells containing the ‘suicide gene’ product to an active form to kill the cells. For example, the suicide gene can be caspase 9, herpes simplex virus, herpes virus thymidine kinase (HSV-tk), cytosine deaminase (CD) or cytochrome P450. Suitable examples of useful known suicide genes and corresponding pro-drugs include thymidine kinase (suicide gene) and ganciclovir/aciclovir (prodrug), nitroreductase (suicide gene) and CB1954 (prodrug), and cytosine deaminase (suicide gene) and 5-fluorocytosine (prodrug). Cytotoxic moieties may be used, e.g., to create animal models of a disease or treat rare brain cancers.

### B. Promoter/Enhancers

A variety of natural and synthetic promoters and enhancers may be used in various embodiments of the present invention. For example, the promoter may cause expression in neuronal cell or be a neuronal promoter such as, e.g., pan-neuronal human or mouse synapsin promoter (SYN), parvalbumin (PV) promoter, somatostatin (SST) promoter, neuropeptide-Y (NPY) promoter, vasoactive intestinal peptide (VIP) promoter, CamKIIalpha, or calbindin. The promoter may be a naturally occurring promoter, derived from a naturally occurring promoter, or a synthetic promoter. The promoter may be continuous or discontinuous. In some embodiments, the promoter is a synthetic promoter such as, e.g., a hybrid promoter. The hybrid promoter may comprise an enhancer such as, e.g., h56D, h56R, h12R, h12D, h12A, mSST, hPaqR4, hPaqR4.P3, Rnf208.1, or Unc5d.1, wherein the enhancer is operably linked to a minimal promoter (e.g., a minimal CMV promoter, a minimal Na/K ATPase promoter, or a minimal Arc promoter). In some embodiments, the promoter causes expression in neuronal and non-neuronal cells. In some preferred embodiments, the promoter includes a neuron specific response element (NSRE) that may reduce or block expression in non-neuronal cell types. A spacer may be used to separate the minimal promoter and enhancer and may be, e.g., 10-200 nucleotides, 20-100 nucleotides, or any range derivable therein. In some preferred embodiments, the promoter can include a regulatory element from cytomegalovirus (CMV) that may limit expression to a particular cortical layer, such as layer 4. A spacer may be used to separate the minimal promoter and enhancer and may be, e.g., 10-200 nucleotides, 20-100 nucleotides, or any range derivable therein.

Promoters are used to drive expression of the expressible genes such as the reporter proteins, recombinases, cytotoxic polypeptides, or a cellular activator or silencer. For example, methods disclosed herein can be used to drive expression in SST, PV, and NPY neurons, or in particular inhibitory cells (e.g., by driving expression in inhibitory neurons and then subtracting expression using the SST, PV, and NPY promoters, to leave expression in inhibitory neurons that are not associated with a particular promoter). A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements can be used to regulate the frequency of transcriptional initiation. Typically, these are located in the region 30-110 bp upstream of the start site, although a number of promoters have been shown to contain functional elements downstream of the start site as well. In certain aspects, the promoter is positioned about 10 to 200 nucleotides, such as 20 to 100 nucleotides, from the expressible gene. In some embodiments, the promoter is an activity-dependent promoter, such as a CRE. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, and in some embodiments promoter function can be preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to additional cis-acting regulatory sequence, e.g., as described herein or that is involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment (e.g., a promoter from a species that is different from the species associated with the cellular environment). A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the P-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB) could also be used to drive expression. Use of a T3, T7 SP6, h56D, h56R, h12R, h12D, h12A, SST, or PaqR4 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Quitsche et al., 1989), GADPH promoter (Alexander et al., 1988), metallothionein promoter (Welch et al., 1989); and concatenated response element promoters, such as cyclic AMP response element promoters (CRE), serum response element promoter (SRE), phorbol ester promoter (TPA) and response element promoters (TRE) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007).

Tissue-specific promoter may be desirable as a way to identify particular cell populations (e.g., neuronal sub-populations). Cell type-specific enhancers can be used to narrow the range of cells in which stimulation will trigger protein expression. To increase both specificity and activity, the use of cis-acting regulatory elements has been contemplated. For example, a neuron-specific promoter may be used. In particular, the promoter is for synapsin I, calcium/calmodulin-dependent protein kinase II, tubulin alpha I, neuron-specific enolase or platelet-derived growth factor beta chain.

In certain aspects, methods of the invention also concern enhancer sequences, i.e., nucleic acid sequences that increase a promoter's activity and that have the potential to act in cis (e.g., regardless of their orientation), even over relatively long distances (up to several kilobases away from the target promoter). However, enhancer function is not necessarily restricted to such long distances as they may also function in close proximity to a given promoter. As described herein, reversing the orientation of the promoter may also be used to alter expression patterns or strength of expression.

### C. Gating Elements

There are several bacterial transcriptional regulators known in the art that can be used with the expression construct of the present invention. The construct can comprise a ligand-inducible or ligand-repressible gating element. Several constructs are available for expressing gates at different levels. In some constructs, the gates have been modified with an additional transcriptional repressor domain to enhance gating. For example, the gates can comprise humanized versions of TetR, MphR, TtgR and VanR bacterial proteins along with their respective DNA binding sites; the ligands of which are doxycycline, erythromycin, phloretin and vanillic acid, respectively. Thus, the expression construct would comprise the DNA binding sites for the bacterial repressor proteins such as a TetO or ETR element. The repressors can be TetR homologs such as AcrR, AmtR, ArpA, BM3R1, BarA, Betl, EthR, FarA, HapR, HlyllR, IcaR, LmrA, LuxT, McbR, MphR, MtrR, PhlF, PsrA, QacR, ScbR, SmcR, SmeT, TtgR, TylP, UidR, or VanR. The operator sequences recognized by the TetR homolog repressors have been previously identified. These operators range 16-55 bp in length, and typically contain inverted repeat sequences.

As described herein and as shown in the examples, inversion of a nucleic acid sequence by a recombinase (e.g., Cre, Flp, or Dre recombinase) may be used to drive or suppress expression of a coding sequence, gene, or transgene by the nucleic acid sequence. In some embodiments, sites at which each recombinase is active to break and rejoin DNA are positioned in a head-to-head orientation flanking a gene that is in an inverted (off) orientation with respect to the promoter. Recombinase appropriate for the recombination sites can then rotate the gene into the forward (on) orientation, activating gene expression. When the gene is originally in the forward (on) orientation, the same activity by the recombinase can inactivate gene expression. In some instances, sites at which each recombinase is active to break and rejoin DNA are positioned in a head-to-tail orientation flanking a gene that is in a forward (on) orientation with respect to the promoter. Recombinase appropriate for the recombination sites will then delete the gene and terminate gene expression.

### D. Vectors

One of skill in the art would be well-equipped to construct the vector through standard recombinant techniques. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors (e.g., an AAV2/1 vector), retrograde AAV vectors, CAV vectors, rabies and pseudorabies vectors, herpes virus vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

### 1. Viral Vectors

Viral vectors may be provided in certain aspects of the present invention. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

In some embodiments, constructs encoding the first and second promoters may be delivered in a single vector in a single virus. In some embodiments, constructs encoding the first and second promoters may be delivered in separate vectors in a different viruses (of the same or different type). The capacity of a given virus to deliver particular amounts of genetic material would of course be taken into consideration when making this decision. In some embodiments, the first and second promoters are delivered to a cell in separate vectors, each contained within an AAV virus. In some embodiments, the first and second promoters may be contained within a retrograde AAV virus. In some embodiments, a vector is transfected into cells, e.g., using a rabies virus, a chicken anaemia virus (CAV virus), pseudorabies, or an AAV virus modified for retrograde transfer.

Retroviruses have promise as gene delivery vectors due to their ability to integrate their genes into the host genome, transfer a large amount of foreign genetic material, infect a broad spectrum of species and cell types, and be packaged in special cell-lines.

In order to construct a retroviral vector, a nucleic acid is inserted into the viral genome in place of certain viral sequences to produce a virus that is replication-defective. In order to produce virions, a packaging cell line containing the gag, pol, and env genes-but without the LTR and packaging components—is constructed. When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium. The medium containing the recombinant retroviruses is then collected, optionally concentrated, and used for gene transfer. Retroviral vectors are able to infect a broad variety of cell types. However, integration and stable expression require the division of host cells.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

### 2. Episomal Vectors

The use of plasmid- or liposome-based extra-chromosomal (i.e., episomal) vectors may be also provided in certain aspects of the invention. Such episomal vectors may include, e.g., oriP-based vectors, and/or vectors encoding a derivative of EBNA-1. These vectors may permit large fragments of DNA to be introduced unto a cell and maintained extra-chromosomally, replicated once per cell cycle, partitioned to daughter cells efficiently, and elicit substantially no immune response. In some embodiments, the episomal vector may be derived from a rabies virus, a chicken anaemia virus (CAV virus), pseudorabies, or an AAV virus modified for retrograde transfer.

In particular, EBNA-1, the only viral protein required for the replication of the oriP-based expression vector, does not elicit a cellular immune response because it has developed an efficient mechanism to bypass the processing required for presentation of its antigens on MHC class I molecules. Further, EBNA-1 can act in trans to enhance expression of the cloned gene, inducing expression of a cloned gene up to 100-fold in some cell lines. Finally, the manufacture of such oriP-based expression vectors is inexpensive.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); Herpes virus saimiri (HS) and Marek's disease virus (MDV). Other sources of episome-base vectors are also contemplated, such as yeast ARS, adenovirus, SV40, or BPV.

Vectors can also comprise other components or functionalities that further modulate gene delivery and/or gene expression, or that otherwise provide beneficial properties to the targeted cells. Such components may be modifications of the viral envelope (capsid). Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also may include markers, such as detectable and/or selection markers that can be used to detect or select for cells that have taken up and are expressing the nucleic acid delivered by the vector. Such components can be provided as a natural feature of the vector (such as the use of certain viral vectors that have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### 3. Transposon-Based System

In certain aspects, the delivery of the expressible gene can use a transposon-transposase system. For example, the transposon-transposase system could be the well-known Sleeping Beauty, the Frog Prince transposon-transposase system (for a description of the latter, see, e.g., EP1507865), or the TTAA-specific transposon PiggyBac system.

Transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

In particular embodiments, the constructs (e.g., the multi-lineage construct) provided in the present invention use a PiggyBac expression system. PiggyBac (PB) DNA transposons mobilize via a “cut-and-paste” mechanism whereby a transposase enzyme (PB transposase), encoded by the transposon itself, excises and re-integrates the transposon at other sites within the genome. PB transposase specifically recognizes PB inverted terminal repeats (ITRs) that flank the transposon; it binds to these sequences and catalyzes excision of the transposon. PB then integrates at TTAA sites throughout the genome, in a relatively random fashion. For the creation of gene trap mutations (or adapted for generating transgenic animals), the transposase is supplied in trans on one plasmid and is co-transfected with a plasmid containing donor transposon, a recombinant transposon comprising a gene trap flanked by the binding sites for the transposase (ITRs). The transposase will catalyze the excision of the transposon from the plasmid and subsequent integration into the genome. Integration within a coding region will capture the elements necessary for gene trap expression. PB possesses several ideal properties: (1) it preferentially inserts within genes (50 to 67% of insertions hit genes) (2) it exhibits no local hopping (widespread genomic coverage) (3) it is not sensitive to over-production inhibition in which elevated levels of the transposase cause decreased transposition 4) it excises cleanly from a donor site, leaving no “footprint,” unlike Sleeping Beauty.

### 4. Other Regulatory Elements

a. Initiation Signals and Linked Expression

A specific initiation signal also may be used in the expression constructs provided in the present invention for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements or protease 2A/cleavage sites are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described (Pelletier and Sonenberg, 1988), as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

b. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

c. Selection and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### E. Delivery of the Expression Constructs

Introduction of a nucleic acid, such as DNA or RNA, into the host cells may use any suitable methods for nucleic acid delivery for transformation of a cell, as described herein or as would be known to one of ordinary skill in the art. Such methods include, but are not limited to, direct delivery of DNA such as by ex vivo transfection, by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (U.S. Pat. No. 5,789,215, incorporated herein by reference); by electroporation (U.S. Pat. No. 5,384,253); by calcium phosphate precipitation; by using DEAE-dextran followed by polyethylene glycol; by direct sonic loading; by liposome mediated transfection and receptor-mediated transfection; by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake, and any combination of such methods. Through the application of techniques such as these, organelle(s), cell(s), tissue(s) or organism(s) may be stably or transiently transformed.

In certain aspects, bidirectional expression constructs of the embodiments are comprised in viral vectors, such as an AAV vector. Thus, in some aspects, the vectors can be delivered to target cells by transducing the cells with the viral vector itself.

### 1. Liposome-Mediated Transfection

In a certain embodiment of the invention, a nucleic acid may be introduced to the host cell by liposome-mediated transfection. In this method, the nucleic acid is entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated is a nucleic acid complexed with Lipofectamine (Gibco BRL) or Superfect (Qiagen). The amount of liposomes used may vary based upon the nature of the liposome as well as the cell used, for example, about 5 to about 20 g vector DNA per 1 to 10 million of cells may be contemplated. In some embodiments, jetPEI® may be used for gene delivery to cells (e.g., adherent cells or cells in suspension).

Liposome-mediated nucleic acid delivery and expression of foreign DNA in vitro has been very successful. The feasibility of liposome-mediated delivery and expression of foreign DNA in cultured chick embryo, HeLa and hepatoma cells has also been demonstrated.

In certain embodiments of the invention, a liposome may be complexed with a hemagglutinating virus (HVJ). This has been shown to facilitate fusion with the cell membrane and promote cell entry of liposome-encapsulated DNA. In other embodiments, a liposome may be complexed or employed in conjunction with nuclear non-histone chromosomal proteins (HMG-1). In yet further embodiments, a liposome may be complexed or employed in conjunction with both HVJ and HMG-1. In other embodiments, a delivery vehicle may comprise a ligand and a liposome.

### 2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also, the amount of vectors used may vary upon the nature of the cells used, for example, about 5 to about 20 g vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### IV. Methods of Use

### A. Detection or Targeting of Activated Cells

In some embodiments, the present invention provides a method of assessing the status of a cell by expressing the expression vector in a host cells and detecting the expression of the first and/or second expressible gene to determine the status of the cell. Detection of the expressible gene can comprise using an instrument selected from the group consisting of a microscope, a luminometer, a fluorescent microscope, a confocal laser-scanning microscope, and a flow cytometer. Cells may be assessed using a sensor of activity, such as GCaMP, etc.

The expression construct provided herein can also be used to target a dysregulated or aberrant cell by expressing the construct in a host cell such that the first and/or second expressible gene encodes a therapeutic or cytotoxic gene product.

The expression construct can be administered to the cell in vivo or ex vivo, and the host cell can be a bacterial, eukaryotic, mammalian, neuron or cancer cell. In certain aspects, the expression construct is administered in combination with a ligand for the gating element such as doxycycline, erythromycin, phloretin or vanillic acid.

### 1. Nervous System

In some embodiments, the expression constructs of the present invention can tag neurons activated during cognitive and physiological states, including fear, hunger, pain, depression, anxiety, addiction, as well as those affected by disease, such as stroke (or other brain injury), neurodegeneration and epilepsy. Tagging neurons, for example those in the brain supporting focal epilepsies, or those degenerating at the onset of Alzheimer's and similar diseases, or those in the peripheral or central nervous system supporting chronic pain, enables such neurons to be visualized and eliminated using traditional imaging and surgical techniques, while sparing nearby healthy neurons.

Alternatively, neuronal tagging during recovery from stroke, other brain injury, or peripheral neuron injury could aid in monitoring healing. In addition, neurons tagged in animal models of human diseases can be isolated and used to screen compound libraries for the ability to selectively alter the function tagged neurons, but not healthy neurons; candidate drugs emerging from such screens could then be tested in human subjects.

In certain embodiments, tagging of neurons activated by candidate drugs administered to experimental animals or human subjects in clinical trials could establish and refine the complement of cells those drugs target, enabling more specific and more personalized treatments to be developed.

Particular brain diseases include brain tumors, Alzheimer's disease, Parkinson's disease, Huntington's disease, lateral amyotrophic sclerosis, neurodegenerative and neurometabolic disorders, chronic brain infections (e.g. HIV, measles, etc.), pituitary tumors, spinal cord degeneration (both inherited and traumatic), spinal cord regeneration, autoimmune diseases (e.g. multiple sclerosis, Guillain Barre syndrome, peripheral neuropathies, etc.) and any other diseases of the brain known to persons skilled in the art.

### 2. Cancer

In some embodiments, specific sub-populations of cells may be targeted that may include cancerous cells. Transformed cells labeled using the methods described herein can be harvested and genetically profiled. In this case the sampled cell population need not be homogeneous, as would be true for advanced tumors, but can include intermixed healthy and transformed cells, since reporter is selective for transformed cells. Detailed information about transformed cell phenotype at an early stage of the disease may aid treatment selection and improve its efficacy. If coupled to activity-dependent promoters, specific transformed cell classes may be selectively targeted.

When the reporter is functionally linked to an enzyme or toxin subunit that can eliminate cells in which it is expressed, the reporter can be a vehicle for highly selective gene therapy. The DNA can be delivered locally using viruses, lipids or any other effective means for getting foreign DNA and RNA into cells, including in an ointment for treatment of skin disorders. Unlike existing treatments that may be toxic to a variety of healthy and compromised cells, the reporter system can be tuned to eliminate diseased cells with minimal impact on nearby healthy cells.

Exemplary cancer cells that can be detected or targeted by the methodologies include brain cancers, such as glioma or glioblastoma multiforme (GBM).

### 3. Block or Enhance Specific Memories

SST neurons have been implicated in fear learning (Lovett-Barron et al., 2014). Activation or silencing of these neurons during memory formation may determine if a memory is formed or blocked. PV neurons have been implicated in working memory (Murray et al., 2011).

### 4. Anxiety

PV neurons in the amygdala are known to regulate expression anxiety and fear (Ehrlich et al., 2009). Modulating the activity of these neuron subclasses could be effective in individual patients to treat memory dysfunction, including inappropriate fear memories, such as PTSD.

### 5. Breathing

SST neurons in the preBötzinger complex are known to serve as a pacemaker for involuntary breathing during sleep (Tan et al, 2008). Modulating the activity of these neurons could be effective in individual patients to eliminate sleep apnea, and to monitor and rescue SST neuron function to prevent SIDS.

### 6. Schizophrenia

SST, PV and NPY inhibitory neuron dysfunction has been implicated in different aspects of schizophrenia (Lewis et al., 2005). Modulating the activity of these neuron subclasses may be used to treat this disease.

### 7. Sleep

NPY+ interneurons are known to play a role in a variety of diseases. In some embodiments, it is anticipated that altering neuronal activity of NPY+ interneurons may be used to study or treat epilepsy or epileptic seizures, pain management or reducing pain perception (e.g., analgesia), obesity, anxiety or stress, circadian rhythm, addiction (e.g., alcohol abuse or dependence), blood pressure, and/or a sleep disorder (e.g., sleep apnea, sudden acute respiratory syndrome (SARS), etc.).

### 8. Pain/Itch

Local and ascending SST and NPY neurons in the spinal cord regulate the perception of pain and itch (Bourane et al., 2015; Pan et al., 2019; Christensen et al., 2016). Chronic and transient pain and itch can therefore be regulated by specifically accessing and modulating the function of these neurons in human patients.

9. Harvesting Specific Neurons for Implantation into Patients

Specific neuron subclasses, such as SST and PV neurons, are known to be lost in neurological disorders, such as schizophrenia and Alzheimer's disease. The ability to identify, label and isolate these neuron subclasses can be used in methods to transplant specific or selected neurons into affected patients.

### B. Administration

The constructs described herein may be administered in any suitable manner known in the art. For example, the constructs may be administered sequentially (at different times) or concurrently (at the same time). In some preferred embodiments, the vector(s) encoding the first and second promoters are injected (e.g., using stereotaxic methods) into the brain, spine, or cerebrospinal fluid at substantially the same time, within a matter of minutes, or within 1-3 hours or less. The vector (e.g., a vector containing h56R) may be administered by the same route of administration or by different routes of administration such as intravenously, intramuscularly, subcutaneously, topically, orally, transdermally, intraperitoneally, intraorbitally, by implantation, by inhalation, intrathecally, intraventricularly, or intranasally. In some embodiments, retrograde viruses or viral variants containing one or more vector as described herein is administered to a subject.

Pharmaceutical compositions and formulations of the constructs of the present invention can be prepared by mixing the active ingredients (such as a nucleic acid or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### C. Test Compound Screening

The methods and compositions provided herein can be used to screen for factors (such as solvents, small molecule drugs, peptides, and polynucleotides) or environmental conditions (such as culture conditions or manipulation) that affect the characteristics of activated or aberrant cells.

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015). In certain aspects of this invention, cells programmed to the hematopoietic lineage play the role of test cells for standard drug screening and toxicity assays, as have been previously performed on hematopoietic cells and precursors in short-term culture. Assessment of the activity of candidate pharmaceutical compounds generally involves combining the hematopoietic cells or precursors provided in certain aspects of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on hematopoietic cells or precursors, or because a compound designed to have effects elsewhere may have unintended effects on hematopoietic cells or precursors. Two or more drugs can be tested in combination (by combining with the cells either simultaneously or sequentially), to detect possible drug-drug interaction effects.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Functional Access to Neuron Subclasses in Rodent and Primate

The present studies concern targeting GABAergic interneurons, which represent less than a quarter of neurons in the mammalian cortex (Meyer et al., 2011), but play roles in cortical computations (Allen et al., 2011; Caputi et al., 2013; Fuchs et al., 2007). To identify promoters active across GABAergic neurons, the present studies focus on the conserved enhancer-like sequences interspersed among Dlx homeobox transcription factor genes that are expressed in interneurons during embryonic and postnatal development (Cobos et al., 2007; 2005; Long et al., 2009; Stühmer et al., 2002a; 2002b). Aiming for promoter elements that are reciprocally active, i.e. can be tested in the rodent, but are likely to function similarly in the primate, mouse and human genomic DNA were aligned to uncover several Dlx domains that were longer than those shared by a broader range of species (Ellies et al., 1997; Ghanem et al., 2003; Sumiyama et al., 2002; Zerucha et al., 2000). AAVs encoding two of these human sequences were broadly active in primate and rodent GABAergic interneurons.

To identify promoters for subclasses of GABAergic neurons, conserved domains were searched for within regulatory regions of genes that are enriched in the respective cell populations. This effort yielded a promoter for targeting somatostatin-positive neurons and another for targeting parvalbumin-positive neurons in the primate and rodent.

The present studies demonstrated that single rAAVs can access cortical and hippocampal GABAergic neurons broadly and that interdependent viruses can be employed to limit access to specific excitatory and inhibitory subpopulations. The results suggest that the general strategy of finding DNA sequences that are conserved between rodent and primate and of relying on combinatorial methods to refine genetic targeting is applicable to many neuron classes and will aid the transgenics-independent brain-wide interrogations of functionally significant cell populations.

Targeting GABAergic Neurons in the Rodent and Primate with Single AAVs:

From the outset, the goal had been two-fold: to assemble short GABAergic interneuron-specific promoters that could be used in viruses, and to maintain promoter specificity across mammalian species, especially in primates, where genomic manipulations can be especially cumbersome. The developmental fate of forebrain interneurons in many species is partly determined by the products of Dlx genes (Cobos et al., 2005; 2007; Long et al., 2009; Stuhmer et al., 2002a; 2002b), the vertebrate counterparts of the D. melanogaster distal-less homeobox proteins. Dlx1-6 genes are arranged in bigene clusters interrupted by intergenic regions that contain highly conserved enhancer-like domains, each several hundred base pairs in length (Ellies et al., 1997; Ghanem et al., 2003; Sumiyama et al., 2002; Zerucha et al., 2000). Rodent and zebrafish variants of these domains incorporated into transgenic mice (Ghanem et al., 2003; Potter et al., 2009; Stuhmer et al., 2002b) have previously been shown to support reporter expression in GABAergic interneurons. In addition, two recent studies described the first interneuron-specific viral vectors containing similar regions (Dimidschstein et al., 2016; Lee et al., 2014).

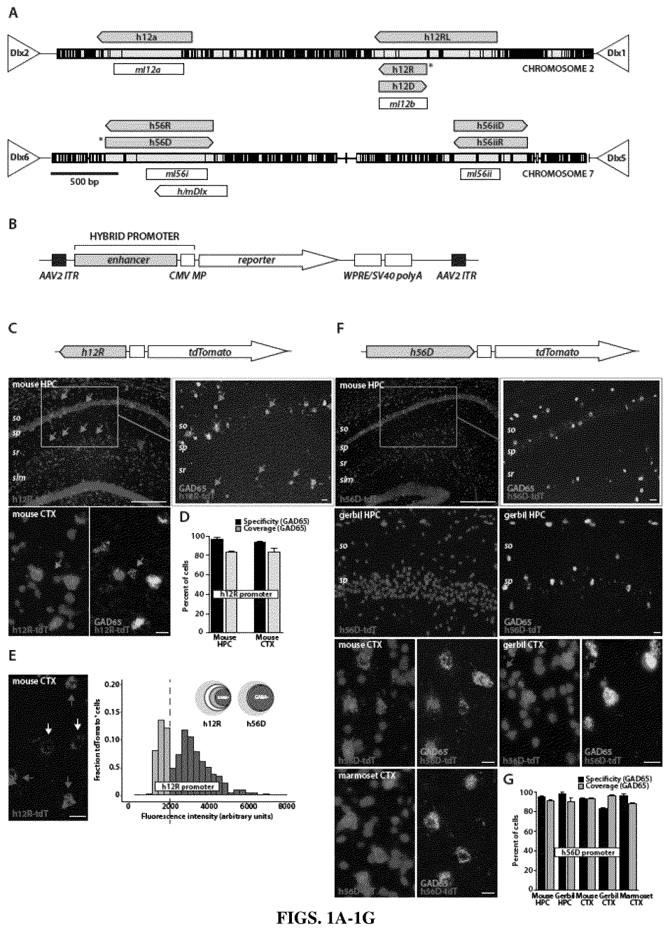

Striving to develop promoters that are likely to be active in the primate brain, but could be initially tested in the rodent, human and mouse Dlx1/2 and Dlx5/6 genomic DNA were aligned de novo and highly conserved reciprocal domains were identified that were longer than those described previously (FIG. 1A). Hybrid promoters were constructed by pairing each enhancer domain with a cytomegalovirus minimal promoter. The resulting regulatory sequences were incorporated into rAAV vectors encoding fluorescent reporter proteins (FIG. 1B) and were initially evaluated for expression strength and specificity in the mouse hippocampus and cortex.

Three human sequences were tested from Dlx1/2; as in previous reports (Ghanem et al., 2003), all were in the reverse orientation with respect to their placement within chromosomal DNA. A promoter containing the human variant of the ml12a domain, h12a (FIG. 1A), labeled mostly inhibitory interneurons, but also some excitatory cells, and was not characterized further.

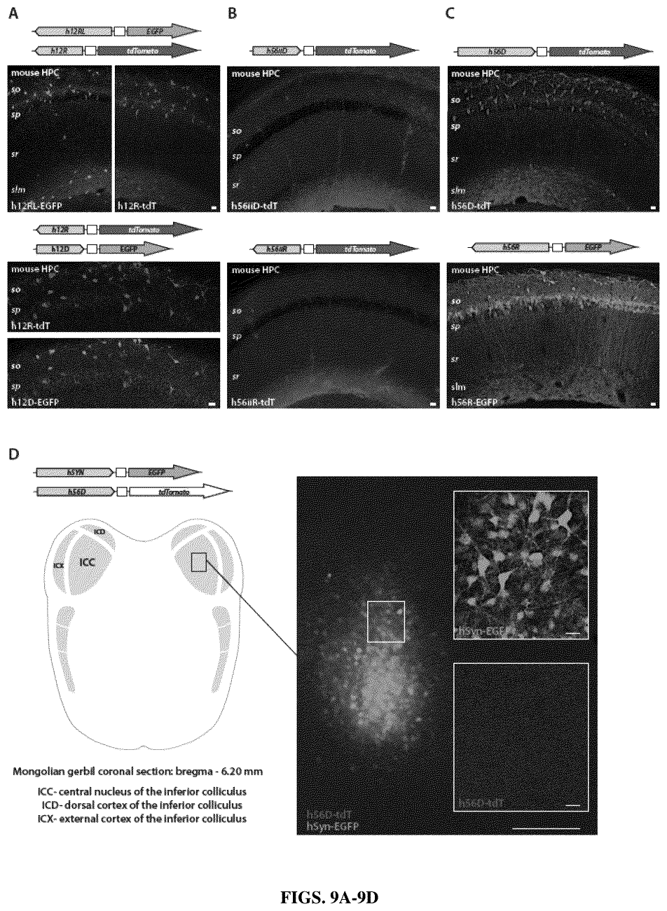

Two promoters incorporating human domains from the Dlx1/2-ml12b region (Ghanem et al., 2003) were also tested: the longer one, the 1000 base pair h12RL, covered the full extent of the human/mouse sequence conservation; the shorter 376 base pair sequence, termed h12R, aligned more closely with the core conserved region at this genomic location (Ghanem et al., 2003), FIG. 1A). Both promoters supported reporter expression in similar numbers of cells. Likewise, expression pattern for each promoter in the mouse hippocampal area CA1 was broadly consistent with successful GABAergic interneuron targeting: most labeled cells were located in stratum oriens, while only a few appeared in stratum pyramidale (FIG. 9A). Based on these initial observations, it was concluded that the significantly longer h12RL did not confer a clear cell type-specific expression benefit.

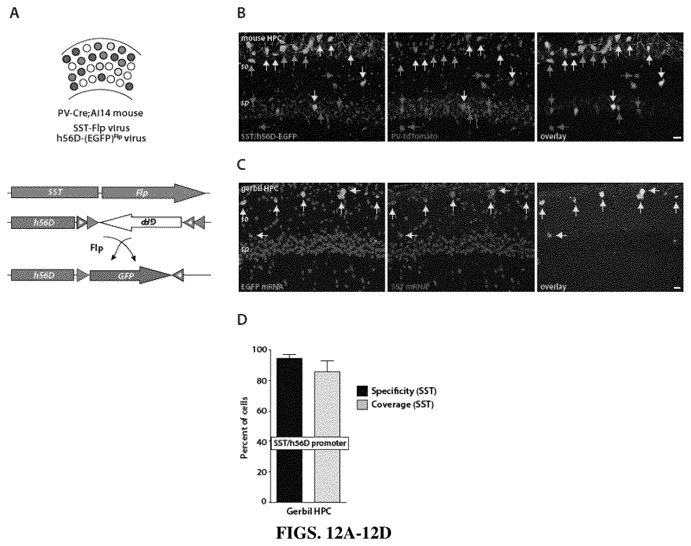

The shorter h12R promoter in mouse cortex and hippocampus was then characterized. Promoter properties were not affected by enhancer orientation, as judged by injecting a mix of two viruses encoding different color reporters (h12R-tdTomato, h12D-EGFP) into the mouse dorsal hippocampus (FIG. 9A). More generally, this experiment demonstrated the high likelihood of individual neuron co-infection by multiple viruses, a feature that was confirmed in follow-up experiments (FIG. 12). The strength and specificity of the h12R promoter was then examined using in situ probe hybridization to reporter mRNA and it was confirmed that it was active predominantly in rodent GABAergic interneurons (HPC: 96.3±1.9%; CTX: 93.2±0.9% of labeled neurons were GABAergic). However, not all GABAergic interneurons had been labeled (HPC: 83.4±0.8%; CTX: 84±2.8% of GABAergic neurons expressed reporter; FIGS. 1C-D). In addition, among the labeled neurons, a clear subset (˜35%) were weakly labeled (FIG. 1E).

Trying to increase the proportion of targeted interneurons, several conserved domains identified within the Dlx5/6 genomic region were tested. The human domain overlapping ml56ii (Ghanem et al., 2003) (h56iiD, h56iiR, FIG. 1A) was inactive in the rodent brain irrespective of orientation (FIG. 9B), and was not characterized further. Consistent with the findings, previous reports using the zebrafish zI46ii domain in transgenic mice indicated inefficient reporter expression in the embryonic forebrain (Zerucha et al., 2000).

In contrast, the h56D promoter, incorporating 836 base pairs of human DNA encompassing and extending beyond the conserved ml56i region (Ghanem et al., 2003), supported reporter expression in nearly all mouse GABAergic interneurons (HPC: 94.9±1.0%; CTX: 92.8±1.4% labeled neurons were GABAergic; FIGS. 1F-G, FIG. 9C). No reporter expression from the h56D promoter was observed in hippocampal excitatory pyramidal neurons.

In the reverse orientation, h56R labeled both excitatory and inhibitory neurons (FIG. 9C), suggesting that these enhancer elements acquire orientation selectivity when positioned near a transcription start site. Moreover, h56D, while in the direct orientation with respect to chromosomal placement, was inverted compared to the sequences used previously in mice and viruses (h/mDlx, FIG. 1A) to target GABAergic interneurons (Dimidschstein et al., 2016; Ghanem et al., 2003; Lee et al., 2014; Potter et al., 2009; Zerucha et al., 2000). The apparent discrepancy may be due to the differences in the origin and span of our enhancer domain compared to those used previously.

The goal was to use these viral constructs in animals where transgenic strains are not available. To this end, it was checked whether or not the h56D promoter could restrict transgene expression to GABAergic neurons of another rodent. Injections into the cortex and hippocampus of the Mongolian gerbil, a popular model for auditory studies, demonstrated that here too GABAergic interneurons were targeted with high specificity (Gerbil HPC: 98.4±1.6%, Gerbil CTX: 83.6±0.4% of targeted neurons were GABAergic; FIGS. 1F-G). In contrast to injections in the forebrain, none of the promoters tested was active in the GABAergic neurons of the inferior colliculus in mouse or gerbil (FIG. 9D), consistent with mesencephalic origin of resident interneurons and the corresponding lack of Dlx gene expression in the midbrain (Bulfone et al., 1993; Lahti et al., 2013). The effectiveness of h56D in mouse and gerbil cortex and hippocampus suggests that it is broadly applicable in rodent models.

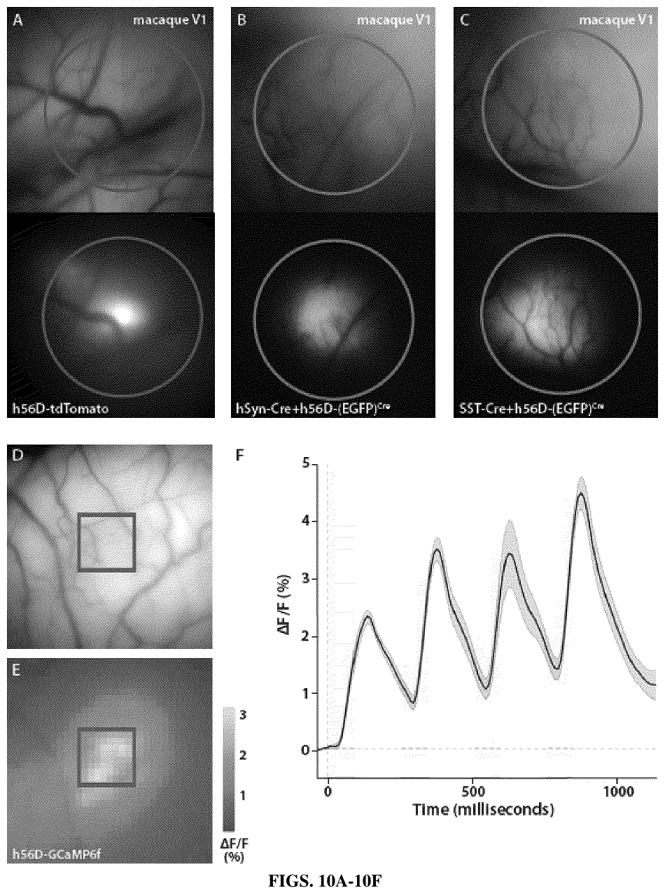

It was next tested the h56D efficacy in the primate by injecting the visual cortex of marmoset and found that the viral vector supported highly specific reporter expression—nearly all labeled neurons were GABAergic (Marmoset CTX: 96.5±1.6%). Reporter expression was detected across all cortical layers in the vicinity of the injection site (88.0±1.4% regional coverage of GABAergic neurons; FIGS. 1F-G). Robust and stable reporter expression was also observed at five sites in the visual cortex of three macaque monkeys. Direct expression from the h56D promoter was seen at four of those sites in two macaques. In addition, at one site macaque reporter was restricted to putative GABAergic interneurons using SYN-Cre and h56D-(EGFP)^{Cre} viruses (FIGS. 10A-B).

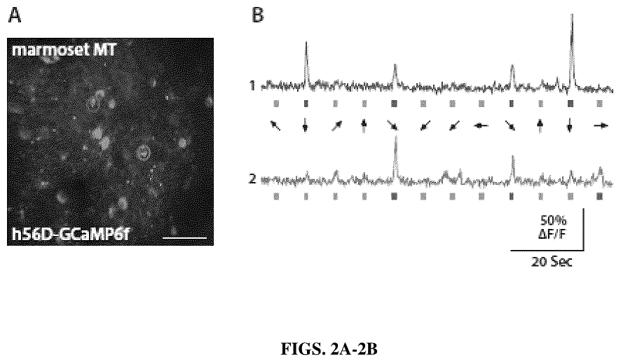

To demonstrate that h56D viral vectors could be used to record functional responses from primate cortical inhibitory neurons, marmoset area MT (FIG. 2A) and rhesus macaque primary visual cortex (V1, FIGS. 10D-E) were injected with viral vectors encoding GCaMP6f. Two-photon imaging of the marmoset cortex revealed differential visually-evoked fluorescence changes in response to distinct motion stimuli (FIG. 2B). Wide-field imaging at 3 injection sites in two macaques likewise uncovered robust fluorescence changes related to the repeated presentations of visual stimuli (FIG. 10E-F). These findings buttress the proposition that conserved gene-regulatory elements can support cross-species cell type-specificity and demonstrate that the h56D promoter can be used to reveal the functional characteristics of primate inhibitory neurons.

### Composition of Targeted GABAergic Neuron Pool:

Next, the complement of GABAergic neurons accessed by h12R and h56D promoters was examined using in situ mRNAs probes for parvalbumin (PV), somatostatin (SST), neuropeptide-Y (NPY) and vasoactive intestinal peptide (VIP), molecular markers for the predominant GABAergic cell populations in the neocortex and hippocampus (Armstrong et al., 2012; Freund and Buzsáki, 1996; Klausberger and Somogyi, 2008; Rudy et al., 2011).

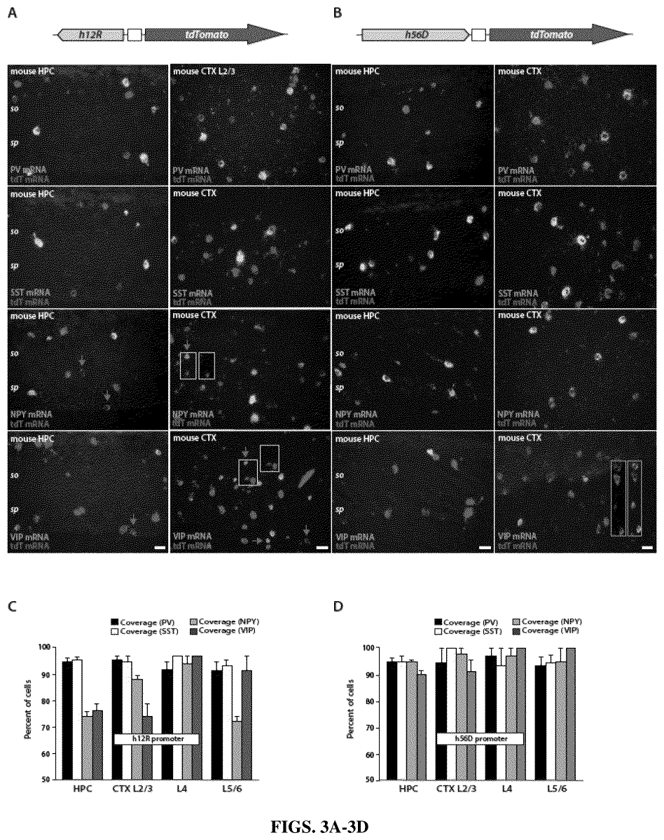

The h12R promoter was active in nearly all mouse PV⁺ and SST⁺ neurons (FIG. 3). The NPY⁺ and VIP⁺ coverage, however, was incomplete: NPY⁺ neurons were underrepresented throughout the dorsal hippocampus (FIG. 3A, C); unlabeled NPY⁺ cells also accounted for approximately 10 percent of the NPY⁺ population in cortical layer 2/3 (90.3±1.7% labeled) and 25 percent in layer 5/6 (73.3±2.0% labeled), while almost all layer 4 NPY⁺ cells were labeled (FIG. 3C). In the hippocampus, excluded VIP⁺ cells were primarily restricted to the pyramidal layer, whereas in the superficial layers of the neocortex (layer 2/3) approximately 25 percent of VIP⁺ cells were not labeled (FIGS. 3A, C). Furthermore, it was observed that, even within the included neuron populations, expression from h12R was not uniform—NPY⁺ cells, for example, segregated into clearly distinguishable groups of high and low expressers. Promoter strength variability was less apparent among PV⁺ and SST⁺ cells, in part due to especially strong in situ signals. Generally, the reporter expression variability may have reflected developmental and functional cell heterogeneity within the targeted GABAergic populations (Gelman et al., 2009; Petilla Interneuron Nomenclature Group et al., 2008; Tricoire and Vitalis, 2012).

In contrast, the h56D promoter supported more uniform reporter expression in each of the PV⁺, SST⁺, NPY⁺ and VIP⁺ GABAergic cell classes (FIGS. 3B, D), consistent with near-comprehensive coverage of GABAergic interneurons (FIG. 1G). In sum two GABAergic interneuron-specific promoters were constructed: h56D, which provides genetic access to all interneuron subclasses, and h12R, which provides access to subsets of interneurons. use these promoters can be used to further refine interneuron targeting using set intersection and set difference strategies.

### Set Intersection Strategy to Target SST⁺ Interneurons:

In rodents, SST⁺ interneurons account for approximately 30 percent of cortical GABAergic cells (Freund and Buzsáki, 1996; Jinno and Kosaka, 2006; Rudy et al., 2011). SST⁺ interneurons primarily innervate dendritic arbors of principal neurons to regulate excitatory input integration and dendritic excitability (Chiu et al., 2013; Lee et al., 2013; Lovett-Barron et al., 2014; 2012; Muñoz et al., 2017; Pfeffer et al., 2013; Royer et al., 2012; Xu et al., 2013). SST⁺ interneurons play key roles in both sensory processing in the neocortex and learning in the hippocampus (Adesnik et al., 2012; Lovett-Barron et al., 2012; 2014). However, tantalizingly little is known about specific roles of SST⁺ neurons in primates, as these cells have been largely inaccessible.

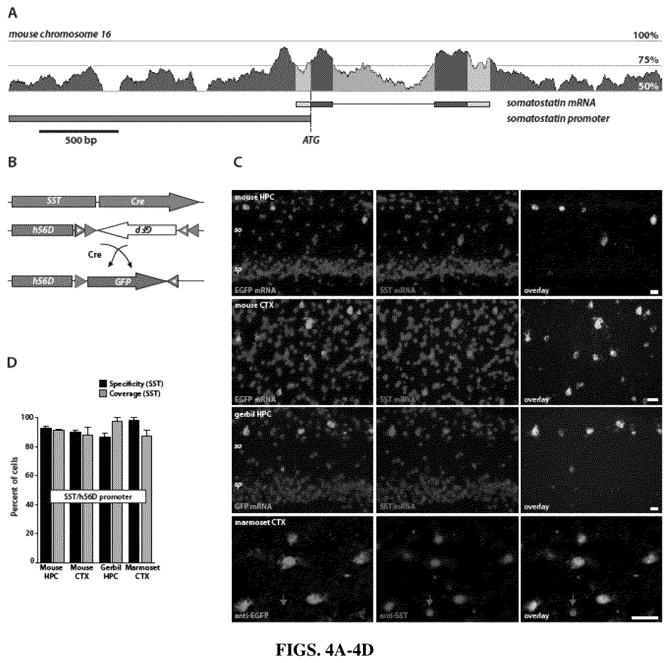

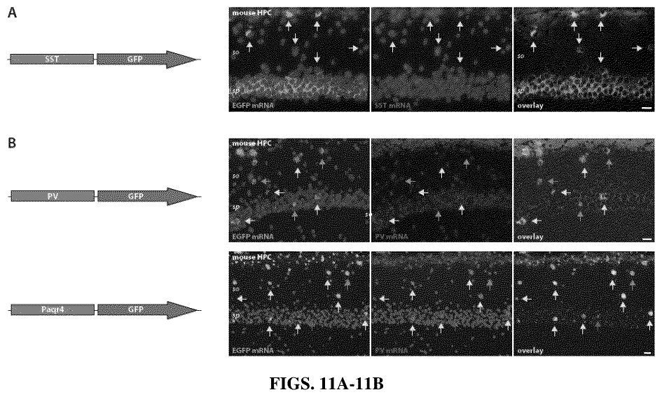

To target SST⁺ neurons, a candidate regulatory domain was identified upstream of the somatostatin gene that was conserved between mouse and human genomes (ECR Browser, Ovcharenko et al., 2004; FIG. 4A). Two rAAV vectors were constructed. The first, SST-EGFP, was fitted with a 2000 base pair putative regulatory domain found just upstream of the mouse somatostatin start codon. When used alone in the mouse hippocampus, EGFP was expressed in SST⁺ GABAergic interneurons, but also in dorsal CA1 excitatory neurons (FIG. 11A). A second vector was then constructed, SST-Cre, and co-injected it with h56D-(EGFP)^{Cre}, intending to restrict fluorophore expression from the h56D GABAergic promoter to neurons expressing the Cre recombinase from the SST promoter (FIG. 4B). Together, this set intersectional approach using two viruses reliably confined reporter expression to GABAergic SST⁺ interneurons in the mouse and gerbil hippocampus and mouse neocortex (Mouse HPC: 92.3±1.5%; Mouse CTX: 90.2±1.5%; Gerbil HPC: 86.7±2.8%, FIG. 4C-D). SST⁺ interneurons at the marmoset cortical layer 2/3 injection site were likewise specifically labeled (Marmoset CTX: 98.5±1.4%; FIG. 4C-D). The two-virus mix also functioned in the macaque cortex, but cell identity has not independently confirmed (FIG. 10C). The SST⁺ neuron targeting strategy also worked when Flp recombinase (Kranz et al., 2010; Raymond and Soriano, 2007) was used in place of Cre (FIG. 12), offering the means to access a second cell population in animals that already express Cre, such as in PV-Cre mice (FIG. 12B).

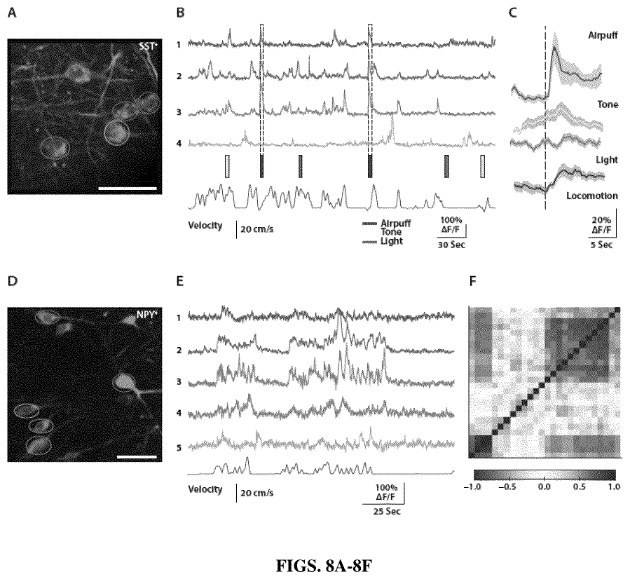

To test whether or not this set intersectional approach could support functional Ca²⁺ imaging of SST⁺ neurons in vivo, rAAV vectors SST-Cre and h56D-(GCaMP6f)^{Cre} were co-injected into dorsal hippocampal area CA1 of wild type mice. A role for SST⁺ neurons was previously demonstrated in responding to aversive cues (Lovett-Barron et al., 2014). Therefore, concurrent with imaging, mice were subjected to pseudorandom discrete stimuli consisting of light flashes, tones and mildly aversive air-puffs to the snout (Lovett-Barron et al., 2014). Indeed, robust GCaMP6f responses were detected in CA1 stratum oriens SST⁺ neurons (FIG. 8A). Air-puffs, but not light flashes or tones, evoked strong responses in most SST⁺ cells (FIG. 8B-C). These observations are consistent with a previous report that had relied on SST-Cre knock-in mice and the SYN-(GCaMP6f)^{Cre} virus (Lovett-Barron et al., 2014), confirming the suitability of the set intersectional cell targeting strategy, and specifically the h56D promoter, which here set the level of GCaMP6f expression, for functional imaging in rodents.

### Set Intersection Strategy to Target PV⁺ Interneurons:

Parvalbumin-expressing (PV⁺) interneurons represent another major inhibitory subclass in the mammalian cortex and hippocampus. PV⁺ basket and axo-axonic cells are key regulators of brain rhythms, and they are intimately involved in the microcircuitry of sensory processing, memory formation and critical period plasticity (Cobb et al., 1995; Klausberger and Somogyi, 2008) Dysfunction of PV⁺ interneurons has been linked to autism and schizophrenia (Lewis et al., 2005).

To identify a promoter that is selectively active in PV⁺ interneurons, a conserved region was first tested upstream of the parvalbumin gene, a tactic that had worked well in the search for the SST promoter. However, the resulting construct showed little PV selectivity in the mouse brain (FIG. 11B).

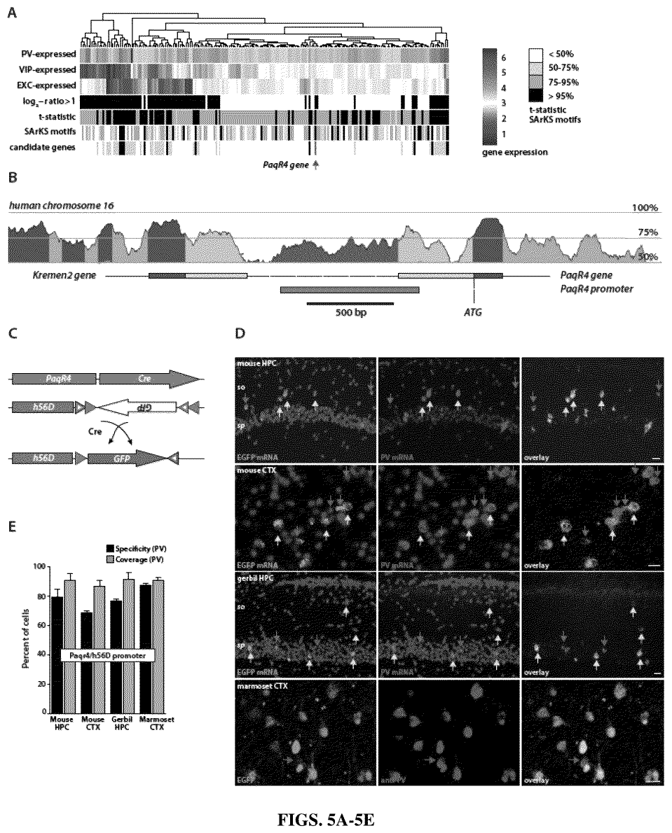

A general and rational approach was then developed for promoter candidate selection that aimed to minimize the hit-or-miss aspect of existing strategies. The goal was to build a computational tool, SArKS (Wylie et al., 2018), that mines the growing body of RNAseq data for sequence motifs associated with cell type-specific gene expression. The algorithm then uses a regression model to rank genes whose promoter regions contain those motifs (FIG. 5A).

SArKS was used to analyze one of the first datasets that compared the transcriptome of PV⁺ neurons to that of other non-overlapping cell subclasses (Mo et al., 2015). Importantly, Mo, et al., also generated epigenetic maps for their cell subclasses. The top 11 genes met the following criteria: (1) their expression was above a set threshold in PV⁺ neurons, but below that threshold in other neuron subclasses; (2) their chromatin was accessible in all cell subclasses; (3) their log 2-ratio of expression in PV⁺ neurons to other neuron subclasses had to exceed 1 (i.e. a minimum 2-fold increase in average expression level); (4) they ranked in the top 5% by t-statistic comparing expression level in PV⁺ neurons to levels in other neuron subclasses; and (5) they ranked in the top 5% by the SArKS motif regression model (FIG. 5A). Importantly, the SArKS regression model excluded a substantial number of PV⁺ neuron-enriched genes. The parvalbumin gene itself fulfilled most of these requirements, but its chromatin was differentially accessible (Mo et al., 2015); the PV promoter was consequently eliminated from contention.

Among the genes highlighted by SArKS was PaqR4, a member of the progestin receptor family (Tang et al., 2005). PaqR4 transcript was more abundant in PV⁺ neurons compared to VIP⁺ neurons but was not among the most abundant transcripts (FIG. 5A). Its expression pattern in the mouse brain—among the pyramidal cells in hippocampal region CA1 and in central cortical layers—is generally similar to that of PV (Allen Brain Atlas, Lein et al., 2007). Its putative regulatory region is fairly short, ˜1 kb, and mostly conserved between mouse and human (FIG. 5B). When tested alone in the mouse hippocampus, rAAV encoding the human PaqR4 promoter labeled most PV⁺ neurons, but also some excitatory and putative glial cells (FIG. 11B). However, an intersectional approach using h56D to refine labeling (FIG. 5C), as described above to target SST⁺ neurons, yielded a highly enriched population of PV⁺ cells in rodent cortex and hippocampus (Mouse HPC: 79.8±4.9%, Mouse CTX: 69.1±1.4%, Gerbil HPC: 76.8±1.3%; FIG. 5D-E).

To test whether or not these constructs could support reporter expression in PV⁺ neurons of the primate neocortex, the marmoset cortical area MT was injected and labeled neurons were examined post-mortem using anti-PV immunostaining (FIG. 5D). Nearly 90% of PV⁺ neurons in the vicinity of the cortical layer 4 injection site expressed the reporter meanwhile, 87% of virus-labeled neurons were PV⁺, a higher percentage than was seen in rodents (Marmoset CTX: 87.4±1.4% FIGS. 5D-E).

### Set Difference Strategy to Target Excitatory Neurons:

The targeting of excitatory neurons with viruses is generally achieved using a section of the mouse calcium/calmodulin-dependent protein kinase II alpha (CaMKIIα) promoter (Dittgen et al., 2004). However, under certain conditions this promoter may also be active in inhibitory interneurons (Nathanson et al., 2009a; Schoenenberger et al., 2016) and inactive in subsets of cortical excitatory neurons (Huang et al., 2014; Wang et al., 2013; Watakabe et al., 2015). Moreover, there is considerable regional variation in the expression of endogenous CaMKIIα in the rodent and primate brains (Benson et al., 1992; 1991).

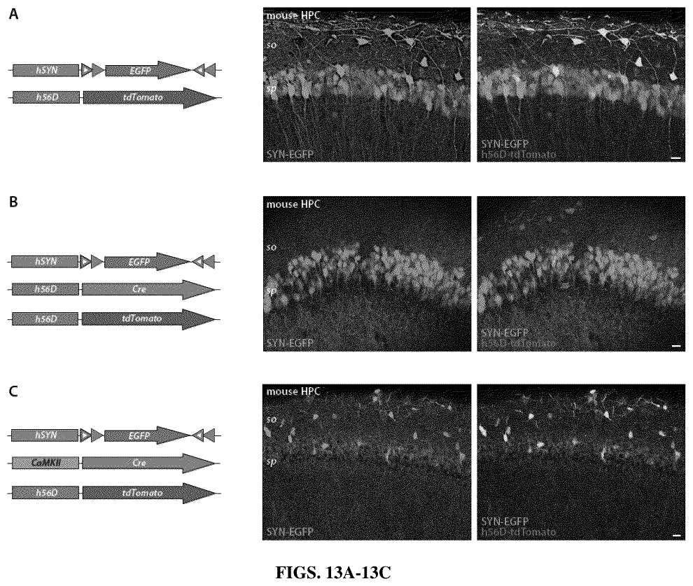

Relying on the broad interneuron specificity of the h56D promoter, a two-virus strategy was tested for accessing excitatory-only neurons by effectively subtracting the inhibitory interneuron population from all neurons (FIGS. 13A-B). The set difference strategy is unlike the set intersection approach in that the vectors are not fully interdependent: the primary vector is active until expression is blocked; an inefficient block results in false positives.

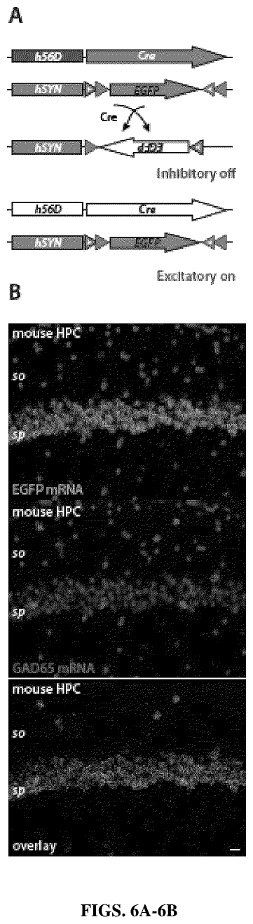

One viral vector was constructed where a floxed reporter protein in the forward (sense) orientation was transcribed from a pan-neuronal human synapsin promoter (SYN-(EGFP_{FWD})^{Cre}) (Borghuis et al., 2011b; Schoch et al., 1996). A second vector expressed the Cre recombinase from the h56D inhibitory promoter (h56D-Cre, FIG. 6A). When co-injected into the mouse dorsal hippocampus, the virus-encoded recombinase converted the sense reporter orientation to an antisense orientation only in inhibitory interneurons, and thus restricted reporter expression to excitatory neurons without relying on the CaMKIIα promoter (11.2±1.0% GAD65⁺ cells remained labeled, consistent with neuron coverage when using h56D promoter; FIG. 6B). If GABAergic interneurons account for approximately 10 percent of mouse hippocampal neurons, a false-positive rate was estimate for the set difference strategy (i.e. that an excitatory cell turns out to be inhibitory) of 1-2 percent.

### Set Difference Strategy to Target Subsets of Neuropeptide-Y Interneurons:

A set difference strategy could also be used to access subsets of NPY⁺ interneurons. These are a diverse population in rodents, both with respect to their origin (Fuentealba et al., 2008; Gelman et al., 2009; Miyoshi and Fishell, 2011; Tricoire and Vitalis, 2012) and function. In addition to modulating individual excitatory neuron firing rates through feed-forward inhibition, NPY⁺ interneurons form gap junctions with each other and nearby GABAergic cells, potentially coupling cortical networks (Armstrong et al., 2012; Fuentealba et al., 2008; Simon et al., 2005). As a neuropeptide, NPY can also promote neurogenesis (Decressac et al., 2011) and acts as an anti-epileptic (Baraban et al., 1997; Noé et al., 2008). Since NPY⁺ interneurons had previously only been examined using transgenic mice (Milstein et al., 2015; van den Pol et al., 2009), it was decided to try targeting them using our new GABAergic promoters.

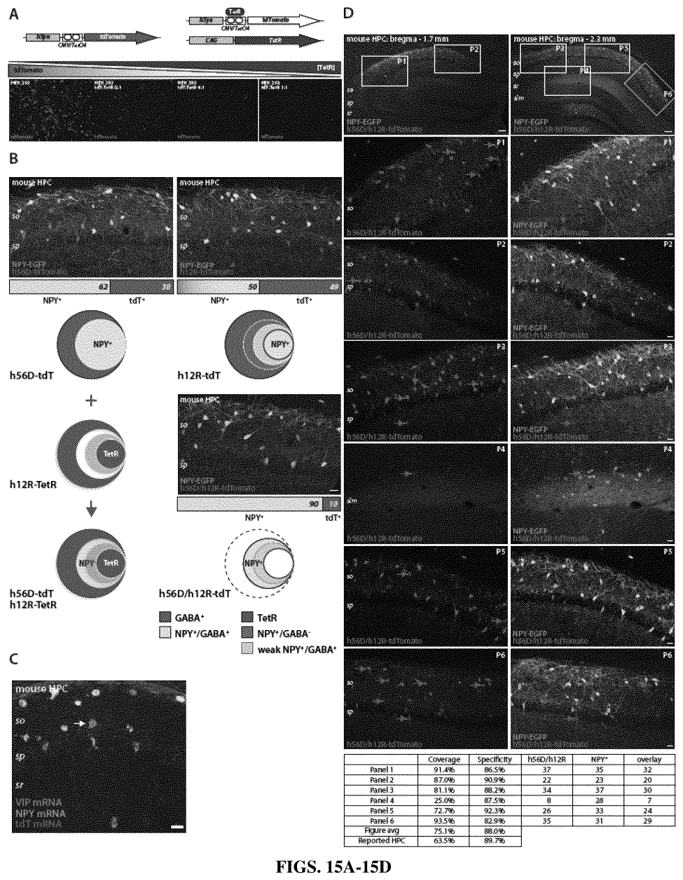

It was noted that the h12R promoter labeled approximately 85% of GABAergic neurons in the mouse cortex and hippocampus (FIG. 1C-D) and that many of the excluded cells were NPY⁺ and VIP⁺ (FIGS. 3A, C). Moreover, nearly half of NPY⁺ neurons targeted by h12R (39.1±5.5%) expressed the reporter weakly (FIG. 15B). Thus, h12R promoter demonstrated little to no activity in a significant fraction of NPY⁺ neurons. Based on these observations, combinatorial methods were tested for targeting subsets of NPY⁺ interneurons, which have heretofore been inaccessible using transgenics or viral approaches.

Differential promoter activity, such as that seen for h12R, is consistent with recent reports that gene expression variations across cortical and hippocampal interneuron subclasses represent distinctions in degree rather than distinctions in kind (Földy et al., 2016; Harris et al., 2017; Mo et al., 2015; Paul et al., 2017; Tasic et al., 2016; Zeisel et al., 2015).

Gradations in gene expression have likewise been detected within relatively homogeneous cell classes, such as among hippocampal excitatory neurons (Cembrowski et al., 2016; Thompson et al., 2008, but see Lein et al., 2007). These studies support the notion that transcriptome variations underlie functional heterogeneity within traditional neuron classes, but also challenge efforts to group and target specific neurons based on single distinguishing genetic markers.

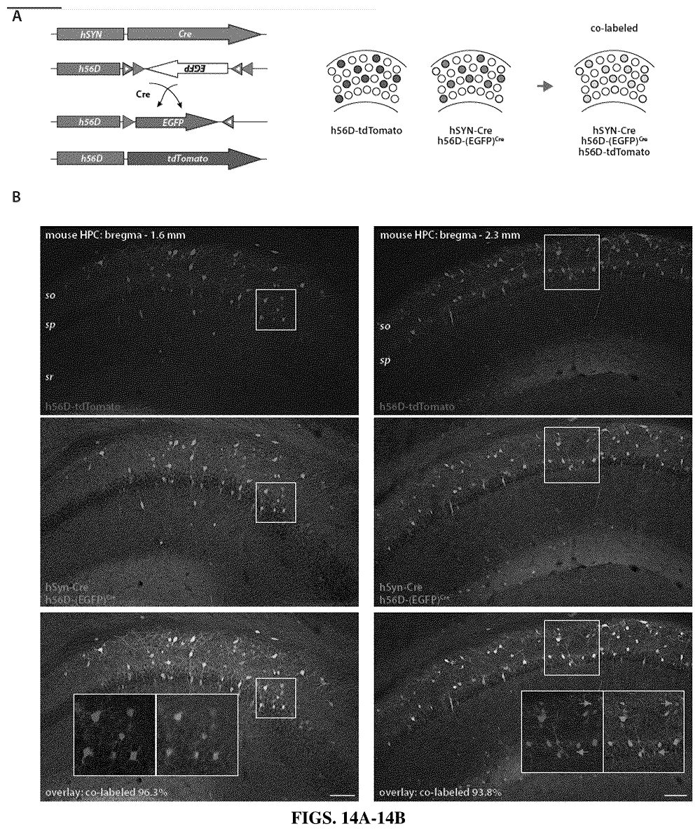

To examine if differences in h12R versus h56D promoter activity could be harnessed to access functionally distinct subsets of NPY⁺ interneurons, pairs of interdependent viruses were built for tunable cell type-specific heterologous protein expression. One vector from each pair contained a tetracycline regulon—a dimerized tetracycline operator (TetO4) inserted into the cytomegalovirus minimal promoter (Yao et al., 1998). A tetracycline repressor (TetR, Beck et al., 1982; Hillen and Berens, 1994) was encoded by the second vector (FIG. 15A). This scheme is different from the better known Tet_{ON/OFF} Systems (Gossen and Bujard, 1992; Gossen et al., 1995), where the repressor acts as a transcription factor, in that here the repressor can block read-through from any TATA box-containing promoter, preserving cell type-specific expression for both reporter and repressor. Moreover, unlike Cre recombinase-dependent schemes, which employ an enzyme and are therefore more difficult to regulate, TetR blocks transcription stoichiometrically, a useful property for exploiting promoter strength variations. Indeed, when the TetR system was tested in cultured fibroblasts transfected with different ratios of reporter and repressor constructs, TetR blocked reporter expression in a dose-dependent fashion (FIG. 14A).

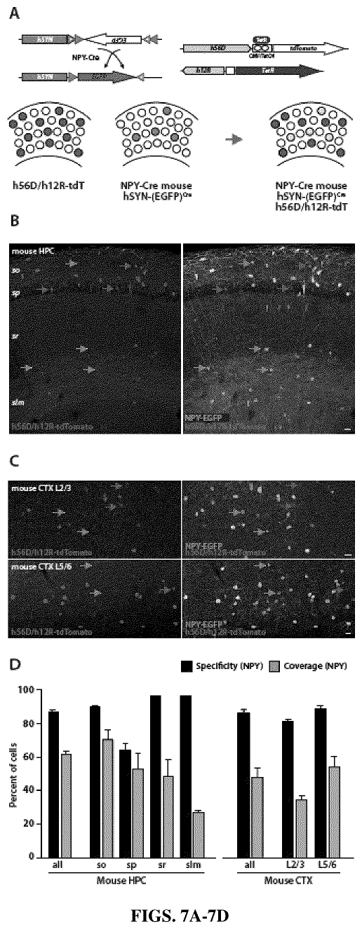

To characterize NPY⁺ neurons where the h12R and h56D promoters are differentially active, mixes of h56D_{TetO4}-tdTomato, h12R-TetR and hSYN-(EGFP)^{Cre} vectors were injected into brains of knock-in NPY-Cre mice (Milstein et al., 2015). The hSYN-(EGFP)^{Cre} labeled the endogenous NPY⁺ neurons green, while the inhibitory viruses additionally labeled a subset of neurons red (FIG. 7A). TetR blocked reporter expression in neurons where the h12R and h56D promoters were comparably active (most EGFP⁻/tdT⁺ GABAergic interneurons), but not in inhibitory cells where h12R promoter was weakly active or inactive (EGFP⁺ neurons, FIG. 15B), such that approximately 90 percent of hippocampal and 88 percent of cortical interneurons labeled by the interdependent viruses were NPY⁺ (EGFP⁺/tdT⁺, FIGS. 7B-D, 15B). In the hippocampus, most of the virus-labeled NPY⁻ neurons were VIP⁺, as predicted based on the pattern of h12R expression in VIP⁺ cells (FIG. 3A, C). However, only 63 percent of all hippocampal and 45 percent of all cortical NPY⁺ cells were labeled (FIG. 7B-D). In line with the h12R expression pattern (FIG. 1C), labeling was stratified: in the mouse hippocampus, virus-labeled NPY⁺ neurons were abundant in stratum oriens, but largely absent in strata radiatum and lacunosum-moleculare (FIG. 7B, 15E). In addition, cortical layer 2/3 had fewer labeled neurons than layer 5/6 (FIG. 7C-D).

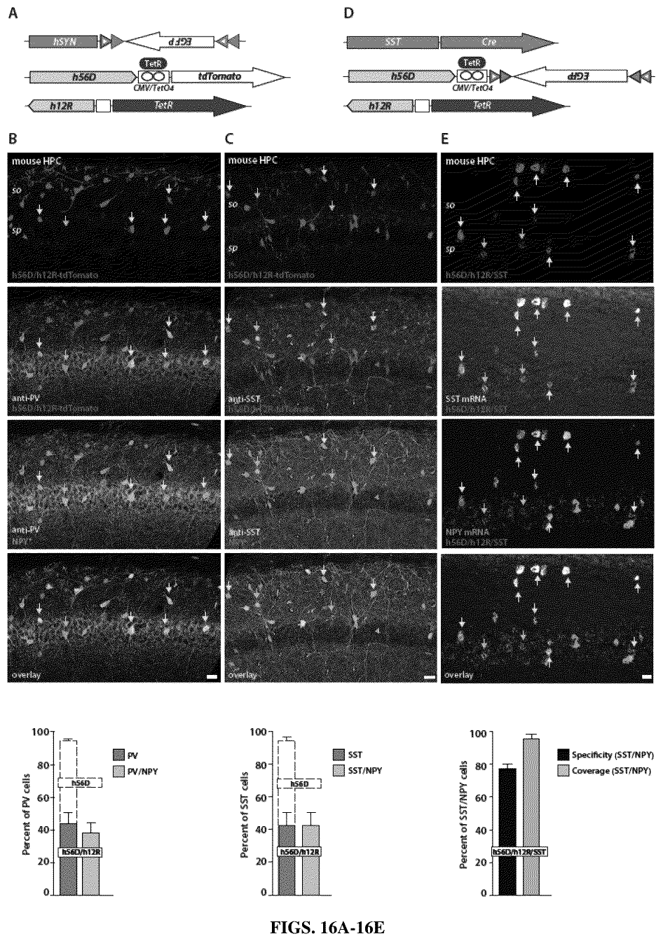

The characteristics of the virus-labeled cells were examined and two subclasses of NPY⁺ interneurons were uncovered. Immunostaining for PV showed that, compared to h56D alone, approximately half of all PV⁺ neurons had been labeled by the interdependent viruses, the majority in stratum pyramidale (44.1±6.7%, FIG. 16B). The labeled PV⁺ neurons were predominantly NPY⁺ (86.8% of labeled PV⁺ neurons were PV⁺/NPY⁺), while the unlabeled PV⁺ neurons were NPY⁻ (FIG. 16B). Therefore, the NPY⁺/PV⁺ subclass specificity was high and the NPY⁺/PV⁺ coverage was nearly comprehensive (95±8.2% of NPY⁺/PV⁺ neurons had been labeled by the viruses).

Immunostaining also revealed that the h56D/h12R interdependent viruses labeled less than half of hippocampal SST⁺ neurons (FIG. 16C). However, this entire population comprised SST⁺/NPY⁺ neurons in stratum oriens (FIG. 16C), of which 80 percent (80.2±8.2% coverage) had been labeled, providing a way to selectively enrich for this subset of interneurons (Jinno and Kosaka, 2004). This population was distinct from the PV⁺/NPY⁺ neurons described above, consistent with the reported segregation of neocortical PV⁺ and SST⁺ interneuron subclasses (Rudy et al., 2011).

To demonstrate that the SST⁺/NPY⁺ interneuron subpopulation could be specifically targeted, which cannot easily be accessed using transgenic animals, a double restriction was set up in wild type mice. rAAVs SST-Cre, h56D_{TetO4}-(EGFP)^{Cre} and h12R-TetR were co-injected, imposing the SST requirement onto the subset of NPY⁺ neurons (FIG. 16D). With this cocktail, the SST⁺/NPY⁺ neurons were reliably isolated in the mouse hippocampus (95.6±2.8% of SST⁺/NPY⁺ neurons had been labeled, FIG. 16E).

Prior to generating an NPY-Cre mouse line (Milstein et al., 2015), it had been difficult to study these neurons in isolation. Without a template for NPY cell activity during a behavioral task, the study settled for a confirmation that subtractive expression of GCaMP6f using the two-virus system supported functional imaging in vivo. Viral vectors h56DTetO4-GCaMP6f and h12R-TetR were co-injected into dorsal hippocampi of wild type mice and a preliminary in vivo head-fixed two-photon Ca²⁺ imaging was conducted during head-fixed running on a cue-rich treadmill. Stratum oriens, but not stratum pyramidale, neurons expressed abundant reporter (FIG. 8D). Labeled cells exhibited reliable locomotion-related activity, with subset displaying tight cross-correlation in the activity profiles (FIGS. 8E, F).

The set difference method for cell type-specific expression regulation represents a proof-of-concept for a new transgenics-independent way to target defined classes of neurons in the brain. While a fixed molar ratio of reporter and repressor vectors was used to enrich for NPY neurons, different promoters and ratios could access other cell subsets within and across traditional neuron classes for imaging and manipulation. Importantly, unlike recombinase-dependent techniques for expressing foreign proteins, the TetR-dependent approach is selective, tunable and reversible when regulated using injectable doxycycline or doxycycline added to animal chow. In addition, the TetR set difference technique can be used orthogonally with recombinanses to target two cell classes, or jointly with recombinases, as demonstrated for SST⁺/NPY⁺ neurons above, to examine previously inaccessible neuronal circuit elements.

These multi-virus techniques for accessing key subsets of neurons represent viable alternatives to single cell type-specific promoters and provide ample protein expression for nuanced functional studies, including in vivo imaging and manipulation studies in the primate, of the diverse cell populations that comprise the cortex and hippocampus. Indeed, bringing methods that have enabled breakthrough examinations of rodent neural circuit mechanisms to the primate has been a priority for our laboratories. The present techniques can also be combined to further refine cell targeting or used orthogonally in circuit-level experiments. These general methods offer a timely blueprint applicable to many neuron classes and species that will aid the transgenics-independent brain-wide interrogations of functionally significant cell populations.

### Conservation of Non-Coding DNA:

SArKS examines differences in gene expression across cell classes based on cell-specific transcriptome data. Such data have now been collected from genetically-defined cell classes in rodents (Hodge et al., 2019; Mo et al., 2015), but not from primates. Indeed, this chicken-and-egg problem-needing cell-specific transcriptome data to be able to define and access cell classes-represents a significant hurdle in engineering vectors for NHP research. Fortunately, comparisons of distantly-related vertebrate genomes have demonstrated that conserved non-coding DNA, especially in the vicinity of developmentally-important genes, can support shared regulatory regimes (Woolfe et al., 2005; Hardison et al., 1997; and Elgar, 1996).

To circumvent the lack of primate cell-specific data, SArKS was used to identify candidate mouse regulatory domains and have then examined these domains for elevated rodent-primate sequence conservation. This strategy is supported by the promiscuity of transcription factors, which are known to tolerate subtle sequence variations (Gumucio et al., 1996; Letovsky and Dynan, 1989) and has helped uncover human regulatory regions for accessing GABAergic and parvalbumin-expressing forebrain neurons in both rodent and primate. The inventors anticipate that the presence of cross-species sequence conservation within putative promoters will continue to be an important parameter when engineering viral vectors that are active in multiple species. One practical benefit of such conservation is that many candidate promoters can be pre-screened in mouse.

### Chromatin Accessibility:

One important parameter that was considered when selecting differentially expressed genes for SArKS analysis is whether or not the chromatin is accessible in the vicinity of differentially expressed genes, where cell-specific transcription factors must bind. From an experimental perspective, genomic DNA may appear inaccessible because it is epigenetically modified, blocking transcription factor binding; alternatively, a bound transcription factor can render chromatin inaccessible while enabling transcription. The inventor filtered promoter regions that are not accessible in every cell population that was compared because it was desired to harness differential gene expression mechanisms supported entirely by cell-specific transcription factors (Davidson, 2010). Variable gene expression where the binding of a ubiquitous transcription factor is epigenetically regulated is at odds with our sequence-based strategy and cannot be reproduced when using viral vectors whose genomes are not similarly modified. However, a screen for inaccessible chromatin in the cells of interest may be a useful strategy when examining the effects of distal sequences, such as enhancers, on gene expression (Bell et al., 2011). There, differential accessibility may indeed result from cell-specific transcription factor binding (Li et al., 1999), which can foster cell-specific expression (Hrvatin et al., 2019; Graybuck et al., 2019).

As described above, the h12R promoter was active in nearly all mouse PV⁺ and SST⁺ neurons (FIG. 3). However, the NPY⁺ and VIP⁺ coverage was incomplete: NPY⁺ neurons were underrepresented throughout the dorsal hippocampus (FIG. 3A, C); cortical layers 2/3 and 5/6 also contained unlabeled NPY⁺ cells (coverage: 1 2/3 90.3±1.7%; 1 5/6 73.3±2.0%), and almost all layer 4 NPY⁺ cells were unlabeled (FIG. 3C). In the hippocampus, excluded VIP⁺ cells were primarily restricted to the pyramidal layer, whereas in the superficial layers of the neocortex (layer 2/3) approximately 25 percent of VIP⁺ cells were not labeled (FIG. 3A, C). Furthermore, it was observed that, even within the included neuron populations, expression from h12R was not uniform: unlike PV⁺ and SST⁺ cells, NPY⁺ neurons segregated into clearly distinguishable groups of high and low expressers, perhaps consistent with developmental and functional cell heterogeneity within these GABAergic populations (Gelman et al., 2009; Petilla Interneuron Nomenclature Group et al., 2008; Tricoire and Vitalis, 2012).

In contrast, the h56D promoter supported uniform reporter expression in each of the PV⁺, SST⁺, NPY⁺ and VIP⁺ GABAergic cell classes (FIG. 3B, D). In sum, two GABAergic interneuron-specific promoters were constructed: h56D, which provided genetic access to all interneuron subclasses, and h12R, which provided access to subsets of interneurons. We could now use these promoters to further refine interneuron targeting with set intersection and set difference strategies

To identify a promoter that is selectively active in PV⁺ interneurons, the inventor first tested a conserved region upstream of the parvalbumin gene, a tactic that had worked well in the search for the SST promoter. However, the resulting construct showed little PV selectivity in the mouse brain (FIG. 11B).

The recently developed algorithm, SArKS (Wylie et al., 2018), and mined RNAseq data (Mo et al., 2015) was then used for sequence motifs associated with cell type-specific expression in PV⁺ neurons. Among the genes highlighted by SArKS was PaqR4, a member of the progestin receptor family (Tang et al., 2005). When tested alone in the mouse hippocampus, rAAV encoding the human PaqR4 promoter labeled PV⁺ neurons, but also some excitatory and putative glial cells (FIG. 11B). However, an intersectional approach using h56D to refine labeling (FIG. 5C), as described above to target SST⁺ neurons, displayed high specificity for PV⁺ cells in rodent cortex and hippocampus (Mouse HPC: 79.8±4.9%; Mouse CTX: 69.1±1.4%; Gerbil HPC: 76.8±1.3%; FIG. 5D-E).

To identify candidate promoters for accessing PV⁺ interneurons, the inventor re-analyzed a mouse RNAseq data set (Mo et al., 2015), where Cre recombinase-expressing mice were bred with a Cre-dependent fluorescent reporter mouse strain (Ai14; Madisen et al., 2010) to tag and isolate neocortical excitatory neurons, PV⁺ neurons and VIP⁺ neurons. First, Kallisto (Bray et al., 2016) was used to localize transcription start sites (TSSs) for the expressed genes. Kallisto reported 73,912 distinct transcripts detected with nonzero estimated count in at least one of the analyzed samples. After filtering out transcripts that had low estimated counts or low average or low variance in transcripts-per-million (TPM) normalized expression levels, 29,164 distinct transcripts remained; these transcripts represented 11,857 distinct genes. Only a single transcript variant having the highest average TPM for each gene was retained. For each of the remaining transcripts, we checked whether or not the TSS was located within a chromatin-accessible region in each of the neuron classes (as measured by ATACseq; Mo et al., 2015). In order to focus on those genes for which expression variability between neuron classes is most likely to be a function of promoter sequence as opposed to chromatin state, the inventor eliminated all genes where the TSS was not contained within a chromatin-accessible region in every neuron class. The parvalbumin gene itself fulfilled most of the enumerated criteria, but its chromatin was differentially accessible (Mo et al., 2015); the PV promoter was consequently eliminated from contention. The upstream regions (˜3 kb) of the remaining 6,326 genes were examined using SArKS (Wylie et al., 2018) to find motifs (k-mers) whose occurrence in a set of promoter sequences correlated with an input metric of differential expression: a t-statistic comparing the TPM-normalized RNA transcript abundance in PV⁺ neurons versus PV⁻ neurons. SArKS first identified motifs by employing smoothing over subsequences by sequence similarity and then identified multi-motif domains (MMDs) by additionally smoothing over spatial proximity, using a permutation testing approach to establish statistical significance. The counts of how many times each uncovered motif occurred in a promoter region was then used as the feature vector for training a regression model to predict differential expression, again quantified as a t-statistic. The predicted scores from this regression model were then used to rank promoters by SArKS motif content, yielding 11 putative regulatory domains for experimental testing, one of which was for PaqR4 a member of the progestin receptor family (Tang et al., 2005). PaqR4 transcript was more abundant in PV⁺ neurons compared to VIP⁺ neurons but was not among the most abundant transcripts (FIG. 5A), Its expression pattern in the mouse forebrain is similar to that of PV (Allen Brain Atlas, Lein et al., 2007). Its putative regulatory region is fairly short., 1 kb, and mostly conserved between mouse and human (FIG. 5B). When tested alone in the mouse hippocampus, rAAV encoding the human PaqR4 promoter labeled PV* neurons, but also some excitatory and putative glial cells (FIG. 11B). However, an intersectional approach using h56D to refine labeling (FIG. 5C), as described above to target SST⁺ neurons, yielded a highly enriched population of PV⁺ cells in rodent and primate forebrain (FIG. 5),

Reporter expression was also highly specific in PV⁺ neurons of the marmoset cortical area MT (specificity: 87.4±1.4%, coverage: 87.1±3.5%; FIG. 5D-E), higher percentages than were observed in rodent forebrain.

PV+ neurons comprise both basket and chandelier cells. The PaqR4 promoter, which currently targets both neuron subclasses, was altered by deleting each of the four multi-motif domains (MMDs). An initial evaluation indicates that the mix of targeted cells is affected by the combination of MMDs: for example, deletion of the PaqR4 MMD3 reduces the number of SST neurons and increases the number of PV neurons where this engineered promoter is active. Another possibility is to use layer-specific promoters from FIG. 18 that display partial PV specificity. These promoters can be used intersectionally (as described below) with Paqr4 to restrict PV neuron targeting.

Since NPY⁺ interneurons had previously only been examined using transgenic mice (Milstein et al., 2015; van den Pol et al., 2009), the inventor decided to try targeting them using our GABAergic promoters. The h12R promoter demonstrated little to no activity in a significant fraction of NPY⁺ neurons (FIG. 3A, C; Fig S7B). To examine if differences in h12R versus h56D promoter activity could be harnessed to access functionally distinct subsets of NPY⁺ interneurons, pairs of interdependent viruses for tunable cell type-specific heterologous protein expression were built. One vector from each pair contained a tetracycline regulon (TetO4) inserted into the cytomegalovirus minimal promoter (Yao et al., 1998). A tetracycline repressor (TetR, Beck et al., 1982; Hillen and Berens, 1994) was encoded by the second vector (FIG. 15A).

When cultured fibroblasts were transfected with different ratios of such constructs, TetR blocked reporter expression in a dose-dependent fashion (FIG. 14A).

In developing a strategy to target NPY⁺ interneurons, the inventor had noted that the h12R promoter labeled approximately 85% of GABAergic neurons in the mouse cortex and hippocampus (FIG. 1C-D) and that many of the excluded cells were NPY⁺ and VIP⁺ (FIG. 3A, C). Moreover, nearly half of NPY⁺ neurons targeted by h12R (39.1±5.5%) expressed the reporter weakly (FIG. 15B). Thus, h12R promoter demonstrated little to no activity in a significant fraction of NPY⁺ neurons. Based on these observations, combinatorial methods for targeting subsets of NPY⁺ interneurons, which have heretofore been inaccessible using transgenics or viral approaches were tested.

Differential promoter activity, such as that seen for h12R, is consistent with recent reports that gene expression variations across cortical and hippocampal interneuron subclasses represent distinctions in degree rather than distinctions in kind (Földy et al., 2016; Harris et al., 2017; Mo et al., 2015; Paul et al., 2017; Tasic et al., 2016; Zeisel et al., 2015). Gradations in gene expression have likewise been detected within relatively homogeneous cell classes, such as among hippocampal excitatory neurons (Cembrowski et al., 2016; Thompson et al., 2008, but see Lein et al., 2007). These studies support the notion that transcriptome variations underlie functional heterogeneity within traditional neuron classes, but also challenge efforts to group and target specific neurons based on single distinguishing genetic markers.

To examine if differences in h12R versus h56D promoter activity could be harnessed to access functionally distinct subsets of NPY⁺ interneurons, pairs of interdependent viruses for tunable cell type-specific heterologous protein expression were built. One vector from each pair contained a tetracycline regulon—a dimerized tetracycline operator (TetO4) inserted into the cytomegalovirus minimal promoter (Yao et al., 1998). A tetracycline repressor (TetR, Beck et al., 1982; Hillen and Berens, 1994) was encoded by the second vector (FIG. 15A). This scheme is different from the better known Tet_{ON/OFF} Systems (Gossen and Bujard, 1992; Gossen et al., 1995), where the repressor acts as a transcription factor, in that here the repressor can block read-through from any TATA box-containing promoter, preserving cell type-specific expression for both reporter and repressor. Moreover, unlike Cre recombinase-dependent schemes, which employ an enzyme and are therefore more difficult to regulate, TetR blocks transcription stoichiometrically, a useful property for exploiting promoter strength variations. Indeed, when the TetR system was tested in cultured fibroblasts transfected with different ratios of reporter and repressor constructs, TetR blocked reporter expression in a dose-dependent fashion (FIG. 14A).

To characterize NPY⁺ neurons where the h12R and h56D promoters are differentially active, mixes of h56D_{TetO4}-tdTomato, h12R-TetR and hSYN-(EGFP)^{Cre} vectors were injected into brains of knock-in NPY-Cre mice (Milstein et al., 2015). The hSYN-(EGFP)^{Cre} labeled the endogenous NPY⁺ neurons green, while the inhibitory viruses additionally labeled a subset of neurons red (FIG. 7A). TetR blocked reporter expression in neurons where the h12R and h56D promoters were comparably active (most EGFP⁻/tdT⁺ GABAergic interneurons), but not in inhibitory cells where h12R promoter was weakly active or inactive (EGFP⁺ neurons, FIG. 15B), such that approximately 90 percent of hippocampal and 88 percent of cortical interneurons labeled by the interdependent viruses were NPY⁺ (EGFP⁺/tdT⁺, FIG. 7B-D, S7B).

Mixes of h12R and h56D repressor and reporter vectors (respectively) injected into mouse brains labeled high percentages of forebrain NPY⁺ neurons (specificity: HPC 89.7±1.3%; CTX 87.9±1.8%). However, in line with the h12R expression pattern (FIG. 1C), coverage was incomplete and stratified: NPY⁺ neurons were abundant in stratum oriens, but largely absent in hippocampal strata radiatum and lacunosum-moleculare (72.6±6.2% versus 27.8±1.6% coverage; FIG. 7B, S7E); in addition, cortical layer 2/3 had fewer labeled neurons than layer 5/6 (55.6±6.4% versus 35.4±2.3% coverage; FIG. 7C-D). In the hippocampus, the few virus-labeled NPY⁻ neurons were VIP⁺ (FIG. 15C).

Mixes of h12R and h56D repressor and reporter vectors (respectively) injected into mouse brains labeled high percentages of forebrain NPY⁺ neurons (specificity: HPC 89.7±1.3%; CTX 87.9±1.8%). However, in line with the h12R expression pattern (FIG. 1C), coverage was incomplete and stratified: NPY⁺ neurons were abundant in stratum oriens, but largely absent in hippocampal strata radiatum and lacunosum-moleculare (72.6±6.2% versus 27.8±1.6% coverage; FIG. 7B, S7E); in addition, cortical layer 2/3 had fewer labeled neurons than layer 5/6 (55.6±6.4% versus 35.4±2.3% coverage; FIG. 7C-D). In the hippocampus, the few virus-labeled NPY⁻ neurons were VIP⁺ (FIG. 15C).

The inventor proceeded to examine the characteristics of the virus-labeled cells and uncovered two subclasses of NPY⁺ interneurons. Immunostaining for PV showed that, compared to h56D alone, approximately half of all PV⁺ neurons had been labeled by the interdependent viruses, the majority in stratum pyramidale (PV⁺ coverage: 44.1±6.7%; FIG. 16B). The labeled PV⁺ neurons were predominantly NPY⁺ (86.8% of labeled PV⁺ neurons were PV⁺/NPY⁺), while the unlabeled PV⁺ neurons were NPY⁻ (FIG. 16B). Therefore, the NPY⁺/PV⁺ subclass specificity was high and the NPY⁺/PV⁺ coverage was nearly comprehensive (95±8.2% of NPY⁺/PV⁺ neurons had been labeled by the viruses).

Immunostaining also revealed that the h56D/h12R interdependent viruses labeled less than half of hippocampal SST⁺ neurons (SST⁺ neuron coverage: 42.6±7.9%; FIG. 16C). However, this entire population comprised SST⁺/NPY⁺ neurons in stratum oriens (FIG. 16C), of which 80 percent (80.2±8.2% coverage) had been labeled, providing a way to selectively enrich for this subset of interneurons (Jinno and Kosaka, 2004). This population was distinct from the PV⁺/NPY⁺ neurons described above, consistent with the reported segregation of neocortical PV⁺ and SST⁺ interneuron subclasses (Rudy et al., 2011).

To demonstrate that one could specifically target the SST⁺/NPY⁺ interneuron subpopulation, which cannot easily be accessed using transgenic animals, the inventor set up a double restriction in wild type mice. We co-injected rAAVs SST-Cre, h56DTetO4-(EGFP)^{Cre} and h12R-TetR, imposing the SST requirement onto the subset of NPY⁺ neurons (FIG. 16D). With this cocktail, the inventor was able to reliably isolate the SST⁺/NPY⁺ neurons in the mouse hippocampus (95.6±2.8% of SST⁺/NPY⁺ neurons had been labeled, FIG. 16E).

The inventor tested the ability to examine hippocampal NPY⁺ neuron function in vivo. Without a template for NPY⁺ cell activity during a behavioral task, the inventor settled for a confirmation that subtractive expression of GCaMP6f using the two-virus system supported functional imaging. Stratum oriens, but not stratum pyramidale, neurons expressed abundant GCaMP6f (FIG. 8D) and, based on preliminary in vivo head-fixed two-photon Ca²⁺ imaging, the NPY⁺ neurons exhibited reliable locomotion-related activity, with subset displaying tight cross-correlation in the activity profiles (FIG. 8E, F).

The method for designing cortical lamina-specific promoter candidates is similar to the one used by the inventor to develop PaqR4. For example, to identify promoter regions that may confer a layer 4-specific expression pattern, SArKS was applied to an RNAseq dataset comparing transcriptomes of pooled cells found in successive sections of primate cortex (He 2017). In He, the cortex was divided into sections representing different cortical layer. Gene sets were then based on sequences recovered from each section and assigned to layers.

We performed principal components analysis (PCA) on the expression levels of layer-specific gene sets comparing cortical sections and identified 151 candidate motifs and 10 top-scoring primate L4 genes. Here the inventor did not consider chromatin accessibility because no ATACseq information accompanied the cortical dataset. A study has recently appeared online (Mich 2019) that includes primate ATACseq, but the data is not currently accessible. When it is accessible, the data will be incorporated into our promoter selection strategy, as for PV promoter search. We will also perform our own RNAseq and ATACseq analyses using primate virus-labeled neurons to supplement published datasets.

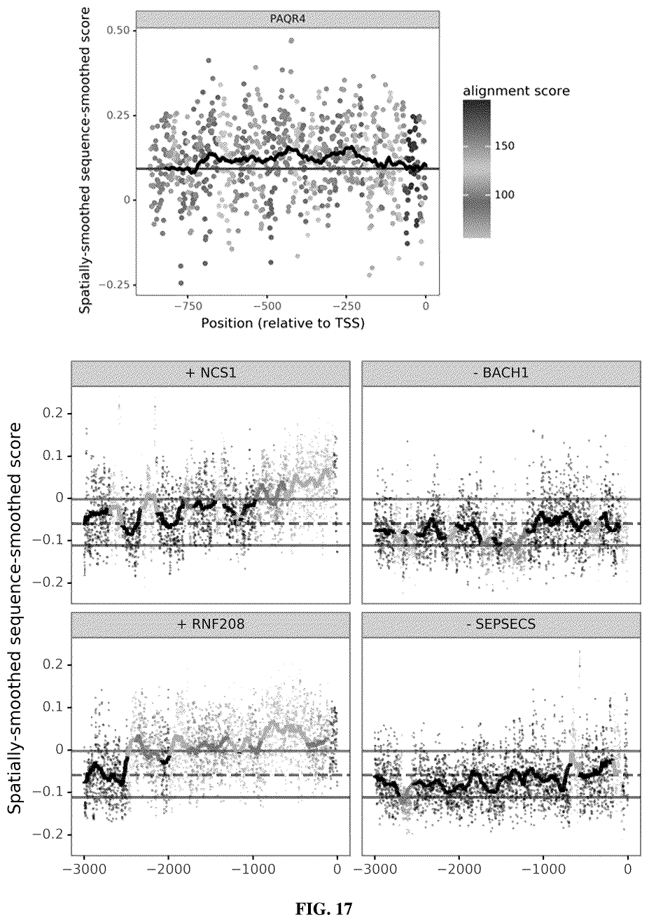

In mouse cortex (Allen Brain Atlas), 7 of 10 mouse gene orthologs showed layer-specific expression and 4 of 10 showed substantial enrichment in mouse cortical L4 over neighboring layers (including in area V1), a remarkable example of conserved spatial expression. In addition, some genes were expressed in excitatory neurons, while others were expressed in putative inhibitory neurons. We also identified genes and promoters that were preferentially excluded from L4. The L4 and non-L4 promoters included distinct sets of motifs and MMDs (FIG. 17). The promoter candidates have been incorporated into viral vectors for testing in mouse. Several vectors already show layer-specific expression in mouse V1 (FIG. 18). The process will be repeated for each cortical layer.

We tested whether or not h56D could restrict transgene expression to GABAergic neurons of another rodent. In the Mongolian gerbil, a popular model for auditory studies, forebrain GABAergic interneurons were also targeted with high specificity (HPC 98.4±1.6%, CTX 83.6±0.4%; FIG. 1F-G). In contrast, none of the promoters tested was active in the GABAergic neurons of rodent inferior colliculus (FIG. 9D), consistent with their mesencephalic origin and the corresponding lack of Dlx gene expression in the midbrain (Bulfone et al., 1993; Lahti et al., 2013). The effectiveness of h56D in mouse and gerbil forebrain suggests that it is broadly applicable in rodent models.

We also confirmed h56D efficacy in the marmoset cortex, where nearly all labeled neurons were GABAergic (specificity: 96.5±1.6%). Reporter expression was likewise detected across all cortical layers (coverage: 88.0±1.4; FIG. 1F-G). Robust and stable expression was also observed at eight sites in the visual cortex of four macaque monkeys: direct expression from the h56D promoter was seen at four of sites in two macaques, and expression restricted to putative GABAergic interneurons using two viruses was seen at three sites in two additional macaques (FIG. 10A-B).

To demonstrate that h56D viral vectors could be used to record functional responses from primate cortical interneurons, GCaMP6f was expressed in marmoset area MT (FIG. 2A) and rhesus macaque area V1 (FIG. 10D-E). Two-photon imaging of the marmoset cortex revealed differential visually-evoked fluorescence changes in response to distinct motion stimuli (FIG. 2B). Wide-field imaging at 3 injection sites in two macaques likewise uncovered robust fluorescence changes related to the repeated presentations of visual stimuli (FIG. 10E-F). These findings buttress our proposition that conserved gene-regulatory elements can engender cross-species cell type-specificity and can be used to reveal the functional characteristics of primate inhibitory neurons.

We demonstrate that single rAAVs can access forebrain GABAergic neurons broadly and that interdependent viruses can be employed to restrict access to specific excitatory and inhibitory subpopulations. Our success suggests that the general strategy of finding DNA sequences that are conserved between rodent and primate and of relying on combinatorial methods to refine genetic targeting is applicable to many neuron classes and will aid the transgenics-independent brain-wide interrogations of functionally significant cell populations.

Our set difference method for cell type-specific expression regulation represents a transgenics-independent way to target defined classes of neurons in the brain. While a fixed molar ratio of reporter and repressor vectors was used to enrich for NPY neurons, different promoters and ratios could access other cell subsets within and across traditional neuron classes for imaging and manipulation. Importantly, unlike recombinase-dependent techniques for expressing foreign proteins, the TetR-dependent approach is selective, tunable and reversible when regulated using injectable doxycycline or doxycycline added to animal chow (not shown). In addition, the TetR set difference technique can be used orthogonally with recombinases to target two cell classes, or jointly with recombinases, as demonstrated for SST⁺/NPY⁺ neurons above, to examine previously inaccessible neuronal circuit elements.

### Example 2—Methods

### Experimental Model and Subject Details:

All experiments were conducted in accordance with the National Institutes of Health guidelines and with the approval of the University of Texas at Austin and Columbia University Institutional Animal Care and Use Committees. Male and female C57BL/6J, 129S and Ai14 (Madisen et al., 2010) mice (8-16 weeks) were obtained from The Jackson Laboratory (Bar Harbor, ME) and bred in-house. NPY-Cre (Milstein et al., 2015) and PV-Cre (Scholl et al., 2015) were generated and bred in-house. PV-Cre; Ai14 mice were bred in-house. Mice were housed in groups of up to 4 animals and maintained on a 12 h reversed light/dark cycle. Surgeries and imaging experiments were conducted during the dark phase. Mongolian gerbils (3-5 weeks) were obtained from The Jackson Laboratory (Bar Harbor, ME) and bred in-house. Marmosets (1.5-4 years) and macaques (5-10 years) were housed at the Animal Resource Center of the University of Texas at Austin. Food and water were provided ad libitum, except as indicated below.

### AAV Assembly and Production:

To prepare the hybrid promoters, each human genomic enhancer domain was amplified by PCR from human genomic DNA and cloned in front of a cytomegalovirus (CMV) minimal promoter as a Not1-Nsi1 fragment; PCR primers containing these restriction enzyme sites were used to specify enhancer orientation within the construct. Enhancer sequence boundaries were as follows:

h12a-
(SEQ ID NO: 11)
GAAAGAGGTCCCCAGGACCA . . . CCAAGGCAAATTTTCACTGT
h12LR-
(SEQ ID NO: 12)
GCAAAATCTGTTTGGTCAAG . . . AAATTGCCAAACAACAGATA
h12R-
(SEQ ID NO: 13)
CAGCTGCAAACCCAAGAGGG . . . AAATTGCCAAACAACAGATA
h56D-
(SEQ ID NO: 14)
AGAAATAATGAAAATGAAAA . . . TTGCTGAATTATTCAAATTA
h56ii-
(SEQ ID NO: 15)
TCTGAGTCTCAGGGCAGAAG . . . AGCAAATCAGTGGTCTGAAG

To achieve TetR regulation, the CMV minimal promoter (GGGGGTAGG . . . GATCGCCTG (SEQ ID NO:16)) was interrupted by tandem palindromic TetO binding sites (TCCCTATCAGTGATAGAGA (SEQ ID NO:17)) (Hillen and Berens, 1994) separated by two base pairs (TC) starting 10 base pairs after the CMV TATA box (Yao et al., 1998). TetO sites were not present in vectors expressing TetR. The CMV minimal promoter was cloned as Nsi1-Sac1 fragment, such that all hybrid promoters were delimited by Not1-Sac1 sites. The somatostatin (SST) promoter (CCAGATCAA . . . GCAAGGAAG (SEQ ID NO: 18)) was amplified from mouse genomic DNA. The human PaqR4 promoter (GGAAGGGGA . . . GGAGAGACT (SEQ ID NO: 19)) was synthesized de novo (Integrated DNA Technologies). The 1.3 kb CaMKIIα promoter (AATTCATTA . . . GGCAGCGGG (SEQ ID NO: 20)) has been described previously (Dittgen et al., 2004). The CMV minimal promoter was not used with SST, PaqR4 or CaMKIIα promoters, which were all cloned as Not1-Sac1 fragments. Genes: EGFP, tdTomato, iCre (Shimshek et al., 2002), Flpo (Kranz et al., 2010; Raymond and Soriano, 2007) and TetR (Yao et al., 1998), each preceded by a Kozak sequence were cloned immediately behind a promoter as Sac1-Pst1 fragments. To impose recombinase dependence, the Kozak-gene cassette was inserted between asymmetric optimally-spaced loxP or frt recombination sites (Schlake and Bode, 1994; Seibler and Bode, 1997). In each viral construct, the promoter, gene, woodchuck post-transcriptional regulatory element (WPRE) and SV40 polyadenylation sequence were flanked by two inverted terminal repeats. Viruses were assembled using a modified helper-free system (Stratagene) as serotypes 2/1 or 2/7 (rep/cap genes). Serotype choice did not affect targeting specificity. Viruses were purified on sequential cesium gradients according to published methods (Grieger et al., 2006). Titers were measured using a payload-independent qPCR technique (Aurnhammer et al., 2012). Typical titers were >10¹⁰ genomes/microliter. For co-injections, the viruses were titer-matched and used in a 1:1 ratio (h12R-tdTomato:h12D-EGFP, SST-Cre:h56D-(EGFP)^{Cre}, SST-Flp:h56D-(EGFP)^{Flp}, PaqR4-Cre:h56D-(EGFP)^{Cre}), 1:2 ratio (h56D_{TetO4}-tdTomato:h12R-TetR and h56D_{TetO4}-GCaMP6f:h12R-TetR), 1:1:2 ratio (h56DTetO4-tdTomato:hSYN-(EGFP)^{Cre}:h12R-TetR and SST-Cre:h56D_{TetO4}-(tdTomato)^{Cre}:h12R-TetR), 1:2 ratio (hSYN-(EGFP_{FWD})^{Cre}:h56D-Cre and hSYN-(EGFP_{FWD})^{Cre}:CaMKIIα-Cre), and 1:1:1 ratio (h56D_{TetO4}-(EGFP)^{Cre}:h12R-TetR:SST-Cre).

### SArKS-Based Promoter Selection:

Suffix Array Kernel Smoothing (SArKS) finds motifs (k-mers) whose occurrence in a set of promoter sequences correlates with an input metric of differential expression. The general SArKS methodology is described elsewhere (Wylie et al., 2018). Here its specific application to PV⁺ interneuron targeting are covered. a mouse RNAseq data set was re-analyzed, where Cre mice were used to tag and isolate neocortical excitatory neurons, PV⁺ neurons and VIP⁺ neurons (Mo et al., 2015), using Kallisto (Bray et al., 2016) in order to better localize the most relevant transcription start sites (TSSs) for the expressed genes. Kallisto reported 73,912 distinct transcripts detected with nonzero estimated count in at least one of the analyzed samples. After filtering out transcripts that had low estimated counts or low average or low variance in transcripts-per-million (TPM) normalized expression levels, 29,164 distinct transcripts remained; these transcripts represented 11,857 distinct genes. To simplify downstream analyses, only a single transcript variant was retained having the highest average TPM for each gene. For each of the remaining transcripts, it was checked whether or not the TSS was located within a chromatin-accessible region in each of the neuron classes (as measured by ATACseq; Mo et al., 2015). In order to focus on those genes for which expression variability between neuron classes is most likely to be a function of promoter sequence as opposed to chromatin state, all genes were eliminated where the TSS was not contained within an accessible region in every neuron class. The upstream regions (˜3 kb) of the remaining 6,326 genes were examined using SArKS to uncover k-mers whose occurrence was correlated with a t-statistic comparing the TPM-normalized RNA transcript abundance in PV⁺ neurons versus PV⁻ neurons. SArKS first identified motifs by employing smoothing over subsequences by sequence similarity and then identified multi-motif domains (MMDs) by additionally smoothing over spatial proximity, using a permutation testing approach to establish statistical significance. The counts of how many times each uncovered motif occurred in a promoter region were then used as the feature vector for training a regression model to predict differential expression, again quantified as a t-statistic. The predicted scores from this regression model were then used to rank promoters by SArKS motif content, yielding 11 putative regulatory domains for experimental testing, one of which was for PaqR4.

### Cell Culture:

HEK293 cells were propagated according to standard methods. Briefly, cells were grown at 5% CO₂ in DMEM supplemented with 10% (v/v) FBS, 2 mM 1-glutamine and penicillin/streptomycin to 50-80% confluence (Gibco-BRL). Cell were transfected using jetPEI reagent (VWR) as recommended by the manufacturer. Indicated plasmid DNA mixes were incubated with transfection reagent in a 3:1 ratio. The cells were imaged 12-24 h post-transfection on an AXIOZoom V16 fluorescence microscope (Zeiss).

### Stereotaxic Surgery

### Mouse:

Both male and female mice were used for promoter characterization and slice electrophysiology studies. Only male mice were used for in vivo imaging studies. Mice were anesthetized with inhaled isoflurane (1-5% in oxygen), and body temperature was maintained at 37° C. Injections were performed using a stereotaxic apparatus (Kopf) fitted with a Nanoject II microinjector (Drummond Scientific). Pulled-glass pipettes back-filled with mineral oil were used to deposit virus mixes. For promoter characterization ˜20 nl virus was deposited bilaterally in hippocampal CA1 at depths 100 nm apart (from bregma: AP −2.2 mm; ML±1.5 mm; D −1.8 mm to −0.8 mm). For in vivo imaging studies, ˜30 nl virus was injected at six sites within the left CA1 region in three 10 nl pulses per site (from bregma: AP −2.2 mm; ML+1.5 mm; D 1.2, 1.1, 1.0 mm; and AP −2.5 mm; ML+1.6 mm, D 1.2, 1.1, 1.0 mm). Cortical injections were performed using a Micro4 controller (World Precision Instruments) to deposit ˜200 nl virus at the rate of 10 nl/min at a single location (from bregma: AP −2.2 mm; ML±1.5 mm; D −0.3 mm). Pipettes were left in place for 10 min following the injections. Animals were allowed to recover for at least 10 days post-injection.

### Gerbil:

Gerbils of both sexes underwent stereotaxic surgery for virus injection at 3-5 weeks of age. Gerbils were anesthetized with inhaled isoflurane (1-3% in oxygen), and body temperature was maintained at 37° C. Injections were performed using a stereotaxic apparatus (Kopf) fitted with a Nanoject II microinjector (Drummond Scientific). Pulled-glass pipettes back-filled with mineral oil were used to deposit virus mixes. In the inferior colliculus, 50 nL of virus was deposited bilaterally at depths 200 nm apart (from lambda: AP −1.25 mm; ML±1.15 mm; D −3.2 mm to −2.8 mm). In the hippocampus, 30 nL of virus was deposited bilaterally at depths 200 nm apart (from bregma: AP: −2.8 mm; ML: +1.8 mm; D: 1.6 mm to 0.2 mm). Cortical injections were performed using a Micro4 controller (World Precision Instruments) to deposit 200 nL of virus at the rate of 10 nL/min (from bregma: AP: −2.8 mm; ML: +1.8 mm; D: −0.3 mm). Pipettes were left in place for 10 min following the injections. Animals were allowed to recover for at least 10 days post-injection in group housing.

### Marmoset:

Adult marmosets were anaesthetized with isoflurane and placed in a stereotaxic frame. The body temperature was maintained at 36-37° C. and the heart rate, spO₂ and CO₂ were monitored throughout the procedure. The head was disinfected. and the surgery was performed under sterile conditions. A circular craniotomy of 4 mm diameter was performed on the cortex and the dura was removed. The virus was injected using Nanoject II (Drummond Scientific) with pulled and beveled glass pipettes with a tip diameter of 20-35 μm. The glass pipette was filled with mineral oil and front-loaded with the virus. The pipette was lowered into the visual cortex (D −0.5 mm), The virus was injected at 23 nl/sec up to a volume of 500 nl. The pipette was left in place for 5 min. Injection spread was assessed using trypan blue diluted 1:5 in virus mix. The craniotomy was closed using a custom-made chamber. The animals were then returned to their cages. Downstream procedures were conducted after a recovery period of 4-5 weeks.

### Macaque:

Surgical procedures, injection and expression screening were performed as described previously (Seidemann et al., 2016). After viral injection, widefield epifluorescence images of injection sites were taken weekly until the chamber was removed (see Seidemann et al., 2016). Red fluorescent protein (tdTomato) was imaged using 540 nm excitation and 565 nm dichroic filters. Green fluorescent protein (EGFP) was imaged using 470 nm excitation, 505 nm dichroic, and 520 nm emission filters.

### In Situ Hybridization:

Multiplexed in situ hybridization to indicated transcripts were performed using the RNAscope system (Advanced Cell Diagnostics). Whole brains from injected rodents were flash-frozen in OCT medium (Tissue Tek) using a dry ice/ethanol bath at 10-15 days post-injection. Cortical tissue from marmoset visual cortex was collected using a 4 mm biopsy punch (Integra) and immediately flash-frozen in OCT. All samples were cryosectioned at 12 μm (Leica CM3050S) and processed according to probe manufacturer instructions. Briefly, fixed and dehydrated sections were co-hybridized with proprietary probes (Advanced Cell Diagnostics) to neuronal marker transcripts, followed by differential fluorescence tagging. Signals in cells identified using DAPI staining were co-localized on an AXIOZoom V16 microscope (Zeiss).

### Immunostaining:

Immunohistochemistry was performed on 50 m sections of fixed mouse and marmoset brain and 25 μm sections of fresh frozen marmoset brain. Mice were sacrificed with an overdose of ketamine/xylazine, perfused with PBS, then 4% formaldehyde/PBS. Perfused brains were post-fixed overnight in 2% formaldehyde/PBS, then rinsed and stored in PBS until sectioned on a VT1000S vibratome (Leica). Marmoset brain was fixed for 48 hours in 4% formaldehyde/PBS, then rinsed and stored in PBS until sectioned. Fresh frozen marmoset tissue was sectioned on a CM3050S cryostat (Leica), mounted on Superfrost Plus glass slides (Fisher Scientific), and fixed using ice-cold acetone for 10 min. Free-floating mouse and marmoset sections were permeabilized with 0.5% Triton X-100/PBS and rinsed in PBS. All sections were blocked for 1 h in 5% Normal Goat Serum/0.3% Triton X-100/PBS, then incubated 48 h at 4° C. with indicated primary antibody diluted in blocking solution: rabbit anti-PV at 1:300 (Swant, PV-25/28), rabbit anti-NOS at 1:250 (Cayman Chemicals, 160870), rat anti-SST at 1:200 (Millipore, MAB354). The sections were washed three times with PBS and incubated with Alexa-conjugated secondary antibody (Invitrogen) at 1:500 in blocking solution. The sections were again washed in PBS and mounted on Superfrost Plus glass slides (Fisher Scientific) using DAPI Fluoromount-G (SouthernBiotech). Sections were examined on an AXIOZoom V16 fluorescence microscope (Zeiss); images were acquired on a TCS SP511 laser confocal microscope (Leica). Due to the thickness of the tissue, it was not always possible to accurately determine the number of cells in each field of view using DAPI staining. In addition, damage to marmoset tissue due to acetone fixation compromised DAPI staining.

Cell quantitation: Promoter specificity was examined using immunofluorescence and multiplexed in situ hybridization. Fluorescence analysis was performed during the initial examination of all viral vectors and was followed by in situ studies. A typical injection field covered up to 1 mm of brain tissue. For fluorescence analysis, 24, 50 μm tissue sections were collected per injection site per hemisphere. Cell were not counted in areas marked by needle penetration and concomitant tissue damage or areas where virus coverage was reduced, such as at extreme edges of injection sites. Counting was conducted manually, except as described below, on 20 μm maximum projections of confocal section z-stacks. DAPI staining was used to identify individual cells and to aid cell counting. For in situ studies, 60-80 12 μm sections were collected per hippocampal injection and 40-60 12 μm sections were collected per cortical injection. Non-consecutive sections were imaged to avoid double-counting cells that may have spanned neighboring sections. This also allowed for sampling a wider injection area. Z-stacks were not collected for in situ images. In the cortex, all cells were counted, and values are reported based on the total cell number. In the hippocampus, high cell densities precluded counting all cells and all fluorescent cells were counted instead. Occasionally, sectioning removed the nucleus of a labeled cell, eliminating the DAPI signal; if signal was unambiguous, the cell was counted. Most counts were performed manually. For determining weak versus strong reporter expression from the h12R and h56D promoters, images were analyzed using ImageJ. To determine and compare the distribution of reporter expression in GABAergic neurons from h12R and h56D, ImageJ was used to estimate mean fluorescent intensities. Briefly, in situ images with maximum coverage of the hippocampus (except the dentate gyrus) were selected for both h12R and h56D (mice selected were 10-11 days post-injection). For each image used, a threshold was selected manually to ensure maximum range of weak and strong spots. The particle analysis tool in ImageJ was used to determine the mean fluorescent intensity. Histograms of mean fluorescence intensities were made against the number of cells using a bin-width of 300 units, and cells exhibiting less than 2000 units were considered weakly expressing.

### In Vivo Imaging

### Rodents:

Mice were injected as described above. Following a 3-5-day recovery period, they were surgically implanted with a cylindrical imaging window—a 3 mm coverslip (Warner) glued (Norland, optical adhesive) onto a 3.0×1.5 mm steel cannula—and a steel head post to facilitate head-fixed imaging experiments. The surgical protocol was performed as previously described (Kaifosh et al., 2013; Lovett-Barron et al., 2014). Viral expression was assessed through the implanted window starting two weeks post-injection.

### Behavioral Training:

After recovery from surgery, mice were water-restricted (>85% pre-restriction weight was maintained) and habituated to head fixation under the two-photon microscope. Mice were trained to run on a fabric treadmill for water rewards. Following run training, animals were given a single session (˜1200 s) of discrete pseudorandom stimulus presentations while neural activity was monitored with two-photon calcium imaging. Ten stimuli each (tone: 200 ms, 5 kHz, 80 dB; blue LED: 100 ms; air-puff to snout: 100 ms) were delivered using a microcontroller system (Arduino) and custom written software, with a randomized inter-stimulus interval of 10-20 s. Mouse velocity was inferred from belt displacement digitized via and optical rotary encoder (Bourns Inc, ENS1J-B28-L00256L) attached to a microcontroller (Arduino).

### Two-Photon Imaging:

Imaging was performed using a two-photon microscope equipped with an 8 kHz resonant scanner (Bruker), controlled by Prairie View Software. The light source was a tunable femtosecond pulsed laser (Coherent) running at 920 nm. The objectives were either a Nikon 40×NIR or a Nikon 16x water-immersion (0.8 NA, 3.5 mm WD and 0.8 NA, 3.00 WD, respectively) in distilled water. Green fluorescence was detected with a GaAsP PMT (Hamamatsu Model 7422P-40); the signal was amplified with a custom dual stage preamp before digitization (Bruker). Images were acquired at 300 m×300 μm (512×512 pixels) field of view at 30 Hz (70-100 mW of power after the objective). Imaging data was motion corrected with a 2D Hidden Markov Model (Kaifosh et al., 2014). Segmentation was performed manually by drawing polygons around the somata of neurons expressing calcium reporter. Fluorescence signals were extracted as the average of all pixels within each polygon and relative fluorescence changed were calculated as describe in (Jia et al., 2011), with a uniform smoothing window t₁=10 s and baseline t₁=100 s.

### Marmosets:

Marmosets were injected with viral constructs as described above. The custom-made chamber included an insert with a coverglass at the bottom for optical access to the brain over the stereotaxic coordinates of area MT. In another sterile procedure a custom-made head post was also affixed to the skull using metabond (Parkell, New York) (Mitchell et al., 2015).

### Behavioral Training and Experimental Control:

After recovery from surgery, marmosets were food-restricted and habituated to head fixation under the two-photon microscope and trained to fixate visual targets (Mitchell et al., 2015). Experimental control was provided by the Maestro software suite, which collected eye movement data, controlled visual stimulation and provided juice reward (https://sites.google.com/a/srscicomp.com/maestro/).

Two-photon imaging: Viral expression was assessed by measuring fluorescence beginning 3 weeks after injection using a custom-made two-photon microscope equipped with resonant mirrors to allow for video rate sampling (Scholl et al., 2017). Fluorescence was detected using standard PMTs (R6357, Hamamatsu, Japan) and then amplified with a high-speed current amplifier (Femto DHPCA-100, Germany). Images were acquired at 400 μm×400 fields of view using a 16× objective (Nikon N16XLWD-PF, Japan). Imaging data were motion corrected using cross correlation (Guizar-Sicairos et al., 2008).

### Macaques Wide-Field Imaging:

Macaques were injected, and virally-encoded protein expression was assessed as described above. Recordings were performed at 3 sites in 2 animals. Signal could be detected 6-7 weeks post-injection, which was similar to the signal onset observed in direct CaMKIIα expression (Seidemann et al., 2016). Reliable signal has been recorded for up to 4 months post-expression. To date, imaging has been terminated only due to the deteriorating health of the chamber, rather than loss of reporter. These animals are still being used in related experiments. Therefore, no histological confirmation of cell type-specificity is yet available in macaques. To evoke a strong visual response in the primary visual cortex (V1), a large (6×6 deg²) sine wave grating was used at 100% contrast centered at (2.5-3.5) deg, which covered the retinotopic location of the infected area in V1 (0.5-1.0 deg). The stimulus had a spatial frequency of 2 cpd and orientation of 90 degrees. The mean luminance of the screen was set at 30 cd/m². The grating was flashed with a temporal frequency of 4 Hz, (100 ms on, 150 ms off) while the monkey was performing a fixation task. The behavioral task and widefield GCaMP data analysis in the macaque were performed as described previously (Seidemann et al., 2016),

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,202
U.S. Pat. No. 5,030,015
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Patent Publication No. 2005/0260186
U.S. Patent Publication No. 2006/0104968
U.S. Patent Publication No. 2004/0146987
PCT Application No. 95/06128
PCT Application No. 94/09699
EP1507865
Adesnik et al., Nature 490, 226-231, 2012.
Alexander et al., Proc Natl Acad Sci USA. 85(14):5092-6, 1988.
Allen et al., J. Neurosci. 31, 6542-6552, 2011.
Anderson et al., Science 278, 474-476, 1997.
Armstrong et al., Front Neural Circuits 6, 23, 2012.
Aurnhammer et al., Human Gene Therapy Methods 23, 18-28, 2012.
Baraban et al., Journal of Neuroscience 17, 8927-8936, 1997.
Beck et al., J. Bacteriol. 150, 633-642, 1982.
Bell et al., Nat Rev Genet.; 12(8):554, 2011.
Benson et al., Journal of Neuroscience 11, 1540-1564, 1991.
Benson et al., Neuroscience 46, 825-849, 1992.
Betley and Sternson, Hum. Gene Ther. 22, 669-677, 2011.
Borghuis et al., J. Neurosci. 31, 2855-2867, 2011.
Bourane et al., Science 350(6260), 550-554, 2015.
Bray et al., Nature Biotechnology, 34: 525-527, 2016.
Bulfone et al., Journal of Neuroscience 13, 3155-3172, 1993.
Caputi et al., Current Opinion in Neurobiology 23, 179-186, 2013.
Cembrowski and Menon, Trends in Neurosciences 41, 337-348, 2018.
Cembrowski et al., Neuron 89, 351-368, 2016.
Chin et al., Science 340, 759-762, 2013.
Christensen et al., Cell Reports 17(6), 1699-1710, 2016.
Cobos et al., Nat Neurosci 8, 1059-1068, 2005.
Cobos et al., Neuron 54, 873-888, 2007.
Davidson, Nature; 468(7326):911, 2010.
Dimidschstein et al., Nat Neurosci 19, 1743-1749, 2016.
Dittgen et al., Pnas 101, 18206-18211, 2004.
Ehrlich et al., Neuron 62(6), 757 771, 2009.
Elgar, Hum Mol Genet.; 5(Supplement_1):1437-1442, 1996/
Ellies et al., Genomics 45, 580-590, 1997.
Földy et al., Proc. Natl. Acad. Sci. U.S.A. 113, E5222-E5231, 2016.
Freund and Buzsáki, Hippocampus 6, 347-470, 1996.
Fuchs et al., Neuron 53, 591-604, 2007.
Fuentealba et al., Neuron 57, 917-929, 2008.
Gelman et al., J. Neurosci. 29, 9380-9389, 2009.
Ghanem et al., Genome Res. 13, 533-543, 2003.
Gossen et al., Science 268, 1766-1769, 1995.
Gossen, and Bujard, Pnas 89, 5547-5551, 1992.
Graybuck et al., Biorxiv.:525014, 2019
Grieger, J. C., Choi, V. W., and Samulski, R. J. (2006). Production and characterization of adeno-associated viral vectors. Nat Protoc 1, 1412-1428.
Guizar-Sicairos et al., Opt Lett 33, 156-158, 2008.
Gumucio et al., Mol Phylogenet Evol.; 5(1):18-32, 1996.
Gupta, A., Wang, Y., and Markram, H. (2000). Organizing principles for a diversity of GABAergic interneurons and synapses in the neocortex. Science 287, 273-278.
Haj-Dahmane, S., and Andrade, R. (1997). Calcium-activated cation nonselective current contributes to the fast afterdepolarization in rat prefrontal cortex neurons. J. Neurophysiol. 78, 1983-1989.
Han et al., Neuron 62, 191-198, 2009.
Hardison et al., Genome Res.; 7(10):959-966, 1997.
Harris et al., bioRxiv 143354, 2017.
He et al., Neuron 91, 1228-1243, 2016.
Hillen and Berens, Annu. Rev. Microbiol. 48, 345-369, 1994.
Hodge et al., Nature; 573(7772):61-68, 2019.
Hrvatin et al., Biorxiv.:570895, 2019.
Hu et al., Science 345, 1255263, 2018.
Huang et al., J. Neurosci. 34, 4976-4990, 2014.
Jia et al., Nat Protoc 6, 28-35, 2011.
Jinno and Kosaka, Neurosci. Res. 56, 229-245, 2006.
Jinno and Kosaka, Neuroscience 124, 797-808, 2004.
Kaifosh et al., Front Neuroinform 8, 80, 2014.
Kaifosh et al., Nat Neurosci 16, 1182-1184, 2013.
Kilduff et al., Trends in Neurosciences 34(1), 10-19, 2011.
Klausberger and Somogyi, Science 321, 53-57, 2008.
Kranz et al., J. Neurosci. 31, 14861-14870, 2010.
Kügler et al., Virology 311, 89-95, 2003.
Lahti et al., Acta Physiol (Oxf) 207, 616-627, 2013.
Lavdas et al., J. Neurosci. 19, 7881-7888, 1999.
Lee et al., J. Neurosci. 34, 11519-11525, 2014.
Lee et al., Nat Neurosci 16, 1662-1670, 2013.
Lein et al., Nature 445, 168-176, 2007.
Letovsky and Dynan, Nucleic Acids Res.; 17(7):2639-2653, 1989.
Li et al., Trends Genet.; 15(10):403-408, 1999.
Long et al., Cereb. Cortex 19 Suppl 1, i96-i106, 2009.
Lovett-Barron et al., Nat Neurosci 15, 423-30-S1-3, 2012.
Lovett-Barron et al., Science 343, 857-863, 2014.
Madisen et al., Nat Neurosci. 13(1): 133-140, 2010.
Marín and Rubenstein, Nat Rev Neurosci 2, 780-790, 2001.
Meyer et al., Pnas 108, 16807-16812, 2011.
Milstein et al., Neuron 87, 1274-1289, 2015.
Mitchell et al., J. Neurophysiol. 113, 3954-3960, 2015.
Miyoshi and Fishell, Cerebral Cortex 21, 845-852, 2011.
Mo et al., Neuron 86, 1369-1384, 2015.
Mo et al., Neuron; 86(6):1369 1384, 2016.
Muñoz et al., Science 355, 954-959, 2017.
Murray et al., Mamm. Genome 23, 587-599, 2012.
Murray et al., Nature Neuroscience 14(3), 297, 2011.
Nathanson et al., Front Neural Circuits 3, 19. 2009b.
Nathanson et al., Neuroscience 161, 441-450, 2009a.
Noè et al., Brain 131, 1506-1515, 2008.
Ovcharenko et al., Nucleic Acids Research 32, W280-W286, 2004.
Pan et al., Neuron, 2019.
Paul et al., Cell 171, 522-539.e20, 2017.
Petilla Interneuron Nomenclature Group et al., Nature Publishing Group 9, 557-568, 2008.
Pfeffer et al., Nature Publishing Group 16, 1068-1076, 2013.
Potter et al., Mol. Cell. Neurosci. 40, 167-186, 2009.
Quitsche et al., 1989. J Biol Chem. 264(16):9539-46, 1989.
Raymond and Soriano, PLoS ONE 2, e162, 2007.
Royer et al., Nat Neurosci 15, 769-775, 2012.
Rudy et al., Dev Neurobiol 71, 45-61, 2011.
Samulski et al., J. Virol. 63, 3822-3828, 1989.
Schlake and Bode, Biochemistry 33, 12746-12751, 1994.
Schoch et al., J. Biol. Chem. 271, 3317-3323, 1996.
Schoenenberger et al., Nat Commun 7, 11824, 2016.
Scholl et al., Neuron. 87(2): 424-436.2015.
Scholl et al., J. Neurophysiol. 117, 910-918, 2017.
Seibler and Bode, Biochemistry 36, 1740-1747, 1997.
Seidemann et al., Elife 5, 3771, 2016.
Shimshek et al., Genesis 32, 19-26, 2002.
Simon et al., J. Neurosci. 25, 6278-6285, 2005.
Soltesz and Losonczy, Nat Neurosci 21, 484-493, 2018.
Stuhmer et al., Cerebral Cortex 12, 75-85, 2002b.
Stuhmer et al., Development 129, 245-252, 2002a.
Sumiyama et al., Pnas 99, 780-785, 2002.
Tan et al, Nature Neuroscience 11(5), 538 540, 2008.
Taniguchi et al, Neuron 71, 995-1013, 2011.
Tasic et al., Nat Neurosci 19, 335-346, 2016.
Tenenbaum et al., J Gene Med 6 Suppl 1, S212-5222, 2004.
Thompson et al., Neuron 60, 1010-1021, 2008.
Tricoire and Vitalis., Front Neural Circuits 6, 82, 2012.
van den Pol et al., J. Neurosci. 29, 4622-4639, 2009.
Wang et al., Brain Research 1518, 9-25, 2013.
Watakabe et al., Neurosci. Res. 93, 144-157, 2015.
Welch, et al. EMBO J., 8(1):255-60, 1989.
Wichterle et al., Nat Neurosci 2, 461-466, 1999.
Woolfe et al., Plos Biol.; 3(1):e7, 2005.
Wylie et al., Bioinformatics, 35:20: 3944-3952, 2018.
Xu et al., Neuron 77, 155-167, 2013.
Yao et al., Hum. Gene Ther. 9, 1939-1950, 1998.
Zeisel et al., Science 347, 1138-1142, 2015.
Zerucha et al., J. Neurosci. 20, 709-721, 2000.

SEQUENCES

h56D (SEQ ID NO: 1)
AGAAATAATGAAAATGAAAAGTCCTTTCGTCATACAAGCAGGAAGCCCCATACTGCGAGAATTAATG
GCTACAGACCTGGGCATCCTTCAAATTATGAACCTTGAAACCACTGAGCCATTTATCAGAAGTCAATA
GAGATACTCAGAGTGCTCCATATAATGACAGCGACATACAGTCGTGCAAAAGGACAGTCACTATAATT
AGACACAAAGCAAAGACTACTTACCAATATACCCGTTCCTTTTTTTGTTGAGCAACTTCCACGCTCAGT
CAGTCTTCAGAATGGTTTAAAACCGATCAGTCTTGTCATTTTCTAGCATTCGCATTGTTACATTAGGAA
AAATGTTTTCTTTTCTTTTTTCCCCCTTCCTACTGTGAAACTTTGGGTTCGTAGCTCCCCAGGATCAATT
CTGAACAAAGCCTCCAGCTGCAGTGCCATCCAATTTGAAGCAGACATTGGGGACAATTTAAGGTTTTT
ATCCACAAGAAGGTTTTTTTCCATTCTCTTAAATGCAGCCATAATTAGAGTAATTTTTCATGTAGCCCG
CTGATTACAGCGTTTTTACCGTCAAAGATAATTACCTGTAATTTTCTTCCACTTTTAATACTAAAAAGC
CATCTTTATTTAGATTCAGGAACAGGAAAGGCGAAACAAAAGAGGGAAATTATTCTGTTATTCATACA
CAAATTGCAGAGACGTAGGACCTAAAATTGAAAATTAACCAAAATTATAATGCTGAAAAGAATGGAA
GAGGCTGCAGAAGTACAGCTGGTAGTGGGGCTTTGCTGAATTATTCAAATTAGTCGACATGCATGGGG
GTAGGCGTGTACGGTGGGAGGCCTATATAAGCAGAACTCTCCCTATCAGTGATAGAGATCTCCCTATC
AGTGATAGAGATCTCCCTATCAGTGATAGAGATCTCCCTATCAGTGATAGAGAGTTTAGTGAACCGTC
AGATCGCCTG
h56R (SEQ ID NO: 2)
GTCGACTAATTTGAATAATTCAGCAAAGCCCCACTACCAGCTGTACTTCTGCAGCCTCTTCCATTCTTT
TCAGCATTATAATTTTGGTTAATTTTCAATTTTAGGTCCTACGTCTCTGCAATTTGTGTATGAATAACAG
AATAATTTCCCTCTTTTGTTTCGCCTTTCCTGTTCCTGAATCTAAATAAAGATGGCTTTTTAGTATTAAA
AGTGGAAGAAAATTACAGGTAATTATCTTTGACGGTAAAAACGCTGTAATCAGCGGGCTACATGAAA
AATTACTCTAATTATGGCTGCATTTAAGAGAATGGAAAAAAACCTTCTTGTGGATAAAAACCTTAAAT
TGTCCCCAATGTCTGCTTCAAATTGGATGGCACTGCAGCTGGAGGCTTTGTTCAGAATTGATCCTGGGG
AGCTACGAACCCAAAGTTTCACAGTAGGAAGGGGGAAAAAAGAAAAGAAAACATTTTTCCTAATGTA
ACAATGCGAATGCTAGAAAATGACAAGACTGATCGGTTTTAAACCATTCTGAAGACTGACTGAGCGTG
GAAGTTGCTCAACAAAAAAAGGAACGGGTATATTGGTAAGTAGTCTTTGCTTTGTGTCTAATTATAGT
GACTGTCCTTTTGCACGACTGTATGTCGCTGTCATTATATGGAGCACTCTGAGTATCTCTATTGACTTCT
GATAAATGGCTCAGTGGTTTCAAGGTTCATAATTTGAAGGATGCCCAGGTCTGTAGCCATTAATTCTCG
CAGTATGGGGCTTCCTGCTTGTATGACGAAAGGACTTTTCATTTTCATTATTTCTGTCGAC
h12R (SEQ ID NO: 3)
CAGCTGCAAACCCAAGAGGGTCAGCATCATTTCACTGTATTCTCTTCTTGATTACAAGCCGGGCCCATC
AAACACAACATAATTACAGTAATTTCAGGTTTATTTATTCTAATGCAGTTTCCCCATCTCTCTGGTAATT
ATGAGCAATTTTTTCGCCCAGGGAATCTTTTTGCATTAACAAAAGAGATAACGCACTGAAAGCCAAAT
TTGCTGTGCATTGAGAAAAGGAAAAAAAAAAATCAAATAGGTGCGAGCTGCCATCTCTGCAATTCTCT
GGTACCGGAGCCGGCAAATTGCTTGCAGGTGTATGGAGCAAGCTTGTCAATGGCCAGGCCTCCAAATT
AGCAAATGCACAGCAGCAAAGTAATGAAGACAGACTTAGCAAAATTGCCAAACAACAGATACAATTG
CTCGAGATGCATGGGGGTAGGCGTGTACGGTGGGAGGCCTATATAAGCAGAACTCGTTTAGTGAACCG
TCAGATCGCCTG
mSST (SEQ ID NO: 4)
CCAGATCAAACCAGAGGGAGGTCAGGAAGGAGCTGAGATGTAAGATTAAAACCAGAAATGAAGCTAT
GCAATCGCGTTGGAGGGAGATGGTAGACAAGGATGGAGTGAGAAATACCTGACATAGAATGTTAGAG
GAGTATAAGAGAAAGAGAGATGATTAGAGGTAGAGAGAATGGGTAAGATTGGACAAGGGCAGGCAC
AGAGCTTCTGGGAAGGGAGGGAAGCAGAAAAGCAAGATGCCCACGCACTGCTTGTGTTTCTCCTTTGG
TTTTCATTTTTCTATTTTGTGTGTCTGCCAAGGGCCAGGTTCCCCAGTAGGAGAGATAGGAGGTCCCTT
GAAGATGCCACAGGACAGGTGACCGATGTGGCCTCTGAGACCCCAGCCTGAAGCCTGCAAAGATCTG
AGTATGCACAGGGACAAGACTTATTTTTCTGGGTTTGGGTTCCATAGCTCTAGATTCACCGCTCTTACT
GACTTTTCCAACACAGGAGCCAGTTTCAGTTTAATGCGTTGTTCTGTGCTCCCCCTGCCTGGAAGCTTT
GGCACTTCTTACCCTCTCTGCCTTGGCTTACAGATACAAAAGGAAATGTTATCTCAAGCTGTTTCAAGA
TACTGACTAACCCCTCTTACCTGTCCACTTCGTTTTTTAGGTGCAAGTGTTGAGAATGGTGTCAGAATG
CACCTAATTCATAGTGCGTGGTAGACATCAGCATTGTGCAAACCCTCTGCGTCACCTGACTTCTGAAGC
TGTGCATTTAAAAAAACCTCTACTGTTGGCTTCTCTGACTACCCTAAGATGTTAGTCCTTGAATTGCCT
GTGGAGATTACCTGTTCTATGCCAGGAATGGAGCTGGCATTTCCTTAATGAGCAGTTACTTTGAAAGC
GGCAGCCTTAGCTGGCAACCAGATGCAAACACTCAATTTGATGAACTAATAGACTTCCTTAAACTCCC
TCTGCACGTAGAAGAGAGGCCTAGACAGTCTGTTGATTCGTTACTCACATAGATATACAATGGCTACA
CACTGCCTTAATGTGTGCCCATCCTGGAGATTCCTAACCTAGCCGCTCAGGAATCTGTCAGAACACAC
ACAGCAGTGCACAAGAACCCTGGCCTTAGAGTCTAGATGCCTCTGCTCTTCCAACTGTACAGAGAAAT
GATCTGCCAGTCCCTTAAGCTTTCTTGGTTCAAGTTCTGAGGCTTGTTAGATGATTTCTGAGACTGATTC
CCAGGGCTGTTTCCAGGTGCCAAATGTAGGCTTTCTTTCTCCCCCTCCCTCCTGTGTGTGTGTGTGTGTG
TCTGTCTGTCTGTCTATCTGTCTCTCCAGTGGTTTTCTTTTGTTAAAATATAAAGATAGGCCGCTTGGAC
AAAGTGAGGTTCCTTTACAGCTCAATTTCATCCCTTTTTTCCTACAAGGCTTTAAGAGATGGAGGGAGA
GAATATAGTTCAGTCCTCTTAATTGCAAATTCATTCTGAGATTGTTTCCTAGACAGATCGCTCTAAGTC
TCACTCGCCATACAAAAAGTTAAAGGTGAATGCAAGTCCAGTAATCTGGGTACATTGACAGGTACCCA
ACTGAGTGTGATGATGTATTGCTAACCAAGGACTGAGTGATCTCTGTGTAATTAAGTGTGCTCCTATGT
GGCTGAAATATGGGAGCGGCATGTCAGCACTGAGTGAAGGTAAGATTGTTTGGTCTCTGTGGCATGGA
GAATTTCATGTGCCTGCGTGGGTGCAGGCTTTCTTTTTCTTTTTTTTAAAAAAATAAACCACTTTAGATC
GTGTCGCCTCCCCTCACTTCTGTGATTGATTTTGCGAGGCTAATGGTGCGTAAAAGCACTGGTGAGATC
TGGGGGCGCCTCCTTGGCTGACGTCAGAGAGAGAGTTTAAAAAGGGGAGACCGTGGAGAAGCTCCAT
AGCGGCTGAAGGAGACGCTACCGAAGCCGTCGCTGCTGCCTGAGGACCTGCGACTAGACTGACCCAC
CGCGCTCCAGCTTGGCTGCCTGAGGCAAGGAAG
hPaqR4 (SEQ ID NO: 5)
GGAAGGGGAGGAGGATCGTGGGCAGCGTTATGCTGAATAAGGGGTGCCTTCCCGGTCCCTGCTCAGG
GTGGACGGGGCGGAGGTCGACTTTGCTCCCCTGGCCTCCAATCTGTTTCCTGTCTATCCTCGGTAGGGC
CCCGCAAGGGTGCTCCTTGTGGGCGATAACTGGGAGCAAGTTGGGCCGGGCCCACGCTCCGAGAAGC
CTAGCGCGAAGGATAGGGCCTCTCCCGACGGCTGCGGGCGCGTGAGGCACGCCTTCAGAGGCCTGGG
TACCGTGGAGCGCCTTGCTGCACTCGGGAGTCCAGCCTGCGGAAAGATCACTTTGGAGCGGGGGCGTA
CCGGATGTAGGCCGGACCCGTCCGGCAGCACCTTGGACAGAGCCCCGTCTGCAGGGTAGGGCTAGGT
GGCAGGACTATGCCCCCGAGGGTGGGTGCCCAAAGGTACGGAGACCTGGGTGTCACGCGGAAAGCCC
GGATGCACAGTTCTGAGGGACGCGAGGTGCCAGGGTCACTCTAGCGCAGCCCGCAGGACCCAGAACG
TTGGGTCGCAAGCCCACAGCCACCCCATGCAAATGAGGCTGGGAGCGCGCACACTATGCTAGGAGGC
GAGGCCTGGGCGGCCTCGGGGCGGAGCCTCCCCGCCGGCCACGCCCATTGGCTCTCGCTGCGCCGACG
TCAGGAGCCCGGCGCGCGAAACGCTGGCCGGCCGGCGGGAACTAGGAGCCTGGGCGGAGCCTGGCGT
CCCCTCCCGCGTCCGGCCGCGCCCGTCCTCCTGGCTGCAGAGA
hPaqR4.P3 (SEQ ID NO: 6)
GCGGCCGCGAATTCGGAAGGGGAGGAGGATCGTGGGCAGCGTTATGCTGAATAAGGGGTGCCTTCCC
GGTCCCTGCTCAGGGTGGACGGGGCGGAGGTCGACTTTGCTCCCCTGGCCTCCAATCTGTTTCCTGTCT
ATCCTCGGTAGGGCCCCGCAAGGGTGCTCCTTGTGGGCGATAACTGGGAGCAAGTTGGGCCGGGCCCA
CGCTCCGAGAAGCCTAGCGCGAAGGATAGGGCCTCTCCCGACGGCTGCGGGCGCGTGAGGCACGCCT
TCAGAGGCCTGGGTTCCGTGGAGCGCCTTGCTGCACTCGGGAGTCCAGCCTGCGGAAAGATCACTTTG
GAGCGGTGTCACGCGGAAAGCCCGGATGCACAGTTCTGAGGGACGCGAGGTGCCAGGGTCACTCTAG
CGCAGCCCGCAGGACCCAGAACGTTGGGTCGCAAGCCCACAGCCACCCCATGCAAATGAGGCTGGGA
GCGCGCACACTATGCTAGGAGGCGAGGCCTGGGCGGCCTCGGGGCGGAGCCTCCCCGCCGGCCACGC
CCATTGGCTCTCGCTGCGCCGACGTCAGGAGCCCGGCGCGCGAAACGCTGGCCGGCCGGCGGGAACT
AGGAGCCTGGGCGGAGCCTGGCGTCCCCTCCCGCGTCCGGCCGCGCCCGTCCTCCTGGCTGCGGAGAG
ACTATGCAT
Rnf208.1 (SEQ ID NO: 7)
AGAGCGCGCCACGCTCCGCACGCACCTGCCGCCGTCGCCGCCGTGCCGAAACCCGCGCCCCGCGCCCC
ACGCCCGCGCCCGCGCCCCTCGGTGCCGCCCGGGCCCCGCCATCGCCTGAGGTCGCTGCGGTCGCCGC
CGGAGCCGCCGGAGCCCCCCGAGCTGCCGGGCCGAGGCGCGGGCGCCGCGTCCGGGCCTGCCTTTGA
GACAACCTCTGCGGCGGCGGCGCGCGGCCGGGACGCCAGGCTGGGGCAGGTGAGCGCAGGGGGCGG
GGGCCGGGGGCGGACTCAGCGCCCCTCCCCACGCGACGGGGGTCGGCCCTCGGGAAGGTGCGTCTGG
CCTTGGAGGCGGGGACCGGGGAGGGGGTTGGGTTCTGAGGCCCGCGGAGGTCGGGATCCCACGGCTG
AGGTCAGGGTCAGAGGTCGGGCGCCCGGTCCTTTGTGCAGGGCGAGGGCTAGGGTGGGGGTGGCCGG
GGCACTGCCCTCATGCCGTCTCTCACCTGCAGCTCAGGCCTGGCCCAGGCCTCGCCTCTGCTCCTGCCG
CGGAGCCTGCCGCCCCGGTCCTCCTGCAGCCAGCGCTTCGGCTAGCTGCCTTCCCTGGGCGCCCTGTCC
TGGAGCCATGGGGCCCACCCAGCCCCTGCCTGCCCCGATGTCCCGGCCCGCGGCCTGCTGACCTCGGC
TGCAGGGGGAGCCCCCCCCGCGCCCCCTCGCCAGCCTCAGCAGCCAGAGACCCTGGGTGAGACCCTGG
GCCAGACCCCCAGCCCAGGGCAGCCTCCCACCCCTCCCTGCCCCCGCCTGCCTCCCCCTCCCCCACCCC
TTCCTCCTCCTCCCAGGACTGGACCAGAGAAGCCACTGTGGCCACTGGGGGGGGCCCTGCACCCCCAA
CTCTCGCCGGCTGTCCCTAGGAGTCCACGGGCCGTCCGGGGCCCCCCCCAGGCCTGGCGGGACCAGGA
TGCTGCCCTG
Unc5d.1 (SEQ ID NO: 8)
GCGGCCGCTAGGAGCCATTCGAAATGAGGGAGCTTCTGAAAAGTTTGGGGATTCCGGAGTGTTCAGGA
CAACAGAATAGGGAATTCGAAAAAAAAAGAAAGCAAAAATAAAAATCGATTATACAAGCCTTAATGC
ACCCATGATTGTTATTCCCCACTTCTTTATTTATATGAATGAAGTTAGCATAGTTGCAGCAATGGAGTA
ACTCCATTATTATTTTTTCCCACTACGCATTTATTTTAAGTCTTTCTTTTTCTCAGGATGTTTTCCTGAGT
GATGTCTGATCTAATGGGAAAAAGTGGAAGTAGGAGAGGCAGGACTTTAGAAGGGCTCAAAGTAGAT
GAACAAAACGGAGGTCTCCTGGAAGCAGGTGACTGAGTTAGGCAACCGAGATGCCCACATATCTGTG
CGGCGTTCGCCCCAAGAGGCCAGCCGGCTCCGCTCAGCTCCCCGCGGCGTGGCCGCATCCCCGCCGCC
CGACTCCCAGCCCGCTTCCTCTCTCCGCGAGCGGGGGGGGGAGCTGCAGGCGGGGCCCGAAGCGCCCC
GGGGGCACGTGGAGCGGCCTCTGGCTCACCTGCGCCTCCCGGGCCCCAGCCCAGCGCCGCCGCGTCAC
CGCCCCTCCCGCGGCCCCGCCCCAGCCCGGCTCATTGGCTGCCGCTCGGAGGGGAGGGGAAGGGGCG
GCTGCGGGGGTCTCATTAGCGATGCGGACTCTCGCCTCCGCTCCGTAGTTCGGGGCCCGGCAGCGGCG
CGAGGGCTGGGAACTGCGCGGCGGGGACTGCGGGCGACTCCGGCATCCGCGCTGGCGGCAGCGGTCG
CCGCGCCGTGGGAAGCTATGGGGACGCGCCCTTTCTCGGGGTGCCCATTCAGCGGCGGCTCGGAGCTC
CCT
CMV Enhancer with NRSE (SEQ ID NO: 9)
CGGCCGCGAATTCTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTCCTACTTGGCAGTAC
ATCTACGTATTAGTCATCGCTATTACCATTCAGAACCACGGACAGCACCGAATTC
CB3 (SEQ ID NO: 10)
GAGGCATTTCCCCAACTGAAGCTCCTTTCTCTGTGATAACTCCAGCTGTGTCAAGTTGACACAAAACTA
GCCAGTACATGCCAGTAGGGCCTAAGTCTAAATACAGATGCAATAATTCAGTAACATGAAATGGGAA
AGAGTTTAATTGTGCTGGAGATTTTTTTTCTCCTCTTATGACACGTGGGATCGTGAACCTGTTCACCTG
AATGCTGTGTAATGAGGCCTAAGAGGCAAGTCATAAGAGTGCTTTGCAATGCTTTTGGAAAGTTTAAT
CTTATGGAACAATCACAGTATTCAAAGGGTGTGTACTTCTTCCAGAATCCTAGAAGGTGAGAGAGGTT
CCTGAATATTTGAAAAATAACACCCTCCCAATCTTGGTTAATGAACTGAAAAATTCGGCAGGCAAAAG
GCAGAAATAGTTCTTAAGTTACTTCAGAGCTGTAGAACTGAAACACACACTTGTTTAGTGTCCTTAAAC
TCAATTTAATTTTGAAGTGACACCAAAAATGTCTAAAAAGAGATGTTGATTTTAAATGAGAGTCTAAG
ACTAATAGTTATTTAAGAGAAAAATGTATTTTATGAAGTTACATTTCTTCTGCTAAAAAAAATTATCCA
GGAAAAAATATTACCTTAGAAAATCAAAATGAAAAAAAAAAGAAAATCAAAATGTAAATTGAAGAA
ATTGAAGACTATAACATTCAAAGATGTTGAGGATGCCATGTTTACAGCTTGTTAATCTGAGTTGATACA
AACTAAAAGAACTAATCTAGAAATGTTCTTTAAATTTTAAATTATATATGCTTTTGTACTAACAATAGT
ACCCCTATGACCAGGAAAAAATGTATATCTAAAATAAATATCTGAAGATATTTGTTGCAGCTGTTTGTC
ACAGTAGAACACTAAAAAAGATGGAATGTCTACAATTAGGATGGAAAACTATGTTATGTAAACATGG
GCTAATGGAGGAAGTGTCTACCGAGGAACAAACTTTTGTATGAGGATGTGACTTGGGTAGGCTGAGAT
TCAGTTTTCCCTGGAAATTAAGAGGTATATGAACAAAAGTAACTGACGTGTATTGGGTCAAATGCCCC
TTTACCTTTGTAAGCCACAGAAGGTAAGGTTAATACGTTTTATTGCACTACTTGCAGGACCTAGCAAAG
TACCTGACCCAAAAGCTGTTGATAAGTGTTCACTGAATGGATGAAAATATAAACAACTGGGCATGCAC
ATAATCTTTTTAAGTAAAAACAATATCCTTGTTATCCTCTCAGAGTTTGGCTTGCTTTGTGTATAATATC
AGTCTGAGATTTGTGTGAGAATCAGATAAGGTGATGAAAGTCAAAACTTACATCCATGAAAACTTTAG
AGTATCATGGCAGCAGGACTTGTCTGGAGAAGACAATTGGTTGCCTCACAATCCCACTATTGGAGGAA
ACACAAATGTGGTGAATTGCATATTCTATGACACACGTGAAACCTGTGGCGCTTCATCAGCTCGTTTTG
AAAGTTTATACCACACTGGTTTGCTTTTGTGTTACTGTAACCAGACACATAGGGTTCATTTTCAGCAGA
CCATAAGGTCTCAAAAGATGGACAATTAGATAACTTAGAAATACTTACGTAAAAGAAAACCTAAATA
CAGTATTTGTCATATTAAAATACCAATTGTAACATGTAGCCGGATATTTTTCCCACCTCTAATGATTTC
CAGTTTCTGGAAAAAAATCCCTCACCTAGAGATAGAAGCAGCGCAGCTGTAAAATCAGTGAGTGGGG
CTGCTACGGAGTCACTGGTTAGCCTGGTGACATTTCTTTCAGTTTCTACTTTGTAAGATGCAAGTAACT
AATGGCATCGATAAAATCACTTCCTTCCTAACATCTTAAATTCTTATAAGTTAATTCTACTACATTCCA
ATAATTCTGCTTCAAGCTCAAAAAGTAACAACAGCAAGAGCAGCAGACTCCTGCATTCTGCTGTCTCT
AAGCATAGCTCACATCTTAAACAGCCACGTGATGGTCTCCATTAGCGCAATATGAAGCATTGTTACAA
TTAACCACAGCAACGTATGCATTAATCAAATTAAACTAACACTTGACATCTGATTTTGTTCAAATACTC
AACTGCCTCGATAAATACTAAGTAGACAAAATCTCCACTGACGTGGTTTATCAGTCAGCTTTCCCTTCC
ATCTGAAAAAAAATCAAACAATTCTAGGTATGTTGCTTTACTCTAACATTCAGGAGTGAAAGCCTCCC
TGAACCTGGGGGATGTGAGGAGAAATGAGTCTGAGCAAGGGATCCCCACCACCTGCTGCTTCCTAGAC
TCCAAAACTCCAGCTCCAGCTATTTCCTGGGAAGAGAGAAATCGGAGGGGAGGGGAAGAAGGTTGGT
GAGAGCAAGAGGCGGGAGCTAGGAAAAGGAGGCAGGAGGAGGCGTGGCCCGGCCTGGGGCCGGCGG
GATAAATACAGAGAACTGGGTGCGGGGTGCGGAGAACTCCGGAGGACGCCCGAACGGAGCAGCACC
GCGGACAGCGCCCCGCCGCGCCGCGCCCAGCTCAGCCTGCGCAGCCCTCTCGCCCGAGGTTCGCGCTC
CGCGCACTCTCAAACTAGCCGCTGCACCACG
h12D (SEQ ID NO: 21)
GCGGCCGCGAATTCCTCGAGCAATTGTATCTGTTGTTTGGCAATTTTGCTAAGTCTGTCTTCATTACTTT
GCTGCTGTGCATTTGCTAATTTGGAGGCCTGGCCATTGACAAGCTTGCTCCATACACCTGCAAGCAATT
TGCCGGCTCCGGTACCAGAGAATTGCAGAGATGGCAGCTCGCACCTATTTGATTTTTTTTTTTCCTTTTC
TCAATGCACAGCAAATTTGGCTTTCAGTGCGTTATCTCTTTTGTTAATGCAAAAAGATTCCCTGGGCGA
AAAAATTGCTCATAATTACCAGAGAGATGGGGAAACTGCATTAGAATAAATAAACCTGAAATTACTGT
AATTATGTTGTGTTTGATGGGCCCGGCTTGTAATCAAGAAGAGAATACAGTGAAATGATGCTGACCCT
CTTGGGTTTGCAGCTGCTCGAGATGCAT

What is claimed is:

1. A method of inducing expression in a cell comprising contacting the cell with one or more nucleic acids encoding:
(i) a first promoter operably linked to a first expressible gene, and
(ii) a second promoter operably linked to a first recombinase, a transposase, or a repressor;
wherein the first promoter and the second promoter each induce expression in overlapping, but different, populations of neurons;
wherein expression of the recombinase or transposase by the second neuronal promoter can result in deletion or inversion of the first expressible gene, and wherein expression of the repressor can silence or prevent the expression of the first expressible gene; and
wherein the cell is preferably a neuronal cell.

2. The method of claim 1, wherein the first promoter and/or the second promoter are from a species that is different from the cell.

3. The method of claim 1, wherein the first promoter is a hybrid promoter comprising an enhancer and a minimal promoter.

4. The method of claim 3, wherein the first enhancer comprises or consists of h56D, h56R, h12R, h12D, mSST, hPaqR4, hPaqR4.P3, Rnf208.1, Unc5d.1, CB3, CMV enhancer with NRSE, or h12A.

5. The method of any one of claims 3-4, wherein the minimal promoter is a minimal CMV promoter, a minimal Na/K ATPase promoter, or a minimal Arc promoter.

6. The method of claim 1, wherein the second promoter is a hybrid promoter comprising an enhancer and a minimal promoter.

7. The method of claim 6, wherein the enhancer comprises or consists of h56D, h56R, h12R, h12D, mSST, hPaqR4, hPaqR4.P3, Rnf208.1, Unc5d.1, CB3, CMV enhancer with NRSE, or h12A.

8. The method of any one of claims 6-7, wherein the minimal promoter is a minimal CMV promoter, a minimal Na/K ATPase promoter, or a minimal Arc promoter.

9. The method of any one of claims 1-8, wherein the first promoter and/or the second promoter is a neuron-specific or neuronal promoter.

10. The method of claim 9, wherein the neuronal promoter is a pan-neuronal human synapsin promoter (hSYN), pan-neuronal mouse synapsin promoter (SYN), somatostatin (SST) promoter, CamKIIalpha, calbindin, CCK, or PaqR4.

11. The method of any one of claims 1-9, wherein the first promoter and/or the second promoter comprises a neuron-specific silencing element.

12. The method of any one of claims 1-11, wherein the expressible gene encodes an inhibitory nucleic acid sequence.

13. The method of claim 12, wherein the inhibitory nucleic acid sequence is a small interfering RNA (siRNA), a short hairpin RNA (shRNA) or micro RNA (miRNA).

14. The method of any one of claims 1-11, wherein the expressible gene encodes a reporter polypeptide, an ion channel polypeptide, a cytotoxic polypeptide, an enzyme, a cell reprogramming factor, a drug resistance marker, a drug sensitivity marker or a therapeutic polypeptide.

15. The method of claim 14, wherein the reporter polypeptide is a fluorescent or luminescent polypeptide.

16. The method of claim 14, wherein the expressible gene encodes GCaMP6f.

17. The method of claim 15, wherein the fluorescent or luminescent polypeptide is GFP, EGFP, or tdTomato.

18. The method of claim 14, wherein the cytotoxic polypeptide is gelonin, a granzyme, a caspase, Bax, Apo-1, AIF, TNF-alpha, a bacterial clostridium neurotoxin catalytic subunit, or a diphtheria toxin catalytic subunit.

19. The method of claim 14, wherein the reporter polypeptide comprises a destabilizing domain.

20. The method of any one of claims 1-19, wherein the recombinase is a Cre, Flp, or Dre recombinase.

21. The method of claim 20, wherein the recombinase comprises a destabilizing domain.

22. The method of claim 21, wherein the recombinase comprises an ER and/or PR domain.

23. The method of claim 21, wherein the recombinase comprises at least two destabilizing domains.

24. The method of any one of claims 1-23, wherein expression of the recombinase causes an inversion of or in the first expressible gene.

25. The method of claim 24, wherein the inversion results in a functional version of the first expressible gene.

26. The method of claim 24, wherein the inversion results in a non-functional version of the first expressible gene.

27. The method of any one of claims 1-26, wherein the second promoter results in expression of a first recombinase, and wherein the first recombinase is at least partially inverted or contains an inactivation region;
wherein the method further comprises contacting the neuronal cell with a third promoter operably linked to a second recombinase;
and wherein expression of the second recombinase can result in an inversion or deletion in the recombinase that activates enzymatic activity in the first recombinase.

28. The method of claim 27, wherein the third promoter is a hybrid promoter comprising an enhancer and a minimal promoter.

29. The method of claim 28, wherein the first enhancer comprises or consists of h56D, h56R, h12R, h12D, mSST, hPaqR4, hPaqR4.P3, Rnf208.1, Unc5d.1, CB3, CMV enhancer with NRSE, or h12A.

30. The method of any one of claims 28-29, wherein the minimal promoter is a minimal CMV promoter, a minimal Na/K ATPase promoter, or a minimal Arc promoter.

31. The method of any one of claims 27-30, wherein the third promoter is a neuron-specific or neuronal promoter.

32. The method of claim 9, wherein the neuronal promoter is PaqR4 promoter, a pan-neuronal human synapsin promoter (hSYN), somatostatin (SST) promoter, CamKIIalpha, or calbindin.

33. The method of any one of claims 27-32, wherein the first recombinase and the second recombinase are each independently a Cre, Flp, or Dre recombinase.

34. The method of any of claims 1-19, wherein the second promoter is operably linked to an operator, and wherein the repressor is TetR, MphR, VanR, TtgR or a ligand binding polypeptide fused to a kox-1 protein domain.

35. The method of any one of claims 1-34, wherein the one or more nucleic acids are comprised in a plasmid expression vector or an episomal expression vector.

36. The method of claim 35, wherein the vector is a viral expression vector.

37. The method of claim 36, wherein the viral expression vector is an adenovirus, adeno-associated virus, a retrograde virus, retrovirus, herpesvirus, lentivirus, poxvirus or papiloma virus expression vector.

38. The method of any one of claims 1-37, wherein the one or more nucleic acids are comprised in a single viral vector.

39. The method of any one of claims 1-37, wherein the one or more nucleic acids are comprised in at least two viral vectors.

40. The method of any one of claims 1-40, wherein the neuronal cell is comprised in a subject.

41. The method of claim 40, wherein the subject is a mammalian subject.

42. The method of claim 41, wherein the mammalian subject is a primate.

43. The method of claim 42, wherein the subject is a monkey or ape.

44. The method of claim 42, wherein the first expressible gene encodes a therapeutic gene product and wherein the subject is a human.

45. The method of claim 41, wherein the subject is a mouse.

46. The method of claim 45, wherein the mouse is a transgenic, knockout, or knock-in mouse.

47. An expression vector comprising h56D (SEQ ID NO: 1), h12R (SEQ ID NO: 3), h56R (SEQ ID NO: 2), h12D (SEQ ID NO: 21), mSST (SEQ ID NO: 4), hPaqR4 (SEQ ID NO: 5), hPaqR4.P3 (SEQ ID NO: 6), Rnf208.1(SEQ ID NO: 7), or Unc5d.1 (SEQ ID NO: 8).

48. The expression vector of claim 47, wherein the h56D, h12R, h56R, h12D, mSST, hPaqR4, hPaqR4.P3, Rnf208.1, or Unc5d.1 is operably linked to a promoter or an expressible nucleotide sequence.

49. The expression vector of claim 13, wherein the promoter is a minimal promoter.

50. The expression vector of claim 47, wherein the minimal promoter is a minimal CMV promoter, a minimal Na/K ATPase promoter, or a minimal Arc promoter.

51. The expression vector of any one of claims 47-50, wherein the promoter is operably linked to a first expressible gene.

52. The expression vector of claim 51, wherein the first expressible gene and/or the second expressible gene encodes an inhibitory nucleic acid sequence.

53. The expression vector of claim 52, wherein the inhibitory nucleic acid sequence is a small interfering RNA (siRNA), a short hairpin RNA (shRNA) or micro RNA (miRNA).

54. The expression vector of any one of claim 51, wherein the first expressible gene encodes a reporter polypeptide, an ion channel polypeptide, a cytotoxic polypeptide, an enzyme, a cell reprogramming factor, a drug resistance marker, a drug sensitivity marker or a therapeutic polypeptide.

55. The expression vector of claim 54, wherein the reporter polypeptide is a fluorescent or luminescent polypeptide.

56. A host cell comprising an expression vector in accordance with any one claims 47-54.

57. The host cell of claim 56, wherein the cell is a bacterial cell.

58. The host cell of claim 56, wherein the cell is a eukaryotic cell.

59. The host cell of claim 58, wherein the cell is a mammalian cell.

60. The host cell of claim 59, wherein the cell is neuron.

61. The host cell of claim 59, wherein the cell is a cancer cell.

62. The host cell of claim 56, wherein the expression vector is maintained episomally in the cell.

63. The host cell of claim 56, wherein the expression vector is integrated into the genome of the cell.

64. The host cell of claim 63, wherein a single copy of the expression vector is integrated into the genome of the cell.

65. A method of assessing the status of a cell comprising:
(a) expressing in the cell a vector in accordance with any one claims 47-54; and
(b) detecting the expression of the first expressible gene and/or the second first expressible gene, thereby assessing the status of the cell.

66. The method accordingly to claim 65, wherein one of said first expressible gene or said second expressible gene encodes a fluorescent or luminescent polypeptide and wherein detecting the expression comprises imagining the cell to detect expression of the fluorescent or luminescent polypeptide.


SEQUENCE LISTING (21 sequences)

SEQ ID NO: 1 - DNA, 966 bp, synthetic construct
  misc_feature 1..966: note = Synthetic oligonucleotide
  source 1..966: mol_type = other DNA
agaaataatgaaaatgaaaagtcctttcgtcatacaagcaggaagccccatactgcgagaattaatggctacagacctgggcatccttcaaattatgaaccttgaaaccactgagccatttatcagaagtcaatagagatactcagagtgctccatataatgacagcgacatacagtcgtgcaaaaggacagtcactataattagacacaaagcaaagactacttaccaatatacccgttcctttttttgttgagcaacttccacgctcagtcagtcttcagaatggtttaaaaccgatcagtcttgtcattttctagcattcgcattgttacattaggaaaaatgttttcttttcttttttcccccttcctactgtgaaactttgggttcgtagctccccaggatcaattctgaacaaagcctccagctgcagtgccatccaatttgaagcagacattggggacaatttaaggtttttatccacaagaaggtttttttccattctcttaaatgcagccataattagagtaatttttcatgtagcccgctgattacagcgtttttaccgtcaaagataattacctgtaattttcttccacttttaatactaaaaagccatctttatttagattcaggaacaggaaaggcgaaacaaaagagggaaattattctgttattcatacacaaattgcagagacgtaggacctaaaattgaaaattaaccaaaattataatgctgaaaagaatggaagaggctgcagaagtacagctggtagtggggctttgctgaattattcaaattagtcgacatgcatgggggtaggcgtgtacggtgggaggcctatataagcagaactctccctatcagtgatagagatctccctatcagtgatagagatctccctatcagtgatagagatctccctatcagtgatagagagtttagtgaaccgtcagatcgcctg

SEQ ID NO: 2 - DNA, 816 bp, synthetic construct
  misc_feature 1..816: note = Synthetic oligonucleotide
  source 1..816: mol_type = other DNA
gtcgactaatttgaataattcagcaaagccccactaccagctgtacttctgcagcctcttccattcttttcagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtaggaagggggaaaaaagaaaagaaaacatttttcctaatgtaacaatgcgaatgctagaaaatgacaagactgatcggttttaaaccattctgaagactgactgagcgtggaagttgctcaacaaaaaaaggaacgggtatattggtaagtagtctttgctttgtgtctaattatagtgactgtccttttgcacgactgtatgtcgctgtcattatatggagcactctgagtatctctattgacttctgataaatggctcagtggtttcaaggttcataatttgaaggatgcccaggtctgtagccattaattctcgcagtatggggcttcctgcttgtatgacgaaaggacttttcattttcattatttctgtcgac

SEQ ID NO: 3 - DNA, 490 bp, synthetic construct
  misc_feature 1..490: note = Synthetic oligonucleotide
  source 1..490: mol_type = other DNA
cagctgcaaacccaagagggtcagcatcatttcactgtattctcttcttgattacaagccgggcccatcaaacacaacataattacagtaatttcaggtttatttattctaatgcagtttccccatctctctggtaattatgagcaattttttcgcccagggaatctttttgcattaacaaaagagataacgcactgaaagccaaatttgctgtgcattgagaaaaggaaaaaaaaaaatcaaataggtgcgagctgccatctctgcaattctctggtaccggagccggcaaattgcttgcaggtgtatggagcaagcttgtcaatggccaggcctccaaattagcaaatgcacagcagcaaagtaatgaagacagacttagcaaaattgccaaacaacagatacaattgctcgagatgcatgggggtaggcgtgtacggtgggaggcctatataagcagaactcgtttagtgaaccgtcagatcgcctg

SEQ ID NO: 4 - DNA, 2017 bp, synthetic construct
  misc_feature 1..2017: note = Synthetic oligonucleotide
  source 1..2017: mol_type = other DNA
ccagatcaaaccagagggaggtcaggaaggagctgagatgtaagattaaaaccagaaatgaagctatgcaatcgcgttggagggagatggtagacaaggatggagtgagaaatacctgacatagaatgttagaggagtataagagaaagagagatgattagaggtagagagaatgggtaagattggacaagggcaggcacagagcttctgggaagggagggaagcagaaaagcaagatgcccacgcactgcttgtgtttctcctttggttttcatttttctattttgtgtgtctgccaagggccaggttccccagtaggagagataggaggtcccttgaagatgccacaggacaggtgaccgatgtggcctctgagaccccagcctgaagcctgcaaagatctgagtatgcacagggacaagacttatttttctgggtttgggttccatagctctagattcaccgctcttactgacttttccaacacaggagccagtttcagtttaatgcgttgttctgtgctccccctgcctggaagctttggcacttcttaccctctctgccttggcttacagatacaaaaggaaatgttatctcaagctgtttcaagatactgactaacccctcttacctgtccacttcgttttttaggtgcaagtgttgagaatggtgtcagaatgcacctaattcatagtgcgtggtagacatcagcattgtgcaaaccctctgcgtcacctgacttctgaagctgtgcatttaaaaaaacctctactgttggcttctctgactaccctaagatgttagtccttgaattgcctgtggagattacctgttctatgccaggaatggagctggcatttccttaatgagcagttactttgaaagcggcagccttagctggcaaccagatgcaaacactcaatttgatgaactaatagacttccttaaactccctctgcacgtagaagagaggcctagacagtctgttgattcgttactcacatagatatacaatggctacacactgccttaatgtgtgcccatcctggagattcctaacctagccgctcaggaatctgtcagaacacacacagcagtgcacaagaaccctggccttagagtctagatgcctctgctcttccaactgtacagagaaatgatctgccagtcccttaagctttcttggttcaagttctgaggcttgttagatgatttctgagactgattcccagggctgtttccaggtgccaaatgtaggctttctttctccccctccctcctgtgtgtgtgtgtgtgtgtctgtctgtctgtctatctgtctctccagtggttttcttttgttaaaatataaagataggccgcttggacaaagtgaggttcctttacagctcaatttcatcccttttttcctacaaggctttaagagatggagggagagaatatagttcagtcctcttaattgcaaattcattctgagattgtttcctagacagatcgctctaagtctcactcgccatacaaaaagttaaaggtgaatgcaagtccagtaatctgggtacattgacaggtacccaactgagtgtgatgatgtattgctaaccaaggactgagtgatctctgtgtaattaagtgtgctcctatgtggctgaaatatgggagcggcatgtcagcactgagtgaaggtaagattgtttggtctctgtggcatggagaatttcatgtgcctgcgtgggtgcaggctttctttttcttttttttaaaaaaataaaccactttagatcgtgtcgcctcccctcacttctgtgattgattttgcgaggctaatggtgcgtaaaagcactggtgagatctgggggcgcctccttggctgacgtcagagagagagtttaaaaaggggagaccgtggagaagctccatagcggctgaaggagacgctaccgaagccgtcgctgctgcctgaggacctgcgactagactgacccaccgcgctccagcttggctgcctgaggcaaggaag

SEQ ID NO: 5 - DNA, 784 bp, synthetic construct
  misc_feature 1..784: note = Synthetic oligonucleotide
  source 1..784: mol_type = other DNA
ggaaggggaggaggatcgtgggcagcgttatgctgaataaggggtgccttcccggtccctgctcagggtggacggggcggaggtcgactttgctcccctggcctccaatctgtttcctgtctatcctcggtagggccccgcaagggtgctccttgtgggcgataactgggagcaagttgggccgggcccacgctccgagaagcctagcgcgaaggatagggcctctcccgacggctgcgggcgcgtgaggcacgccttcagaggcctgggtaccgtggagcgccttgctgcactcgggagtccagcctgcggaaagatcactttggagcgggggcgtaccggatgtaggccggacccgtccggcagcaccttggacagagccccgtctgcagggtagggctaggtggcaggactatgcccccgagggtgggtgcccaaaggtacggagacctgggtgtcacgcggaaagcccggatgcacagttctgagggacgcgaggtgccagggtcactctagcgcagcccgcaggacccagaacgttgggtcgcaagcccacagccaccccatgcaaatgaggctgggagcgcgcacactatgctaggaggcgaggcctgggcggcctcggggcggagcctccccgccggccacgcccattggctctcgctgcgccgacgtcaggagcccggcgcgcgaaacgctggccggccggcgggaactaggagcctgggcggagcctggcgtcccctcccgcgtccggccgcgcccgtcctcctggctgcagaga

SEQ ID NO: 6 - DNA, 684 bp, synthetic construct
  misc_feature 1..684: note = Synthetic oligonucleotide
  source 1..684: mol_type = other DNA
gcggccgcgaattcggaaggggaggaggatcgtgggcagcgttatgctgaataaggggtgccttcccggtccctgctcagggtggacggggcggaggtcgactttgctcccctggcctccaatctgtttcctgtctatcctcggtagggccccgcaagggtgctccttgtgggcgataactgggagcaagttgggccgggcccacgctccgagaagcctagcgcgaaggatagggcctctcccgacggctgcgggcgcgtgaggcacgccttcagaggcctgggttccgtggagcgccttgctgcactcgggagtccagcctgcggaaagatcactttggagcggtgtcacgcggaaagcccggatgcacagttctgagggacgcgaggtgccagggtcactctagcgcagcccgcaggacccagaacgttgggtcgcaagcccacagccaccccatgcaaatgaggctgggagcgcgcacactatgctaggaggcgaggcctgggcggcctcggggcggagcctccccgccggccacgcccattggctctcgctgcgccgacgtcaggagcccggcgcgcgaaacgctggccggccggcgggaactaggagcctgggcggagcctggcgtcccctcccgcgtccggccgcgcccgtcctcctggctgcggagagactatgcat

SEQ ID NO: 7 - DNA, 959 bp, synthetic construct
  misc_feature 1..959: note = Synthetic oligonucleotide
  source 1..959: mol_type = other DNA
agagcgcgccacgctccgcacgcacctgccgccgtcgccgccgtgccgaaacccgcgccccgcgccccacgcccgcgcccgcgcccctcggtgccgcccgggccccgccatcgcctgaggtcgctgcggtcgccgccggagccgccggagccccccgagctgccgggccgaggcgcgggcgccgcgtccgggcctgcctttgagacaacctctgcggcggcggcgcgcggccgggacgccaggctggggcaggtgagcgcagggggcgggggccgggggcggactcagcgcccctccccacgcgacgggggtcggccctcgggaaggtgcgtctggccttggaggcggggaccggggagggggttgggttctgaggcccgcggaggtcgggatcccacggctgaggtcagggtcagaggtcgggcgcccggtcctttgtgcagggcgagggctagggtgggggtggccggggcactgccctcatgccgtctctcacctgcagctcaggcctggcccaggcctcgcctctgctcctgccgcggagcctgccgccccggtcctcctgcagccagcgcttcggctagctgccttccctgggcgccctgtcctggagccatggggcccacccagcccctgcctgccccgatgtcccggcccgcggcctgctgacctcggctgcagggggagccccccccgcgccccctcgccagcctcagcagccagagaccctgggtgagaccctgggccagacccccagcccagggcagcctcccacccctccctgcccccgcctgcctccccctcccccaccccttcctcctcctcccaggactggaccagagaagccactgtggccactggggggggccctgcacccccaactctcgccggctgtccctaggagtccacgggccgtccggggccccccccaggcctggcgggaccaggatgctgccctg

SEQ ID NO: 8 - DNA, 886 bp, synthetic construct
  misc_feature 1..886: note = Synthetic oligonucleotide
  source 1..886: mol_type = other DNA
gcggccgctaggagccattcgaaatgagggagcttctgaaaagtttggggattccggagtgttcaggacaacagaatagggaattcgaaaaaaaaagaaagcaaaaataaaaatcgattatacaagccttaatgcacccatgattgttattccccacttctttatttatatgaatgaagttagcatagttgcagcaatggagtaactccattattattttttcccactacgcatttattttaagtctttctttttctcaggatgttttcctgagtgatgtctgatctaatgggaaaaagtggaagtaggagaggcaggactttagaagggctcaaagtagatgaacaaaacggaggtctcctggaagcaggtgactgagttaggcaaccgagatgcccacatatctgtgcggcgttcgccccaagaggccagccggctccgctcagctccccgcggcgtggccgcatccccgccgcccgactcccagcccgcttcctctctccgcgagcgggggggggagctgcaggcggggcccgaagcgccccgggggcacgtggagcggcctctggctcacctgcgcctcccgggccccagcccagcgccgccgcgtcaccgcccctcccgcggccccgccccagcccggctcattggctgccgctcggaggggaggggaaggggcggctgcgggggtctcattagcgatgcggactctcgcctccgctccgtagttcggggcccggcagcggcgcgagggctgggaactgcgcggcggggactgcgggcgactccggcatccgcgctggcggcagcggtcgccgcgccgtgggaagctatggggacgcgccctttctcggggtgcccattcagcggcggctcggagctccct

SEQ ID NO: 9 - DNA, 398 bp, synthetic construct
  misc_feature 1..398: note = Synthetic oligonucleotide
  source 1..398: mol_type = other DNA
cggccgcgaattctattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattcagaaccacggacagcaccgaattc

SEQ ID NO: 10 - DNA, 2621 bp, synthetic construct
  misc_feature 1..2621: note = Synthetic oligonucleotide
  source 1..2621: mol_type = other DNA
gaggcatttccccaactgaagctcctttctctgtgataactccagctgtgtcaagttgacacaaaactagccagtacatgccagtagggcctaagtctaaatacagatgcaataattcagtaacatgaaatgggaaagagtttaattgtgctggagattttttttctcctcttatgacacgtgggatcgtgaacctgttcacctgaatgctgtgtaatgaggcctaagaggcaagtcataagagtgctttgcaatgcttttggaaagtttaatcttatggaacaatcacagtattcaaagggtgtgtacttcttccagaatcctagaaggtgagagaggttcctgaatatttgaaaaataacaccctcccaatcttggttaatgaactgaaaaattcggcaggcaaaaggcagaaatagttcttaagttacttcagagctgtagaactgaaacacacacttgtttagtgtccttaaactcaatttaattttgaagtgacaccaaaaatgtctaaaaagagatgttgattttaaatgagagtctaagactaatagttatttaagagaaaaatgtattttatgaagttacatttcttctgctaaaaaaaattatccaggaaaaaatattaccttagaaaatcaaaatgaaaaaaaaaagaaaatcaaaatgtaaattgaagaaattgaagactataacattcaaagatgttgaggatgccatgtttacagcttgttaatctgagttgatacaaactaaaagaactaatctagaaatgttctttaaattttaaattatatatgcttttgtactaacaatagtacccctatgaccaggaaaaaatgtatatctaaaataaatatctgaagatatttgttgcagctgtttgtcacagtagaacactaaaaaagatggaatgtctacaattaggatggaaaactatgttatgtaaacatgggctaatggaggaagtgtctaccgaggaacaaacttttgtatgaggatgtgacttgggtaggctgagattcagttttccctggaaattaagaggtatatgaacaaaagtaactgacgtgtattgggtcaaatgcccctttacctttgtaagccacagaaggtaaggttaatacgttttattgcactacttgcaggacctagcaaagtacctgacccaaaagctgttgataagtgttcactgaatggatgaaaatataaacaactgggcatgcacataatctttttaagtaaaaacaatatccttgttatcctctcagagtttggcttgctttgtgtataatatcagtctgagatttgtgtgagaatcagataaggtgatgaaagtcaaaacttacatccatgaaaactttagagtatcatggcagcaggacttgtctggagaagacaattggttgcctcacaatcccactattggaggaaacacaaatgtggtgaattgcatattctatgacacacgtgaaacctgtggcgcttcatcagctcgttttgaaagtttataccacactggtttgcttttgtgttactgtaaccagacacatagggttcattttcagcagaccataaggtctcaaaagatggacaattagataacttagaaatacttacgtaaaagaaaacctaaatacagtatttgtcatattaaaataccaattgtaacatgtagccggatatttttcccacctctaatgatttccagtttctggaaaaaaatccctcacctagagatagaagcagcgcagctgtaaaatcagtgagtggggctgctacggagtcactggttagcctggtgacatttctttcagtttctactttgtaagatgcaagtaactaatggcatcgataaaatcacttccttcctaacatcttaaattcttataagttaattctactacattccaataattctgcttcaagctcaaaaagtaacaacagcaagagcagcagactcctgcattctgctgtctctaagcatagctcacatcttaaacagccacgtgatggtctccattagcgcaatatgaagcattgttacaattaaccacagcaacgtatgcattaatcaaattaaactaacacttgacatctgattttgttcaaatactcaactgcctcgataaatactaagtagacaaaatctccactgacgtggtttatcagtcagctttcccttccatctgaaaaaaaatcaaacaattctaggtatgttgctttactctaacattcaggagtgaaagcctccctgaacctgggggatgtgaggagaaatgagtctgagcaagggatccccaccacctgctgcttcctagactccaaaactccagctccagctatttcctgggaagagagaaatcggaggggaggggaagaaggttggtgagagcaagaggcgggagctaggaaaaggaggcaggaggaggcgtggcccggcctggggccggcgggataaatacagagaactgggtgcggggtgcggagaactccggaggacgcccgaacggagcagcaccgcggacagcgccccgccgcgccgcgcccagctcagcctgcgcagccctctcgcccgaggttcgcgctccgcgcactctcaaactagccgctgcaccacg

SEQ ID NO: 11 - DNA, 40 bp, synthetic construct
  misc_feature 1..40: note = Synthetic primer
  source 1..40: mol_type = other DNA
gaaagaggtccccaggaccaccaaggcaaattttcactgt

SEQ ID NO: 12 - DNA, 40 bp, synthetic construct
  misc_feature 1..40: note = Synthetic primer
  source 1..40: mol_type = other DNA
gcaaaatctgtttggtcaagaaattgccaaacaacagata

SEQ ID NO: 13 - DNA, 40 bp, synthetic construct
  misc_feature 1..40: note = Synthetic primer
  source 1..40: mol_type = other DNA
cagctgcaaacccaagagggaaattgccaaacaacagata

SEQ ID NO: 14 - DNA, 40 bp, synthetic construct
  misc_feature 1..40: note = Synthetic primer
  source 1..40: mol_type = other DNA
agaaataatgaaaatgaaaattgctgaattattcaaatta

SEQ ID NO: 15 - DNA, 40 bp, synthetic construct
  misc_feature 1..40: note = Synthetic primer
  source 1..40: mol_type = other DNA
tctgagtctcagggcagaagagcaaatcagtggtctgaag

SEQ ID NO: 16 - DNA, 18 bp, synthetic construct
  misc_feature 1..18: note = Synthetic primer
  source 1..18: mol_type = other DNA
gggggtagggatcgcctg

SEQ ID NO: 17 - DNA, 19 bp, synthetic construct
  misc_feature 1..19: note = Synthetic primer
  source 1..19: mol_type = other DNA
tccctatcagtgatagaga

SEQ ID NO: 18 - DNA, 18 bp, synthetic construct
  misc_feature 1..18: note = Synthetic primer
  source 1..18: mol_type = other DNA
ccagatcaagcaaggaag

SEQ ID NO: 19 - DNA, 18 bp, synthetic construct
  misc_feature 1..18: note = Synthetic primer
  source 1..18: mol_type = other DNA
ggaaggggaggagagact

SEQ ID NO: 20 - DNA, 18 bp, synthetic construct
  misc_feature 1..18: note = Synthetic primer
  source 1..18: mol_type = other DNA
aattcattaggcagcggg

SEQ ID NO: 21 - DNA, 442 bp, synthetic construct
  misc_feature 1..442: note = Synthetic oligonucleotide
  source 1..442: mol_type = other DNA
gcggccgcgaattcctcgagcaattgtatctgttgtttggcaattttgctaagtctgtcttcattactttgctgctgtgcatttgctaatttggaggcctggccattgacaagcttgctccatacacctgcaagcaatttgccggctccggtaccagagaattgcagagatggcagctcgcacctatttgatttttttttttccttttctcaatgcacagcaaatttggctttcagtgcgttatctcttttgttaatgcaaaaagattccctgggcgaaaaaattgctcataattaccagagagatggggaaactgcattagaataaataaacctgaaattactgtaattatgttgtgtttgatgggcccggcttgtaatcaagaagagaatacagtgaaatgatgctgaccctcttgggtttgcagctgctcgagatgcat